(19) United States
(12) Patent Application Publication
(10) Pub. No.: US 2007/0122824 A1
(43) Pub. Date: May 31, 2007

(54) Method and Kit for Assessing a Patient's Genetic Information, Lifestyle and Environment Conditions, and Providing a Tailored Therapeutic Regime

(76) Inventors: Mark Rupert Tucker, Chadlington (GB); Robert Walton, Oxford (GB); Hubert Matthews, Oxford (GB); Anthony Miskin, West Yorkshire (GB)

Correspondence Address: GREENLEE WINNER AND SULLIVAN P C, 4875 PEARL EAST CIRCLE, SUITE 200, BOULDER, CO, 80301, US

(21) Appl. No.: 11/530,826

(22) Filed: Sep. 11, 2006

Related U.S. Application Data
(60) Provisional application No. 60/715,936, filed on Sep. 9, 2005.

(51) Int. Cl. C12Q 1/68; G06F 19/00; G06Q 50/00

(52) U.S. Cl. 435/6; 702/20; 705/2

(57) ABSTRACT

A method of determining a personalised therapeutic regime, comprising: receiving genetic information relating to a patient; determining genetic criteria relevant to a personalised therapeutic regime for the patient using the genetic information; receiving personal information relating to the patient; determining personal criteria relevant to the personalised therapeutic regime using the personal information; and combining the genetic criteria and the personal criteria to determine the personalised therapeutic regime for the patient.

This application claims the benefit of U.S. Provisional Application No. 60/715,936, filed Sep. 9, 2005, which is incorporated by reference to the extent there is no inconsistency with the present disclosure.

### BACKGROUND OF THE INVENTION

The present invention relates to a method for determining a personalised therapeutic regime.

Methods of genetic analysis are well known in the art. In particular “genotyping” or “haplotyping”, together with genomic and proteomic analysis have lead to the identification of certain genes, alleles, haplotypes, SNPs or other genetic indicia or loci that are connected or related in some way to certain diseases or conditions, or that may be suitable targets for drugs or gene therapy. Such loci may also be indicative of the validity or otherwise of particular therapies or Adverse Drug Reactions (ADRs) for that patient.

However, due to the inherent genetic differences between even closely related individuals, administration of certain drugs or therapies is not necessarily guaranteed success. This is because many genotypic or phenotypic factors (or environmental factors alone) can result in side effects or low adherence to the therapy. In particular, adherence to the dosage regime of prescribed drugs is often neglected by the patient, leading to a decrease in their therapeutic efficacy. This problem in particularly pronounced in large multi-cultural societies, where there is a high degree of genetic and cultural variance between individuals in a population.

Accordingly, there is a need to “personalise” treatment to particular individuals. This has proved to be difficult, given the time constraints that medical practitioners or therapists have available to them, particularly given a highly variant population.

Embodiments of the invention provide a system and method for providing personalised treatment or therapy to an individual.

### SUMMARY OF THE INVENTION

Surprisingly, the present inventors have discovered a system and method that obviates or ameliorates the above-described problems associated with conventional methods of therapy.

According to an aspect of the invention, there is provided a method of determining a personalised therapeutic regime, comprising obtaining quantitative and qualitative information relating to a patient, and using this information to obtain appropriate quantitative and qualitative criteria, and then combining the quantitative and qualitative criteria.

According to an aspect of the invention, there is provided a method of determining a personalised therapeutic regime, comprising: receiving at least one of genomic, proteomic, biochemical or metabolomic information relating to a patient; determining at least one of genomic, proteomic, biochemical or metabolomic criteria relevant to a personalised therapeutic regime for the patient using the or each genomic, proteomic, biochemical or metabolomic information; receiving personal information relating to the patient; determining personal criteria relevant to the personalised therapeutic regime using the personal information; combining the or each genomic, proteomic, biochemical or metabolomic criteria and the personal criteria to determine the personalised therapeutic regime for the patient.

In some embodiments, the determining of the personalised therapeutic regime for the patient comprises applying weightings to the or each genomic, proteomic, biochemical or metabolomic criteria and the personal criteria. Such weightings may be determined initially by a physician in accordance with standard medical practice, for instance. For example, it may be deemed that certain facts determined about a patient are more important for a diagnosis than others. For example, the fact that the patient has a history or family history of smoking or depression may be deemed more relevant than criteria such as height.

In some embodiments, the personalised therapeutic regime for the patient comprises applying weightings to the or each genomic, proteomic, biochemical or metabolomic criteria and the personal criteria within a structure reproducing that which an expert would use to make the decision.

Whilst the weightings and the mechanism by which they are combined can be established based on medical knowledge, they can also, or in addition, be dynamically updated from the results of analysis of outcomes from participants in the personalised medicine programme.

The basic structure of the therapeutic decision is intended to replicate that of an expert practitioner in that field. This is then updated as a result of analysis of the outcome of therapeutic manoeuvres in other participants in the personalised medicine programme.

In some embodiments, the personal information comprises information relating to the patient or information about the patient's lifestyle.

In some embodiments, the personal information comprises any one or combination of the following:
the ethnicity of the patient;
the age, weight, or Body Mass Index of the patient;
the sex of the patient;
the incidence of the condition in the patient's family (a so-called family history); and
the environmental conditions of the patient, such as the levels of stress in their home or workplace, the length of their working day, the physical or sedentary nature of their work, the amount of exercise that they take, their average alcohol consumption, their social and martial status; whether they suffer from sleep deprivation and so forth.

In some embodiments, where the method is directed to the cessation of smoking, it has been established that non-Caucasians with obesity are particularly susceptible to treatment using a nasal spray for administering nicotine. Thus, in some embodiments, where the patient is a non-Caucasian and has been diagnosed with obesity, this information is provided with great awaiting leading to an increased likelihood that the nasal spray, for instance, is prescribed.

In some embodiments, wherein the personal information is obtained in the form of a questionnaire. The questionnaire could be provided to the user over a communications network.

In some embodiments, the method is for predicting the likelihood of a non-smoker, such as a youth, becoming a smoker or becoming nicotine dependant.

In some embodiments, the or each genomic, proteomic, biochemical or metabolomic information is obtained from analysis of a sample from the patient.

The sample may be obtained by means of a self-use kit, similar to a blood-sugar level testing kit, which can be sent to the patient. The results can then be communicated and fed into the assessment system over a communication network.

In some embodiments, the kit may include a patient information leaflet. In some embodiments, the kit may include a plaster or other suitable adhesive. In some embodiments, the kit may include a sterilant, such as an antiseptic wipe. In some embodiments, the kit may include a dessicant. In some embodiments, the kit may include a lancet, which be an single-use lancet. In some embodiments, the kit may include a collection device, such as a device suitable for collecting tissue, protein, nucleic acid, or body fluids, such as blood, semen, saliva or cellular fluid, for instance. In some embodiments, the kit may include a blood spot collection card, sucha the SAFEspot™ card, described elsewhere.

Identification apparatus for establishing the genomic, proteomic, biochemical or metabolomic criteria are well known in the art. In some embodiments, this may include a cotton bud or swab. In some embodiments the apparatus includes a device similar for testing blood glucose levels, a so-called “finger-prick” device. In some embodiments, the apparatus is adapted to sample tissue and/or body fluids, especially blood, urine, semen or cellular fluid, or even mucus from a mucus membrane. In some embodiments, the apparatus tests the patients blood for the presence of genomic, proteomic, biochemical or metabolomic indicia.

The device may, in some embodiments, form part of a kit given, sold or sent to a patient.

As will be readily apparent, if the information is genomic, then the particular loci of the allele or SNP can be sequenced or analysed by RT-PCR, for instance, to determine the nucleotide identity of the allele or SNP. The sequences of the various alleles or SNP's are available on the internet, such as at the NCBI (National Center for Biotechnology Information) website, for instance. This is a mere matter of routine for the skilled person. If the information is physiological, biochemical or metabolomic, then suitable methods of identification, such HPLC, can be used to detect the molecule or molecules. Proteins or peptides can also be identified by such methods or by antibodies, again as is well established in the biological field.

As discussed elsewhere, certain genotypes are indicative of an adverse drug reaction to bupropion, in particular Zyban. Accordingly, if bupropion is contraindicated, then Nicotine Replacement Therapy (NRT) may be pursued as an alternative treatment. In some embodiments, NRT may also be contra indicated, or the dosage thereof may be determined, again by assessment or characterisation of at least one of the following: genomic, proteomic, biochemical or metabolomic information relating to the patient.

Incorrect dosage of nicotine, for instance in NRT, is not beneficial as levels that are too low may lead to smoking uptake whilst levels that are too high may tend to increase a patients nicotine dependence or cause nausea. However, as each individual will require separate levels, the present invention provides a method for determining the correct level, at a given point in time or for a given period, based on the assessment of the genomic, proteomic, biochemical or metabolomic information and personal criteria. For instance, heavy smokers may need higher levels of nicotine, at least initially. The strength of cigarettes smoked will affect this too, as a patient who smokes 20 strong cigarettes a day may need a different nicotine level to a patient smoking 25 weak or heavily filtered cigarettes a day.

As will be apparent from FIG. 26, various dosage levels can be used in NRT, and this will also depend on bupropion usage or contraindication. All this information is fed into and assessed by the system on a personal basis for each patient.

In some embodiments, the or each genomic, proteomic, biochemical or metabolomic analysis comprises any of or combination of the following:
genotyping;
haplotyping;
analysis of the patient's DNA;
analysis of the patient's RNA;
analysis of the patient's proteome; and
analysis of the patients metabolome, such as by examination of urine or other body fluids.

In some embodiments, relating to smoking cessation, the biochemical, metabolomic and/or proteomic information relates to the identification or characterisation of the levels of cotinine in a patient, for instance in patients blood. This may be monitored by a physician, in some embodiments. Cotinine is a metabolite of nicotine and is, therefore, an indication of nicotine levels. Should these levels need to be adjusted, then it will be readily apparent by assessing the levels of cotinine. For instance, in some embodiments, if the levels of nicotine are determined to be too low, these may be supplemented, so that the patient is less likely to take up smoking in order to enhance his nicotine levels. Levels of cotinine can be assessed by, for instance, a Nymox test strip, for example.

Similarly, the biochemical, metabolomic and/or proteomic information may relate to cholesterol levels. In some embodiments, the patient may be suffering for heart disease or is overweight. Methods of assessing cholesterol levels are well known in the art.

In some embodiments, the analysis may include or form a part of a biometric analysis or pharmacogenetic (Pgt) or pharmacogenomic (Pgx) analysis.

Pharmacogenomics can be thought of as the study of interindividual variation in whole genome or candidate gene SNP maps, haplotype markers, and alteration in gene expression or inactivation that may be correlated with pharmacological function or therapeutic response.

The analysis may be made substantially contemporaneously with the assessment, and preferably shortly before, so that results are as up to date as possible, it will be appreciated that in some circumstances, the analysis can have taken place some time before the assessment. This is particularly the case with genomic or haplotype analysis, which will vary through out a patient's life, unless altered by gene therapy.

In some embodiments, once the therapeutic regime for the patient has been determined, the personalised therapeutic regime is administered to the patient.

In some embodiments, once the personalised therapeutic regime has been administered to the patient, feedback information is received from the patient related to the effects of the personalised therapeutic regime.

In some embodiments, the method further comprises using the feedback information to determine an updated personalised therapeutic regime according to the effects of the personalised therapeutic regime on the patient.

In some embodiments the method further comprises using the feedback information in an aggregated form to make decisions about treatment of other patients within the personalised medicine programme.

In some embodiments, the method further comprises administering the updated personalised therapeutic regime to the patient.

In some embodiments, once the therapeutic regime has been administered, providing augmentation information to the patient in order augment the personalised therapeutic regime. In some embodiments, the augmentation information relates to any one or a combination of: cognitive behavioural therapy, managed care, and professional advice. These, especially the cognitive behavioural therapy, especially the Computerised Cognitive Behavioural Therapy (CCBT) may be provided over a communications network.

In some embodiments, the method comprises receiving results information relating to the results of the personalised therapeutic regime, and using said results information to determine a personalised therapeutic regime for a second patient. In particular, the personalised therapeutic regime outcome for a second patient with similar results, such as similar genotypic data and medical history, to the first, may then be similar

It is also envisaged that the medical history of a patient is fed into the system for assessment of the personalised therapeutic regime. This may be obtained from the patient's physician or inputted by the patient himself/herself, in which case an interface with the system may be appropriate.

In some embodiments, the comparing comprises comparing feature data obtained from the patient with corresponding reference feature data for the specified drug.

In some embodiments, there is provided a method of identifying a dosage of a specified drug, comprising:
receiving at least one of the following: genomic, proteomic, biochemical or metabolomic information relating to a patient;
determining at least one of genomic, proteomic, biochemical or metabolomic criteria; and
comparing the or each at least one of genomic, proteomic, biochemical or metabolomic criteria with reference criteria relating to an optimal dosage of said specified drug based on patients with said genomic, proteomic, biochemical or metabolomic criteria, to thereby determine the dosage of a specified drug.

In some embodiments, there is provided a method of determining a personalised therapeutic regime, comprising:
determining at least one of genomic, proteomic, biochemical or metabolomic criteria, relating to a patient;
determining personal criteria for the patient relating to personal information of the patient;
applying scoring criteria to the or each genomic, proteomic, biochemical or metabolomic criteria and the personal criteria to determine a scoring result for the patient;
determining the personalised therapeutic regime for the patient by comparing the scoring result against predetermined reference scoring information.

Embodiments the present invention are for the treatment or prophylaxis of smoking, in particular smoking cessation, the treatment or prophylaxis of depression and the treatment or prophylaxis of influenza.

In some embodiments, the method is directed to the treatment and/or prophylaxis of influenza. In some embodiments, this includes a prescription of appropriate drugs for treatment of flu. In some embodiments, the method includes identification of different flu types, for instance influenza A, influenza B and/or avian influenza, so-called “bird flu”. In particular, the method includes identification of whether or not the patient is in the correct therapeutic response window for particular prescribable drugs.

Two currently approved neuramidase inhibitor drugs for treatment of influenza, Relenza and TamiFlu, are known to work most effectively when taken within the therapeutic response window. By establishing pharmacogenetic and biochemical markers, for instance, correct dosage and drug prescription for treatment of influenza can be established.

In some embodiments, the method includes the use of a nasal swab in order to obtain cells and/or mucus from the nasal mucus membrane, thereby enabling characterisation of the viral type. A commonly-available nasal swab is a cotton bud, although more advanced versions are also known.

In one embodiment, the method relates to the treatment or prophylaxis of smoking or addiction thereto, in other words, assistance with the cessation of smoking. In some embodiments, the regime therapeutic regime comprises a regime to aid the patient cease smoking.

In some embodiments, the following genes and alleles may be assessed in a patient, such as a smoker or a previous smoker: the CYP2A6 gene; the CYP2D6 gene; the CYP2B6, the TPH allele; the DAT gene; the human dopamine beta-hydroxylase gene, DBH; the 5-HTT serotonin transporter; and the human monoamine oxidase A (MAO A) gene.

In some embodiments, the gene is CYP2B6. In some embodiments, the alleles to be assessed or determined are CC, CT, TT, and any of combinations thereof. Population kinetic analysis has established that bupropion total clearance via certain alleles was greater than others (Kircheiner et al, Pharmacogenetics, 2003, vol 13, No. 10, incorporated herein by reference).

Bupropion hydroxylation in vivo is thought to be catalysed by the gene product of CYP2B6 (Cytochrome P450 2B6). The characterisation of this gene, especially at the alleles mentioned, is included in some embodiments. CYP3A4 and CYP2E1 are also thought to be involved, albeit to lesser extent and are thus included in some embodiments.

Kircheiner et al described six distinct alleles, CYP2B6*2 to *7. These result from amino acid substitutions (R22C (64C>T, exon 1), Q172H (516G>T, exon 4), S259r (777C>A, exon 5), K262R (785A>G, exon 5), and R487C (1459C>T, exon 9). Six distinct alleles were defined based on the occurrence of the five genetic polymorphisms either alone or in combination. These SNPs may also be assessed in some embodiments.

Kircheiner et al showed that the CYP2B6*4 allele leads to higher clearance of bupropion compared to the wild-type, having clear therapeutic knock on effects, such as the requirement for increased or more frequent dosages of bupropion in order to retain effective plasma levels and therefore aid in maintaining smoking cessation or avoiding depression, or both.

Thus, in some embodiments, the present invention determines the presence of this alleles and prescribes higher or more frequent administration of bupropion to patient carrying said allele, especially if homozygous.

However, further alleles have since been characterised. We have established that the *4, *6 and *16 are particularly useful. Therefore the method according to some embodiments of the invention is directed to the characterisation of these CYP2B6*4, *6 and *16 alleles, either alone or in combination.

In some embodiments, the gene is DRD2. In some embodiments, the alleles to be assessed or determined are A1A1, A1A2, A2A2 and any of combinations thereof.

In some embodiments, the gene is TPH. In some embodiments, the alleles to be assessed or determined are AA, AT, and TATA and any of combinations thereof.

In some embodiments, the gene is DAT. In some embodiments, the alleles to be assessed or determined are any of the three allele combinations of the dopamine transporter gene (DAT). This is also known as SLC6A3.

SLC6A3 (Lerman et al, Health Psychology, 2003, Vol 22, No. 5) showed a significant DRD2×SL6A3 interaction effect on prolonged smoking abstinence and time to relapse. They had previously shown that persons with at least one copy of the 9-repat allele variant of SLC6A3 were significantly less likely to be smokers than those with no copies of the repeat allele (mostly the SLC6A3-10 genotypes). The association of SLC6A3 with smoking cessation history is significant. Furthermore, there is a higher prevalence of the rarer A1 or B1 alleles of DRD2 in smokers than non-smokers. In some embodiments, these alleles are characterised and positive identification thereof, or otherwise, is entered into the assessment system.

Lerman et al found that smokers with DRD2-A2 genotypes, those with SLC6A3-9 genotypes (i.e. combined DRD2-A2 and SLC6A3-9 genotypes), were had significantly higher abstinence rates at the end of treatment than DRD2-A2 and SLC6A3-10 genotypes. Thus, identification of these genotypes, in some embodiments, may lead to alternative therapeutic approaches. Patients with the DRD2-A2×SLC6A3-10 may need increased dosages of NRT or bupropion and over longer periods, for instance.

In some embodiments, the gene is the human dopamine beta-hydroxylase gene, DBH. In some embodiments, the alleles to be assessed or determined are the 444 G and/or A alleles, the 910 polymorphism, 1368 A and/or G alleles and any of combinations thereof.

In some embodiments, the method includes identification or characterisation of more than one allele or SNP. For instance, combinations of the DRD2 and SLC6A3 are useful. In some embodiments, the presence of DRD2-A2, in combination with SLC6A3-9, is indicative of a likelihood that the patient is a non-smoker, as it has been previously established that 62% of patients with genotype are non smoker, compared to 46% who are smokers. Thus, characterisation of this combination in a patient, is useful, particularly from a psychological point of view, if the patient is trying to cease smoking. Similarly, this combination is useful for determining whether or not a non-smoking patient is more or less likely to become a smoker. Characterisation of the combination of DRD2-A2 with SLC6A3-9 alleles mentioned above is also indicative of an increased ability to give up permanently, i.e. maintain smoking cessation.

Further examples include DRD2-A2 with SLC6A3-10. Characterisation of this allele in a patient indicates the greater need for “reinforcement,” such as further consultations and/or treatment in time, as patients showing this genotype are more likely to relapse, i.e. start smoking again following attempts to cease smoking.

Accordingly, there is provided a method of characterising at least one allele or SNP in order to determine a likely therapeutic outcome for the patient. In some embodiments, this includes characterisation of more than one allele and, in particular, combinations of alleles. In some embodiments, the alleles are DRD2-A2 and SLC6A3. In some embodiments the alleles are DRD2-A2 in combination with either SLC6A3-9 or SLC6A3-10.

It will also be appreciated that where reference is made to an SNP or an allele herein, that these terms are interchangeable, unless otherwise apparent. The terms refer to genetic polymorphisms but often result in protein isotypes having altered functionality. Thus, it will also be understood that reference to an SNP or an allele herein also includes reference to any resulting protein isotype.

Protein isotypes may be detectable by functional assays, particularly if the protein is an enzyme. They may also be detectable by antibodies or other recognition molecules, as any alteration in protein sequence may lead to a discernible conformational change, for instance.

In some embodiments, the gene is the human monoamine oxidase A (MAO A) gene. In some embodiments, the alleles to be assessed or determined are the 1460 C or T alleles. In some embodiments, 1460 C allele is assessed.

WO 01/38567 (Isis Innovation Limited), hereby incorporated by reference, teaches that the presence of at least one MAO A 1460 C allele indicates that the patient is less likely to be a heavy smoker than a patient who is homozygous for the MAO A 1460 T allele. Other alleles in close linkage with the 1460 alleles include the DBH 1368 A/G alleles, the presence of at least one MAO A 1368 A allele indicates that the patient is less likely to be a heavy smoker than a patient who is homozygous for the MAO A 1368 G allele. Further linked alleles are also disclosed, including MAO A uVNTR alleles 1 and 4, and MAO A allele 941 G. Suitable primers and methods for detecting these alleles are also disclosed.

The CYP2A6 cytochrome oxidase gene is involved in nicotine breakdown, as discussed below, and is included as a target for characterisation, in some embodiments.

The ANKK1 locus of the DRD2 (Dopamine D2 receptor) is also highly relevant to smoking treatment. In some embodiments, the characterisation of the T allele may be indicative that NRT is appropriate, whereas the presence of the C allele may indicative of bupropion proscription, if appropriate.

In some embodiments, the genomic, proteomic, biochemical or metabolomic information relates to at least one of the following: CD34, UGT1A1, and cytochrome oxidases. In some embodiments, the cytochrome oxidases are CYP2B6, CYP2D6, CYP2A6 and/or CYP2C19.

In some embodiments, the 14 genetic variants of the CYP2A6 gene are analysed, CYP2A6*1-*12 and the gene duplication *1×2, which can affect nicotine metabolism.

However, as discussed elsewhere, CYP2B6*4, *6 and/or *16 may be identified or characterised, in some embodiments.

In some embodiments, the genomic, proteomic, biochemical or metabolomic information relates to 5HT, DBH Monoamine Oxidase A (MAO) and/or DRD2. Heavier smokers tend to carry the DBH1368A allele, compared to light smokers, and are also lees likely to harbour the MAOA 1460C allele. Similarly, there is a higher prevalence of the DRD2 TAQIA variant allele, linked to reduced DRD2 receptor density in the brain, and are therefore, more likely to smoke more heavily than those without this allele. DRD2 TAQIA variant allele is found in the ANKK1 gene and is thought to affect substrate binding specificity.

Furthermore, females are more likely to suffer bupropion side effects if they carry the DRD2 TAQIA variant allele.

In some embodiments, the allele is the DRD2-141 Ins C allele. This may be important in for assessing bupropion proscription, as it may be more beneficial for those homozygous for this allele.

Conversely, those with the DRD2-141 Del C allele may benefit more from NRT. Thus, in some embodiments, the allele is DRD2-141 Del C.

The 5HT, DBH Monoamine Oxidase A (MAO) and/or DRD2 gene products are proteins which may be identifiable by the use of monoclonal antibody technologies. Alternatively, nucleic acids encoding these genes may be detected using suitable DNA or RNA microarays, for instance. These genes and their gene products are associated with the bioavailability of serotonin, however other genes and/or gene products associated with the serotonin and mood pathways are also envisaged.

The above-mentioned cytochrome oxidases are thought to be associated with nicotine breakdown. However, it will also be appreciated that other proteins, in particular enzymes, associated with breakdown of addictive drugs, such as nicotine or alcohol, are also envisaged.

In some embodiments, the identity of multiple alleles or SNPs is characterised.

Other suitable genes will be known to the skilled person and are as described in the accompanying references, all of which are hereby incorporated by reference.

A method of assessing the genetic information, i.e. detecting target nucleic acid sequences is described in GB 2338 301A (Zeneca Limited) hereby incorporated by reference.

The DRD2 genotype is associated with many drug-dependent conditions, including not only smoking, but also alcohol dependence (Lawford et 1995 Nature Medicine, 1, 337-341) where it was linked with response to bromocryptine treatment. Thus, in some embodiments, drug-dependence, including alcohol dependence and nicotine dependence, can be addressed by determining the DRD2 genotype of a patient.

Tryptophan hydroxylase (TPH) is the rate-limiting enzyme in serotonin biosynthesis and, as such, it plays a vital role in serotonin metabolism. It is thought to play a role in the reward and mood pathways and depression.

In some embodiments, the method may be used for the treatment or prophylaxis of heart disease, depression, including anxiety and/or panic, allergies, conditions associated with anticoagulant deficiencies such as haemophilia, dyspepsia, and/or elevated cholesterol levels.

In some embodiments, the method is for the treatment and/or prophylaxis of major depressive disorders, anxiety disorders of panic disorder, with and without agoraphobia, social phobia/social anxiety disorder, obsessive compulsive disorder, and post traumatic stress disorder.

In some embodiments, the identification and characterization of the above genes is important in the context of avoiding the Adverse Drug Reactions (ADRs). In particular, the pharmaceutical Zyban, comprising the active ingredient bupropion, is often prescribed to aid smoking cessation.

However, ADRs are seen in those patients carrying the DRD A1 allele. In fact, although this affects some men, 41% of women carry DRD A1 gene variant, so they are even more likely to suffer side effects from Zyban. In some embodiments, the present invention assesses the DRD A1/A2 allele and if the presence of the DRD A1 allele is detected, then this is fed into the system. In some embodiments, patients that have at least one copy of the A1 allele in their genome (i.e. those were that were A1 homozygous (A1-A1) or heterozygous (A1-A2)) are prescribed a treatment regime that does not include bupropion, including Zyban or Wellbrutin. As an alternative, these patients may be prescribed a course of NRT (Nicotine Replacement Therapy). Patients that are A2 homozygous (A2-A2) may be prescribed a course of bupropion, including Zyban or Wellbrutin.

Thus, in some embodiments, the patient is a female. It will be understood that when adverse drug-reactivity to Zyban is being assessed, that female patients will be particularly at risk and, therefore, the weighting given to females in this instance may be increased. This an example of how the various parameters entered into the system can have different weightings and lead to effective clinical and therapeutic outcomes. Further examples of ADRs that can, advantageously, be avoided by some embodiments of the present invention are discussed further below.

Over 100,000 Americans died in hospitals from ADR's to FDA-approved drugs, which were properly administered by licensed medical practitioners. Indeed, it is thought that approximately 7000 deaths occur in the US each year in hospitals due to ADR's and that medication errors occur in almost one in every five doses of prescribable drugs given in hospitals.

Thus, identification of patients that are susceptible to adverse drug reactions to particular drugs, is highly desirable. Accordingly, the present invention provides a method of identifying the likelihood of an adverse drug reaction in a patient to a specified drug, comprising using the methods taught in the present invention. In some embodiments, the method comprises receiving at least one of the following: genomic, proteomic, biochemical or metabolomic information relating to a patient; determining at least one of genomic, proteomic, biochemical or metabolomic criteria; and comparing these criteria with criteria linking certain drugs to adverse drug reactions based on patients with certain genomic, proteomic, biochemical or metabolomic criteria, to thereby determine whether a patient should be prescribed a particular drug or pharmaceutical product.

In some embodiments, the method includes determining that a patient has a particular genotype or displays a particular isotype of a protein and determining that at least one drug or pharmaceutical product is contra-indicated and, therefore, not suitable for administration to that patient.

In some embodiments, the method includes assessing more than one of each of the following: genomic, proteomic, biochemical or metabolomic information and/or combinations thereof. For instance, information from two genomic and one metabolomic or one proteomic and one biochemical indicia or markers may be assessed.

In some embodiments, the patient is a smoker. In some embodiments, genomic information relating to the patient is characterised. In some embodiments, this genomic information relates to at least one of the following: 5HT, DBH, and/or DRD2. In some embodiments, the genomic information to be characterised relates to cytochrome oxidases, especially CYP2D6, CYP2B6 and/or CYP2A6.

In some embodiments, the ANKK1 allele of DRD2 is characterised, particularly the presence or absence of the T or the C allele. In some embodiments the CYP2B6 allele is characterised, especially the *4, *6 and/or * 16 alleles thereof.

Further ADR's can also be assessed, such as those relating to a Iressa (Gefitinib), where the consequences of exposing non-responders to Iressa are significant. In some embodiments, polymorphisms in the TPMT gene are assessed, especially TPMT*2, *3A and/or *3C. In some embodiments, this leads to a individualised dosing of 6NP.

In some embodiments, the drug or pharmaceutical agent is Irinotecan (Campstar), which causes severe diarrhoea and neutropenia in 20-35% of patients and up to 5% fatality. Irinotecan is converted to SN-38 which is inactivated by UGT glucuronidation. UGT1A1*28 is a variant allele with reduced gene expression and glucuronidation. Homozygous UGT1A1 *28 (7/7 genotype) has 2-4 fold lower glucuronidation than wild-type) 6/6 genotype). Thus, characterisation of the Homozygous 7/7 genotype in UGT1A1*28 is indicative of 50% likelihood of neutropenia. Heterozygotes (6/7) show a 12.5% chance of neutropenia, whilst the wild type (Homozygous 6/6 genotype) show a roughly 0% chance of neutropenia.

Accordingly, in some embodiments, the present invention includes UGT genotyping, for example in cancer patients, to determine predisposed toxicity, i.e. an ADR.

In some embodiments, the drug or pharmaceutical agent is herceptin or trastuzumab, both of which also show ADRs for breast cancer patients. In some embodiments, therefore, the patient is suffering from breast cancer.

With respect to bupropion, other markers are also indicative of the success or otherwise of this well known active agent. Wellbrutin is a pharmaceutical often prescribed in relation to depression. In fact, Wellbrutin also comprises bupropion, except that it is at the twice the dose found in Zyban.

Where reference is made to Bupropion, it will be understood that this includes different dosages and products containing it, unless otherwise apparent. These may include Amfebutamone, as well as Zyban and Wellbrutin.

Therefore, in some embodiments, the present invention can also be applied to the treatment of depression. In some embodiments, this includes characterising certain SNPs or alleles and, on this basis, determining whether the patient should be prescribed Wellbrutin or not. As above, 41% of females should not be prescribed Wellbrutin, as this will lead to a greater risk of and ADR, especially as it is twice the dose of Zyban.

Thus, in some embodiments, the patient is being assessed for possible depression, is being treated for depression or has suffered an ADR to bupropion. In these instances, the present invention assesses the DRD A1/A2 allele and if the presence of the DRD A1 allele is detected, then this is fed into the system. In some embodiments, patients that have at least one copy of the A1 allele in their genome (i.e. those were that were A1 homozygous (A1-A1) or heterozygous (A1-A2)) are prescribed a treatment regime that does not include bupropion, in particular Wellbrutin. As an alternative, these patients may be prescribed a course of NRT (Nicotine Replacement Therapy). Patients that are A2 homozygous (A2-A2) may be prescribed a course of bupropion, including Wellbrutin.

With regard to depression, example conditions are Major Depression, Dysthymic Disorder, Unspecified Depression, Adjustment Disorder (with Depression) and Bipolar Depression.

This is advantageous for a number of reasons. Firstly, Wellbutrin is £180m market in the United States. However, there is some suggestion that it is being mis-prescribed due to a number of factors. One such factor is that the physician typically only has 7-11 minutes to make a correct diagnosis for depression, often due to work and time overload. It is widely thought that at least 50 mins is needed to correctly diagnose depression.

This is also some suggestion that physicians are under pressure form drug manufacturers to prescribe certain drugs, and together with extensive advertising, this may influence a physician's decision, especially when time is short.

As a result, it is thought that approximately 40% of current depressives receive medication, for instance Prozac, which has can lead to an enormous and perhaps unnecessary financial burden. In relation to ADRs, it simply may not be possible for the physician to determine whether the patients is in a risk group, with potentially dire consequences.

In some embodiments, therefore, the present invention provides a method of assessing or characterising the identity of certain genomic data, including SNPs and alleles and determining whether the patient is at risk from an ADR. In some embodiments, the ADR may be to bupropion, including Wellbrutin. However, it may also include Zyban, particularly given that 39% of depressives are also smokers.

Utilising the diagnostic intervention as a means of assessing the patients need for prescription drugs at an early stage enables patients who do not need medication to undergo Cognitive Behavioural Therapy, including Computerised Cognitive Behavioural Therapy (CCBT) in the first instance. This may be through a communications network, which may include the internet. In some embodiments, the CCBT may include that provided by VCC, available on the worldwide web, for instance at v-cc.com, DepTest.com, and g-nostics.com, branded under “DepTest”. Should this process fail, then the patient can be prescribed medication.

In some embodiments, the method includes the CCBT systems used by VCC, for instance. It will be appreciated that such systems do not include a biological assessment of the patient, such as an assessment of their genotypes, for instance, so they cannot ascertain the most appropriate form of medical treatment involving drugs without their own Pgt component. It is an advantage of the present invention that it encompasses both the CCBT and the biological assessment of the patient, as some patients may be treated correctly on the basis of CCBT alone, but others will require at least one medication at various dosages and time points, which can only be assessed by the present method.

Patient assessment by CCBT may, therefore, be achieved, in some embodiments, by use of the assessment software application available at VCC.com website. This is in relation to smoking cessation, but may also be easily adapted to be used for the assessment of depression. Some embodiments, the depression is of the panic or anxiety type. In some embodiments, the depression is as discussed elsewhere. In some embodiments, the method taught by Farvolden et al (J Meb Internet Res 2003; 5(3): e23) may be used. In some embodiments, this may be used in addition to or in replacement of the CCBT method available at the VCC.com website. The Farvolden et al paper is hereby incorporated by reference.

In some embodiments, the CCBT is conducted as a front line therapy unless it shows that medication is required. In some embodiments, the data from the CCBT is fed into the system, enabling further treatment optimisation, for instance in respect of medication.

It will be apparent that this method may not be suitable for schizophrenia or ‘high-end’ CNS drugs, as these patients may need medication of some sort, although ADRs can be avoided, in some embodiments.

However, for so-called ‘low-end’ users, which information may be entered into the assessment system of some embodiments of the invention, this approach is extremely attractive. For instance, there are 28 prescribable drugs in UK for depression. The majority of the drug market share is for these low-end users and represents about 40% of all patients, some of whom are unnecessarily being prescribed medication at huge direct and indirect cost to the national health services and about £1 B of lost productivity in UK alone.

Diagnostic intervention according to some embodiments of the present invention allows a reduction in medication costs and improved results whilst simultaneously keeping people at work as and when appropriate. It also allows the most appropriate medication to be prescribed downstream if deemed necessary. This process allows these patients to be identified and to receive improved diagnosis and a safer prognosis by being offered alternative medication. Further biomarkers are being identified all the time and can be added to the present system to increase its broad applicability, thereby helping to exclude patients from unsuitable medications.

In some embodiments, the present invention provides a method for assessing the likelihood of an ADR for a particular patient in respect of at least one drug. This can be fed back into the system, such that the drug is never mooted or is taken out of the likely appropriate therapies at an early stage.

In some embodiments, the drug is bupropion, including the pharmaceutical products Zyban or Wellbrutin, although it will be understood that other products which may comprise bupropion or its equivalents are also envisaged.

In some embodiments, analysis is undertaken of at least 2, 3 4, 5, 6, 7 8, 9 or 10 SNPs, especially at least 6. Data from this assessment, optionally together with biometric data, allows the system to recommend one treatment over another, thereby improving prognostic accuracy.

In some embodiments, the personal information or biometric information relates to one or more of the following:
the ethnicity of the patient;
the body mass index of the patient;
and as further discussed elsewhere.

In relation to the treatment for smoking, the personal information may additionally include:
the length of time the patient has been a smoker;
the average number of cigarettes smoked a day and its variation over time;
the type and/or strength of tobacco smoked; and
the type of filter used, if any, when smoking the tobacco.

In some embodiments, the personal therapeutic regime relates to suitable regime for treating or ameliorating any one or combination of the following: obesity, autoimmune diseases such as hayfever and arthritis, heart disease, AIDS, cancer, including breast cancer, prostate cancer, bowel cancer, testicular cancer, cancers of the blood, and lung cancer.

Embodiments are the smoking cessation, depression treatment and influenza treatments discussed above.

In some embodiments, any treatment that requires pharmaceutical or non-pharmaceutical medication and ‘management’ over the course of treatment is envisaged in relation to improving the curative treatment process.

In some embodiments, any treatment recommendation that arises from identification of an individual's predisposition to a genetic condition or probable treatment requirement that requires or incorporates lifestyle modification and/or vaccination or other such preventative strategies is also envisaged.

In another aspect of the invention, there is provided a method for determining a personalised therapeutic regime, comprising: a memory adapted to store at least one of genomic, proteomic, biochemical or metabolomic information relating to a patient; a memory adapted to store personal information relating to the patient; a determination processor adapted to determine at least one of genomic, proteomic, biochemical or metabolomic criteria relevant to a personalised therapeutic regime for the patient using the or each genomic, proteomic, biochemical or metabolomic information, to determine personal criteria relevant to the personalised therapeutic regime using the personal information, and to combine the or each genomic, proteomic, biochemical or metabolomic criteria and the personal criteria to determine the personalised therapeutic regime for the patient.

In another aspect of the invention, there is provided a system for determining a personalised therapeutic regime, comprising: a terminal adapted to receive a user indication of personal information relating to patient; a data store adapted to store said personal information relating to the patient; a testing sub-system adapted to perform at least one of genomic, proteomic, biochemical or metabolomic testing on a sample from the patient to obtain at least one of genomic, proteomic, biochemical or metabolomic information relating to the patient; a data store adapted to store the or each genomic, proteomic, biochemical or metabolomic information; a determination sub-system adapted to determine the or each genomic, proteomic, biochemical or metabolomic criteria relevant to a personalised therapeutic regime for the patient using the or each genomic, proteomic, biochemical or metabolomic information, to determine personal criteria relevant to the personalised therapeutic regime using the personal information, and to combine the or each genomic, proteomic, biochemical or metabolomic criteria and the personal criteria to determine the personalised therapeutic regime for the patient.

In another aspect of the invention, there is provided a server for use in a system for determining a personalised therapeutic regime, comprising: a data store adapted to store personal information relating to a patient; a data store adapted to store at least one of genomic, proteomic, biochemical or metabolomic information relating to the patient; and a determination processor adapted to determine at least one of genomic, proteomic, biochemical or metabolomic criteria relevant to a personalised therapeutic regime for the patient using the or each genomic, proteomic, biochemical or metabolomic information, to determine personal criteria relevant to the personalised therapeutic regime using the personal information, and to combine the or each genomic, proteomic, biochemical or metabolomic criteria and the personal criteria to determine the personalised therapeutic regime for the patient.

Embodiments of the invention therefore take bioinformatics and create a relevant output in such a way that it is user-friendly to both patient and clinician.

Personalised treatment (medicine) relates to identifying adverse reactions to existing drugs such that those drugs can be avoided in specific groups of people (Curative) when treating a disease or medical condition—such as within our approach, identifying drugs that will be particularly effective for treatment of an illness in an individual, identifying someone's predisposition to a specific disease such that lifestyle adjustment can be made in advance to delay or avoid the onset of the disease (preventative). A great deal of research investment is being made here as this is a likely driver of pharmaceutical sales downstream. It is hugely expensive though and ethically controversial.

Personalised treatment also relates to the design and delivery of new drugs that are tailored to specific groups of individuals that do not react adversely (Curative). This is an area almost entirely controlled by major pharmaceutical companies drawing on enormous resources. It is though that the end product is such that in 6-8 years time or so all new drugs will only be approved by the FDA etc if they are associated with a diagnostic to identify people who may react adversely.

Thus, identifying those patients that may suffer from an ADR (Adverse Drug Reaction) is highly important commercially as well as being highly desirable from a therapeutic efficacy point of view.

It will be apparent that as more SNPs and other criteria are determined which are linked to therapeutic or ADR outcomes for particular patients, the present invention can be applied thereto.

The benefits of this are that a virtual network of contractors could be used without fixed overheads, in that two tests belonging to say Company X and Y could use the system according to the present invention to exchange data between separate and different laboratories while the same (or different) patients would not know the difference. The user interface could be consistent whilst the actual product may be analysed by separate labs and the results sent back to the same source.

### BRIEF DESCRIPTION OF THE FIGURES

Embodiments of the invention will now be described, by way of example, and with reference to the accompanying drawings in which:

FIG. 1 shows a DxS SAFESpot™ device (a blood collection card) suitable for use with embodiments of the present invention. Four sample spots are shown where the blood is adsorbed onto the proprietary matrix;

FIG. 2 is a graph of standard DNA and SAFEspot DNA;

FIG. 3 shows genotyping using DNA from SAFEspot;

FIG. 4 is a schematic of the Scorpions system, a homogeneous or closed-tube platform for PCR analysis;

FIG. 5 is a graph showing reaction profile data generated using either 3000, 300 or 30 copies of genomic data using the Scorpions reaction;

FIG. 6A is a graph showing data generated using dilutions of genomic DNA ranging from 16,000 to 1.6 copies per Scorpions reaction;

FIG. 6B shows the data of FIG. 6B re-plotted on a logarithmic scale;

FIG. 7 is a graph showing the detection of an RNA transcript. The second lower trace was obtained using genomic DNA and shows that the Scorpions primer is only detecting PCR products derived from RNA;

FIG. 8 shows three traces from three separate Scorpions specific to three different RNA transcripts. Three separate dyes were used to allow differential detection of the three species;

FIG. 9 is a graph showing data generated using a Cepheid SmartCycler instrument. Scorpions primers were used to rapidly detect low levels of Bacillus spp;

FIG. 10 shows the data of FIG. 9 re-plotted;

FIG. 11 is a plot showing end point genotyping of the MTHFR gene using allele specific primer Scorpions. The ratio of the two fluorescent dyes indicates the genotype;

FIG. 12 is a plot showing end point genotyping of the NAT2 gene using allele specific probe Scorpions;

FIG. 13 is a plot showing real time single tube genotyping of the a BRCA1 polymorphism using allele specific primers;

FIG. 14 shows a comparison of Scorpions to both Molecular Beacons and Taqman detection systems, relating to the MTHFR gene;

FIG. 15 shows a comparison of Scorpions to both Molecular Beacons and Taqman detection systems, relating to the CYP gene;

FIG. 16 shows a comparison of Scorpions to both Molecular Beacons and Taqman detection systems, relating to the BRCA1 gene;

FIG. 17 is a schematic diagram of a system according to a general embodiment of the present invention;

FIG. 18 is a schematic diagram of a system according to an embodiment of the present invention;

FIG. 19 is a flow chart of a method according to an embodiment of the present invention;

FIG. 20 shows an embodiment of the invention that enables feeding back of information relating to the effectiveness of the personalised therapeutic regime, whether during or at the end of the personalised therapeutic regime. This has the particular benefit that it enables personalised therapeutic regime for patients to be constantly tested and evaluated. This enables the information relating to the relevance of genetic criteria and/or personal criteria stored in the decision matrix 60 to grow both in size and accuracy over time.

FIG. 21 shows a specific flowchart relating to the NicoTest™. This combines the personal therapeutic regime discussed above with a number of interactive, moderated on line support program features. There are two main components to the NicoTest™ method;

FIG. 22 shows the two main components to the NicoTest™ solution, diagnosis and the online support programme;

FIG. 23 provides a schematic to as a “Decision Tree,” showing how genomics and proteomics enable personalised treatment; and

FIG. 24A shows an outline exemplary decision matrix for use with embodiments of the invention.

FIGS. 24B to 24L show 24L filled out decision matrix information for 11 example patients.

FIG. 25 shows a flow chart for the smoking cessation embodiment. FTND is the Fagerstrom Test of Nicotine Dependence.

FIG. 26 provides a schematic to as a “Decision Tree,” showing how genomics and proteomics enable personalised treatment, linking dosage and bupropion ADR to genotype;

### DETAILED DESCRIPTION OF THE INVENTION

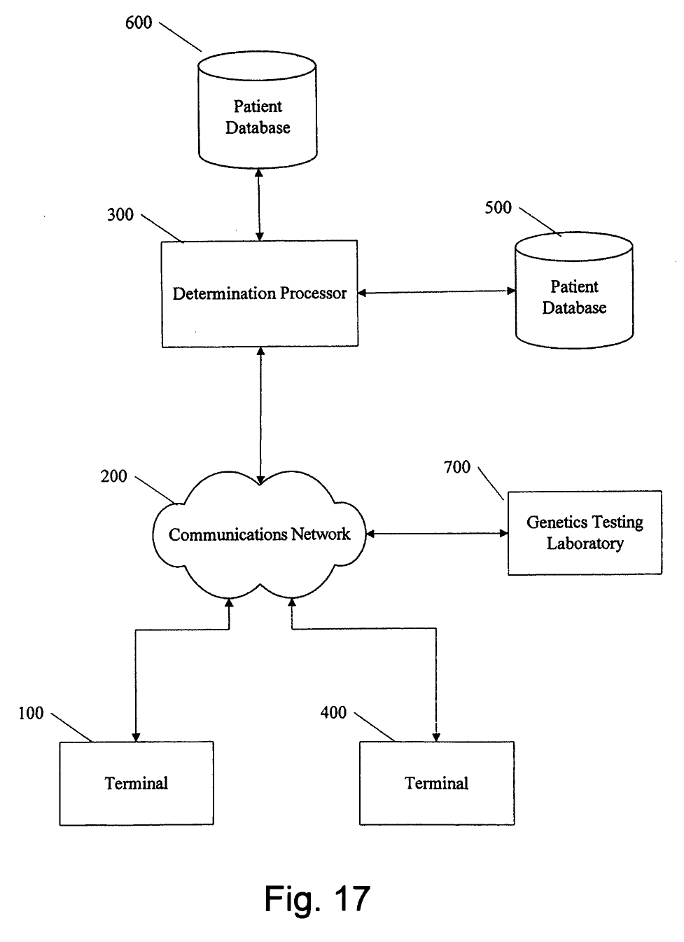

A generalised embodiment of the invention will now be described with reference to FIG. 17. This and other embodiments will be described with reference to a method and system for determining a therapeutic regime for a patient. For example, a user of such a system could be a patient with a medical condition seeking a personalised treatment program. In other situations a user could be a patient with a desire to modify an undesired behavioural habit.

These applications are by way of example only, and those skilled in the art will understand that the invention is not limited in this way. Embodiments of the invention could equally apply to the determination of a therapeutic regime for any purpose. It is equally the case that the invention is not limited in any way to the particular infrastructure mentioned below.

A system for determining a therapeutic regime to this generalised embodiment comprises a plurality of terminals connected to a communications network 200 via a suitable communications interface. A determination processor 300 is also connected to the network 200 via a suitable communications interface. Connected to the determination processor 300 is a patient database 500 and a determination database 600.

Also shown in FIG. 17 is a genetic testing facility 700. This is connected to the network 200 via a suitable communications interface. In other embodiments, it could be a genomic, proteomic, biochemical or metabolomic testing facility, or a testing facility for any suitable parameter relating to a patient.

In the generalised embodiment shown in FIG. 17 there are two terminals 100, 400. However, those skilled in the art will understand that the number of users in the system is not limited in any way, and that the number of terminals connected to the communications network could be very large. In addition, the system could comprise a single terminal. In the example of FIG. 17, terminal 100 is associated with patient A and terminal 400 is associated with doctor B.

In general terms, the method according an embodiment to the invention involves the following: the patient database 500 is arranged to store at least one of genomic, proteomic, biochemical or metabolomic information relating to a particular patient as well as personal information relating to that patient. The determination processor 300 is arranged to use this quantitative information and personal information in order to obtain at least one of genomic, proteomic, biochemical or metabolomic criteria and personal criteria personal criteria relevant to the therapeutic regime desired by the patient. The determination processor 300 then combines the or each genomic, proteomic, biochemical or metabolomic criteria with the personal criteria in order to determine a personalised therapeutic regime for the patient.

For the purposes of the rest of this description, reference will be made to genetic information and genetic criteria, however, it be appreciated that this could be any or combination of the following genomic, proteomic, biochemical or metabolomic information or genomic, proteomic, biochemical or metabolomic criteria.

In the embodiment of FIG. 17, the personal information of patient A is provided to the patient database 500 in the form of the results of a questionnaire filled in by the patient at their terminal 100. This questionnaire could include a series of questions relating to aspects of the patient's behaviour or habits that are relevant to the therapeutic regime desired. The personal information could also include information relating to the patient himself. For example, it could relate to the ethnicity of the patient, the patient's weight, height, body mass index results of analysis of blood for liver or renal function or any other characteristic of the user of the user's lifestyle that is relevant to the determination of the optimum therapeutic regime for the patient. The personal information obtained would be chosen in order to best fit with the therapeutic regime desired by the patient. It will be appreciated that in other embodiments, the personal information may be obtained in other ways, such as over the phone or in an interview.

Once completed, the results of the questionnaire could be sent from the terminal 100 to the patient database 500.

The unpopulated questionnaire could be provided by the determination processor 300 to the terminal 100 over the communications network. Alternatively, the questionnaire could be provided by another method, such as on a disk or CDROM to the user.

In the embodiment of FIG. 17, the genetic information of the patient is provided to the patient database 500 after analysis of a sample from the patient. The analysis could include DNA analysis, RNA analysis or any other method of genetic testing that is relevant to the determination of the optimum therapeutic regime for the patient. The genetic testing would be chosen in order to best fit with the therapeutic regime desired by the patient.

The genetic testing is performed at genetic testing facility 700 on a sample from the patient. The sample could be obtained by the patient himself with a suitable home test kit, or by a medical practitioner or otherwise. The sample would then be sent to the genetic testing facility 700 for analysis. The results of the analysis are sent to the determination database via the network 200. Alternatively, the results could be sent on a disk or CDROM on any other suitable delivery method.

The determination processor 300 is arranged to query the patient database 500 to obtain the personal information of the patient as well as the genetic information. The determination processor 300 obtains genetic criteria and personal criteria relevant to the therapeutic regime desired by the patient. This is done by comparing the genetic information and personal information of the patient with information stored in the determination database 600.

The determination database 600 stores a set of rules that are relevant to the therapeutic regime. For example, the determination database 600 could store information relating to which type of lifestyles (e.g. sedentary, active etc) are best suited to which therapeutic regimes. Furthermore, determination database 600 could store information relating to genetic information that indicates that one therapeutic regime would be preferable over one or more other therapeutic regimes.

The determination processor 300 then uses the information in the determination database 600 to obtain genetic criteria and personal criteria relevant to the therapeutic regime desired by the patient. The determination processor 300 then combines the genetic criteria with the personal criteria in order to determine a personalised therapeutic regime for the patient. This again is done by using information stored in the determination database 600. For example, the determination database 600 could store a set of rules that cause determination processor 300 to apply different weightings to different elements of the personal criteria and the genetic criteria. This could be done in the form of a decision matrix stored on determination database 600 and populated for the individual patient by the determination processor 300.

Once the personal criteria and the genetic criteria have been combined in this way, the determination processor 300 determines the personalised therapeutic regime for the patient. This personalised therapeutic regime is therefore highly tailored, because it takes into account both the patient's genetic makeup and information relating to the patient and the patient's lifestyle.

In this embodiment, the determination processor 300 then provides information relating to the personalised therapeutic regime via the network 200 to the terminal 400 of a doctor responsible for the patent. It will be appreciated, however, that this information could be provided to the doctor in other ways. The doctor would then provide any medication or medical advice to the patient required by the personalised therapeutic regime. Alternatively the determination processor 300 could provide the information relative to the personalised therapeutic regime via the network 200 to the terminal 100 of the patient, or some other content delivery method from the determination network to the patient. This would allow the patient to carry out the personalised therapeutic regime without the need to consult a doctor. This could be beneficial in circumstances in which the personalised therapeutic regime relates to medications available over the counter in a pharmacy or when the personalised therapeutic regime relates to a desired behavioural change in the patient.

In some embodiments, feedback from the patient is used to update the personalised therapeutic regime after it has been administered to the patient and during the course of the personalised therapeutic regime itself. This could be done by one or more additional questionnaires filled out by the patient or by another appropriate method. These additional questionnaires could relate to how the patient rates the effectiveness of the personalised therapeutic regime. This additional information could be provided to the determination processor 300 and could be used to determine an updated personalised therapeutic regime for the patient. For example, the determination process 300 could use the additional information to apply new weights to the genetic criteria and personal criteria stored in the determination database 600.

The feedback information from the patient currently undergoing the personalised therapeutic regime enables some embodiments of the invention to ensure that the personalised therapeutic regime is as accurate as possible for the patient. Such embodiments enable the success of the personalised therapeutic regime to be assessed and updated dynamically.

Furthermore, after the personalised therapeutic regime has been completed, some embodiments enable the results of the personalised therapeutic regime to be fed back into the system in order to improve therapeutic regime for new patients. For example, after the personalised therapeutic regime for patient A has been completed, a results questionnaire could be provided to patient A. By means of the questions in the questionnaire, the patient could rate the effectiveness of their personalised therapeutic regime. This results information could be fed back to the determination database 600, which could use it to update, for example, the weights applied to different genetic and/or personal criteria. This feeding back of results information enables the system according to such embodiments to grow in accuracy over time. It will be appreciated that other feedback methods are possible.

Alternatively, the patient's doctor could be the one assessing the results of the patient's personalised therapeutic regime, and the doctor could provide the results information to the determination database 600.

Embodiments of the invention that enable feeding back of information relating to the effectiveness of the personalised therapeutic regime, whether during or at the end of the personalised therapeutic regime, have the particular benefit that they enable personalised therapeutic regime for patients to be constantly tested and evaluated. This enables the information relating to the relevance of genetic criteria and/or personal criteria stored in the determination database 600 to grow both in size and accuracy over time.

This could happen both for an individual patient and at a group level for patients with similar illnesses and similar characteristics.

In the embodiment of FIG. 17, the terminal 100 could comprise a personal computer, a notebook computer, a laptop computer, a handheld computer, a workstation, a mainframe, a PDA or palmtop computer, a wearable computing device, a tablet, a smartcard, a device or product with embedded computing capability, a pager, a mobile telephone, an interactive television, or an application specific device. The personal information could be stored in a suitable data store comprised within the terminal 100. This data store could be a memory or physical storage means. Alternatively all or part of the personal information could be stored in a remote data store, and accessed by the terminal 100 when needed. In such a situation, the link could be achieved by using a local area network, wide area network or the Internet. The terminal can therefore be considered a sub-system in the system according to the generalised embodiment. Furthermore, as discussed, there is no requirement for the terminal to store the personal information, as it could simply be used to enable the patient to fill out the questionnaire.

The determination processor 300 could comprise a server running on one or more general purpose computers. The determination processor 300 and the determination database 600 and patient database 500 could together be considered a processor system, and be located on the same general purpose computer or computers. Alternatively, the determination database 600 and/or patient database 500 could comprise a number of data stores, located on one or more computers.

The communications network 200 could comprise any network whether it be a conventional landline network or a wireless network. More specifically, the communications network 200 could be provided by the Internet, an intranet, an extranet, a local area network, a wide area network or a network employing wireless application protocol. The wireless application protocol could comprise 808.11a, 808.11b, WAP, GRPS or 3G transmission. The components of the network can employ standard data networking protocols for data communications on the network 200 such as TCP/IP, HTML, XML, HTTP, DNS, LDAP.

The generalised embodiment discussed above has been discussed in relation to the communications infrastructure shown in FIG. 17 or alternatives thereto. However, it will be appreciated that this infrastructure is not required to perform the method according to the invention. For example, the patient does not require a terminal, and could fill out the required personal information using a paper questionnaire. Alternatively, such information could be provided by telephone. This information would then need to be entered into the patient database 500.

The present invention may be applied across a broad range of conditions, especially, but not restricted to, conditions or diseases that are strongly influenced by the interaction between the patients environment (for instance their lifestyle or where they live or work) and their genotype. Thus, the present invention is wide ranging in is application and is not limited to smoking, which is merely an embodiment of the present invention.

Indeed, any condition that may result form a genetic susceptibility is encompassed by the present invention. Such conditions may include cancer, obesity, autoimmune diseases such as hayfever and arthritis, heart disease and even AIDS the onset of which is known to occur at different times in otherwise similar individuals, blood pressure control, asthma, diabetes and other chronic diseases.

With regard to cancer, example conditions are breast cancer, prostate cancer, bowel cancer, testicular cancer and cancers of the blood, as well as lung cancer. Embodiments are the smoking cessation, depression treatment and influenza treatments.

With regard to depression, example conditions are Major Depression, Dysthymic Disorder, Unspecified Depression, Adjustment Disorder (with Depression) and Bipolar Depression.

Embodiments of the present invention can be used in conjunction with clinical diagnosis and testing, such as screening (for example for breast cancer as is common in women over 50), scans, such as MRI scans or body fluid testing (for instance blood tests), which may complement the online survey.

Methods of therapy for use in embodiments of the present invention include prescription or administration of drugs, gene therapy, and changes in lifestyle, either alone, or in any combination.

Any condition that results from a person's phenotype, the combination of their environment and their genotype, is envisaged. In one embodiment, the present invention relates to congenital diseases and the prediction and management thereof.

The process will also work if there is not a substantial genetic contribution to a particular disease. For instance, embodiments of the present invention can be used in conjunction with genomic, proteomic, and metabolomic data, or any combination thereof. The data for use in embodiments of the present invention can also be derived from using questionnaires.

It is understood that many conditions or aflicitions, such as smoking and obesity, are not necessarily viewed by all as diseases that need treatment. However, embodiments of the present invention merely seeks to provide a third party, from whom genetic information is received (for instance a patient), with information or medical advice on how to address such a condition.

Indeed, the present invention is not limited to treatments or therapies, but can equally be applied to the prophylaxis of conditions or the management of existing conditions.

In an embodiment, it is not even necessary for the medical advice be adhered to by the addressee or the patient, as according to one aspect, the present invention relates to the provision of tailored medical advice.

Methods of genetic analysis include those focusing on or including haplotyping, particularly at microsatellite loci, and genotyping, such as those methods identifying haplotypes or alleles or other genetic indicia. A genetic profile of the patient can be built of the patient, based on the genetic analysis conducted.

The genetic analysis may be a genotypic analysis, but can also be proteomic analysis, as this can still provide sufficient information to identify suitable therapies, for instance based on proteins with altered function or specificity. Accordingly, sources of genetic information are not only polynucleic acids, such as DNA and/or RNA, but it is also the case that amino acids or polypeptides can also be analysed to obtain information on the patient.

Suitable methods of genotyping and haplotyping are well know in the art and commercially available kits, for instance, are widely available. Alternatively, the skilled person could his won based on particular loci or SNP targets, such as microarrays bearing suitable complementary sequences or using the RT-PCR reaction for instance.

Reference to a patient is to be understood as any person to which the method of the present invention is to be applied, thus, it is not necessary that the patient has at the time that the method is used, a medically-diagnosed condition. It may be that they have a condition which is, as yet, undiagnosed, or that they are involved in the management or prophylaxis of a condition, whether diagnosed or not.

Non-limiting examples include, for instance, that the patient may be a smoker or had previously been a smoker. Similarly, the patient may be a depressive or may, as yet, not have been clinically diagnosed with depression. A further example is that the patient has flu, or is in an “at risk” group, such as the elderly or infirm.

The patient is a mammal, for example a human.

In a further aspect, embodiments of the present invention provides a kit, suitable for use in the preset method. The kit may comprise testing apparatus. The testing apparatus may comprise a device used to obtain genetic information from a sample and in some embodiments, analysing apparatus for identifying the genotype or haplotype, for instance, of the patient at least one loci identified as being relevant to the condition to be treated. The kit may also include the database and information processing system for the decision-making process. This may be provided as a self-contained or single kit for use in the field for instance. The kit may comprise a communication means for inputting the test result data into the rest of the analysis system.

The kit may comprise a microarray or a screening system for identifying genetic information as defined elsewhere.

### Smoking Cessation

The invention will now be exemplified, in a non-limiting manner, by reference to smoking and provision of medical technology to the cessation of smoking. This aspect of the invention makes use of a diagnostic tool that identifies a smoker's likely response to both nicotine and non-nicotine medications.

People smoke for many different reasons. Some smoke because of pressure from friends and relatives—others for relief of stress, from habit or addiction. Different people respond to the same drug in different ways so when the smoker wants to stop smoking it makes sense to take all these variables into account. The present process (herein after described as the “Nicotest™ process or programme”) works out the optimum way for the smoker to give up.

A first specific embodiment of the invention will now be discussed in relation to an Internet based method of determining a therapeutic regime for hitting a patient to seize smoking. This embodiment, also referred to as the Nicotest™, combines a DNA-based diagnostic test, questionnaire data, and with a comprehensive online support program. The method determines the best possible treatment for smokers to stop smoking and provides 24/7 online support moderated by specialists who reinforce the quit process.

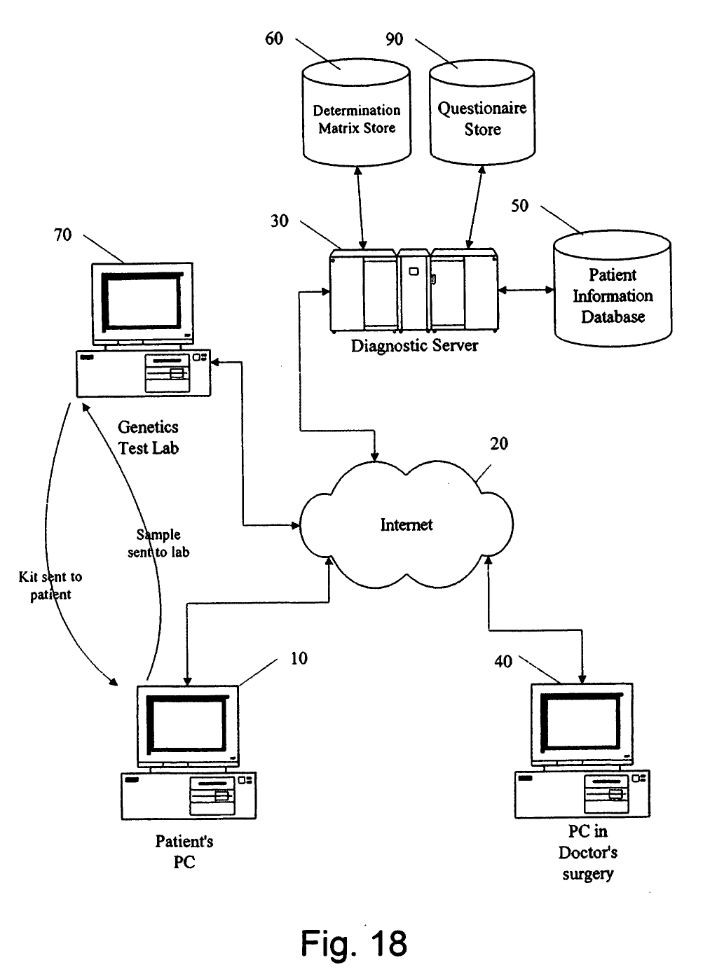

A system for implementing this method is shown in FIG. 18, which shows a patient's general purpose personal computer (PC) 10 connected to the internet 20. Also connected to the internet 20 is a diagnostic server 30, to which there is connected a decision matrix database 60 and a patient information database 50. Also shown in FIG. 18 is a genetic information testing lab 70 and a personal computer (PC) in a doctor's surgery 40. On this basis, it will be appreciated that all the elements in FIG. 18 can communicated to each other via the internet 20.

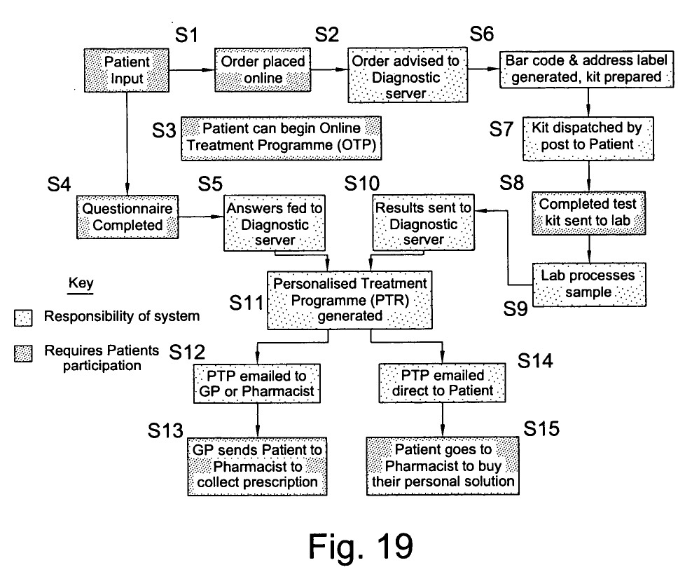

A flowchart showing a schematic representation of the method of this embodiment is shown in FIG. 19. The method starts with an input from the patient. The first step S1 is the placement of an order on the internet for the method. The order is divided to the diagnostic server 30 by means of the internet 20. It will be appreciated that there are numerous methods for placing orders online, and any appropriate method could be used.

After the order has been placed and the order advised to diagnostic server 30, the patient can begin the online treatment program according to this embodiment, as indicated in step S3.

Once the order is placed, the diagnostic server 30 generates a questionnaire to be sent to the patient from information in the questionnaire database 90. This questionnaire includes various information relating to the lifestyle of the patient and information related to the patient himself. In this embodiment the questionnaire includes questions relating to the number of cigarettes a day that the patent smokes, the patient's body mass index and the patient's ethnicity. The aim of the questionnaire is to obtain personal information from the patient that will be used to determine the personalised therapeutic regime for the patient. The personal information relates to information about the patient's life style and about the patient.

The diagnostic server 30 provides information relating to the questionnaire to the patients PC 10 by means of internet 20. For example, this could be in the form of an email attachment. This email attachment could be opened on the patients PC 10, and the questionnaire could be extracted therefrom.

At step S4, the questionnaire has been completed by the patient and at step S5 the answers of the questionnaire are fed to the diagnostic server 30. Again, this could be in the form of an email attachment, or alternatively by any suitable electronic delivery means.

As discussed above in relation to FIG. 17, in alternative embodiments the questionnaire could be paper based and once it has been generated it could be posted to the patient, who could complete it in writing and post it back for analysis.

In addition to generating the information relating to the questionnaire for the patient, after the diagnostic server 30 has been advised the patient has placed an order, a method also progresses to step S6.

In step S6, the diagnostic server 30 generates a barcode containing unique information relating to the patient, and an address label containing the patents address. This information is then sent to the genetic testing lab 70, which prepare a kit containing suitable apparatus for the patent to provide a sample for later DNA analysis. In this embodiment the kit is based on the SAFEspot™ blood sample collection and storage system discussed below. However in other embodiments, alternative sample collection means could be used. For example, the patient could travel to the genetic testing lab in person, in order for a sample to be obtained.

Furthermore, while in this embodiment the diagnostic server 30 prepares the barcode and address label for the kit to be sent to the patient, in other embodiments this could be prepared by the genetic testing lab 70 from information provided to it by the diagnostic server 30.

At step S7, the kit is dispatched by post to the patient. The patient then provides the single finger stick blood sample as discussed above in relation to the SAFEspot™ system discussed above.

At step S9, the genetic testing lab 70 processes and analysis the sample provided by the patient. In this embodiment the genetic testing lab 70 analysis two genetic predictors of response to treatments for stopping smoking. These are analysing whether the smoker has a common form of the gene that effect nicotine breakdown (CYP2A6) and analysing a gene that is known to affect the way the patient brain reacts to nicotine (DRD2). More details for genetic testing are discussed below.

The genetic testing lab 17 then sends the genetic information relating to the patient to the diagnostic server 30 by email. Alternatively it will be appreciated that other forms of communication of results could be possible.

At step S11, the diagnostic server 30 has now received the results of both the questionnaire information and their genetic information from the genetic testing lab 70. This information is stored in a patient information database 50, connected to the diagnostic server. The diagnostic server 30 uses this information from the patient information database 50 along with information in the decision matrix data store 60 in order to generate the personalised therapeutic regime for the patient. This is done on the basis of applying weightings to different genetic criteria and personal criteria and combining these by means of a decision matrix in order to determine the ultimate treatment regime for the patient.

In this embodiment, the treatment recommendations are reduced to four options: behavioural therapy; low dose nicotine replacement; high dose nicotine replacement and co-therapy (using two kinds of nicotine replacements simultaneously); and prescription of the drug bupropion.

As discussed above, the lifestyles information used to determined that the therapeutic regime are level of cigarette consumption, obesity and ethnic group. The genetic information used to determine the therapeutic regime is the presence or absence of a genetic marker of nicotine metabolic rate (CYP2A6) and dopamine D2 receptor status (ANKK1).

The general principles governing the choice of the therapeutic regime are in this embodiment:
People who smoke heavily are more likely to benefit from nicotine replacement therapy (NRT) and the higher the nicotine intake the greater the replacement needed.
Those with low nicotine metabolic rate need less replacement.
People with ANKK1 “T” tend to respond to NRT (Johnstone 2004)—those with “C” tend to respond to bupropion (LERMAN 2004).
People with obesity respond better to nicotine spray. Since nasal sprays alone are not currently recommended, these fall into the co-therapy group.
Treatments that use the minimum amounts of drugs are preferred.
Contra-indications to a drug therapy are either a responsibility of the pharmacist or doctor.
Suggestions of those under 18 or pregnant are based on the usual inputs, but extra advice is given for them to see their doctor for treatment.

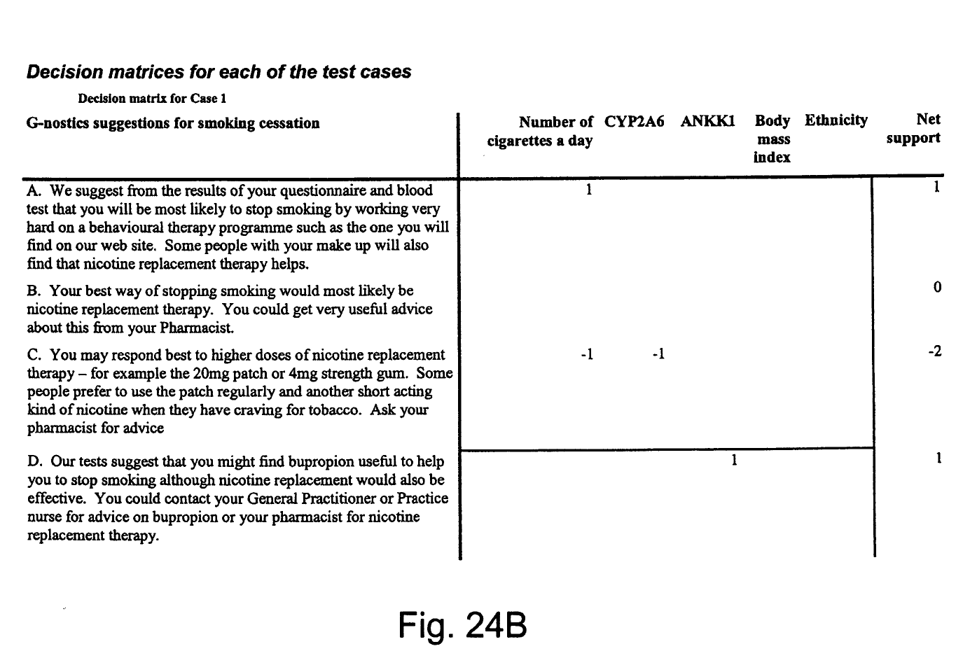

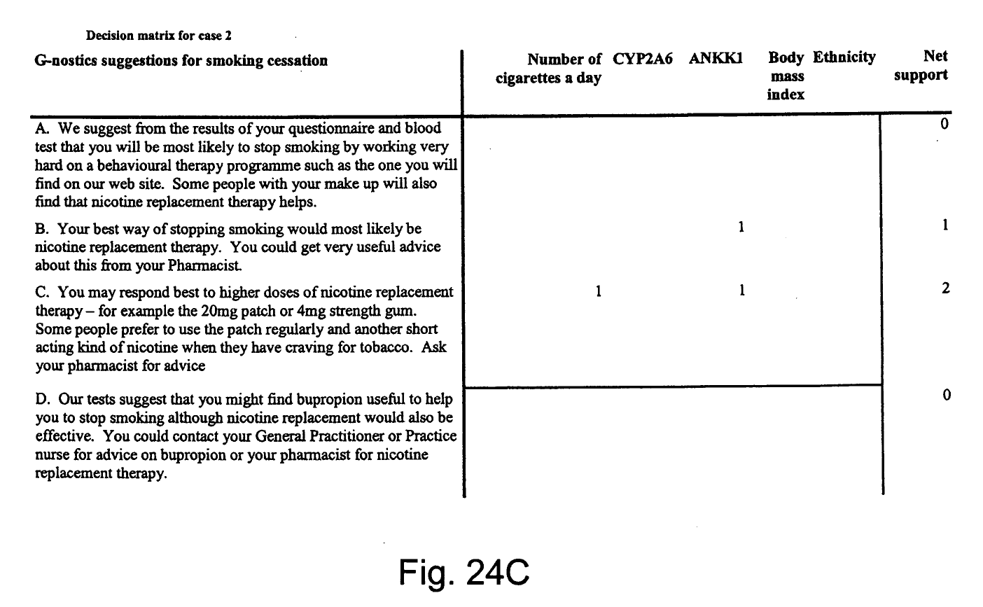

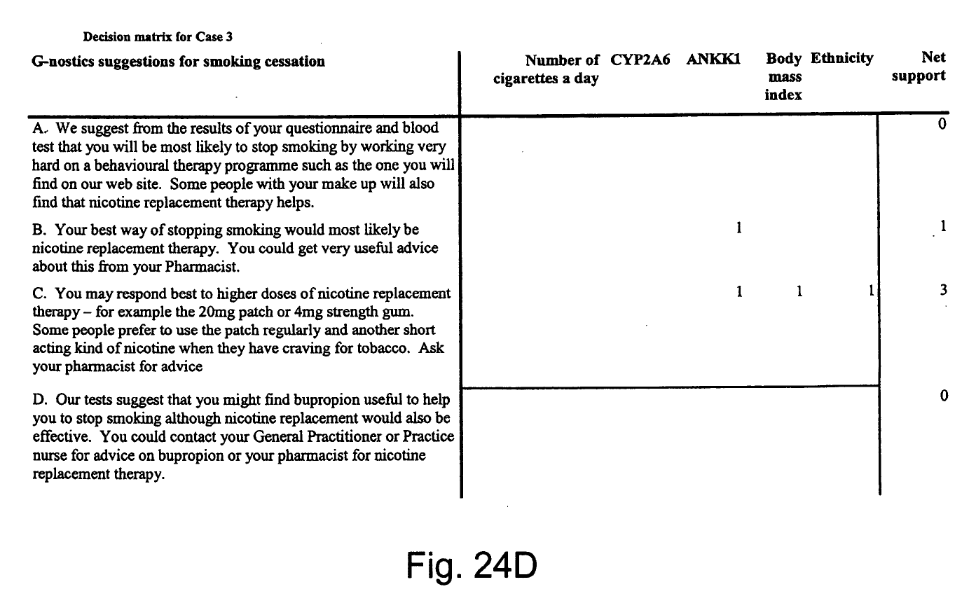

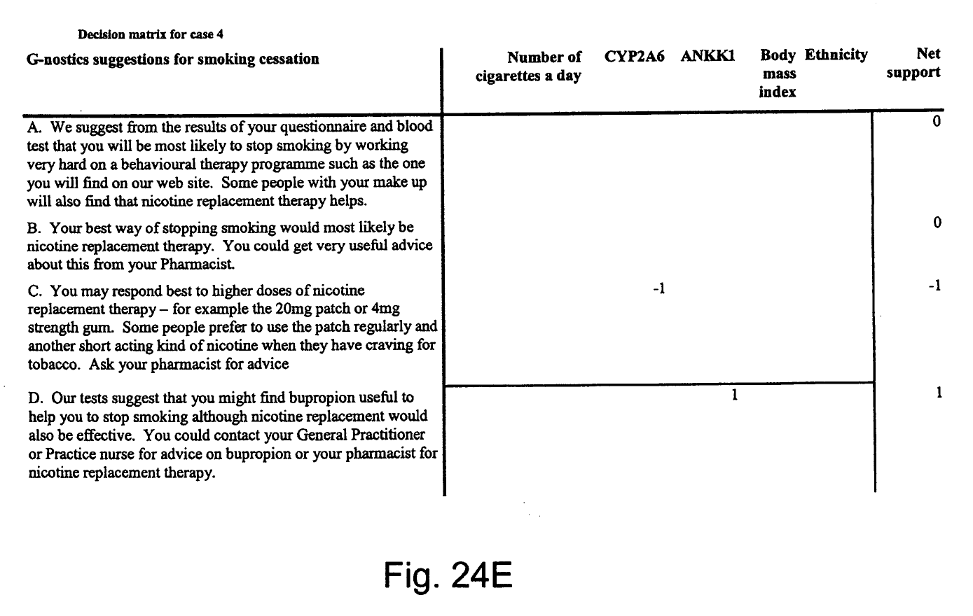

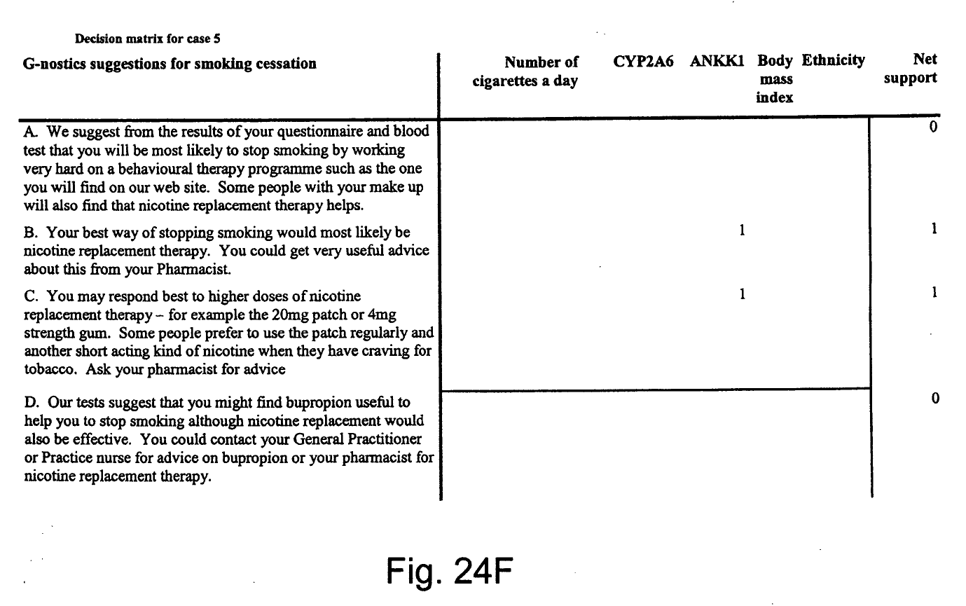

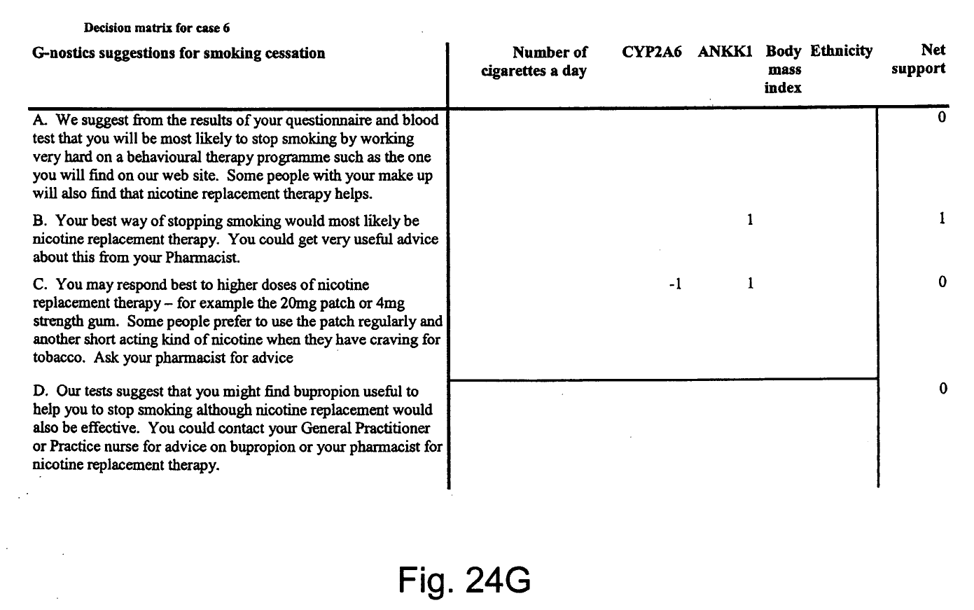

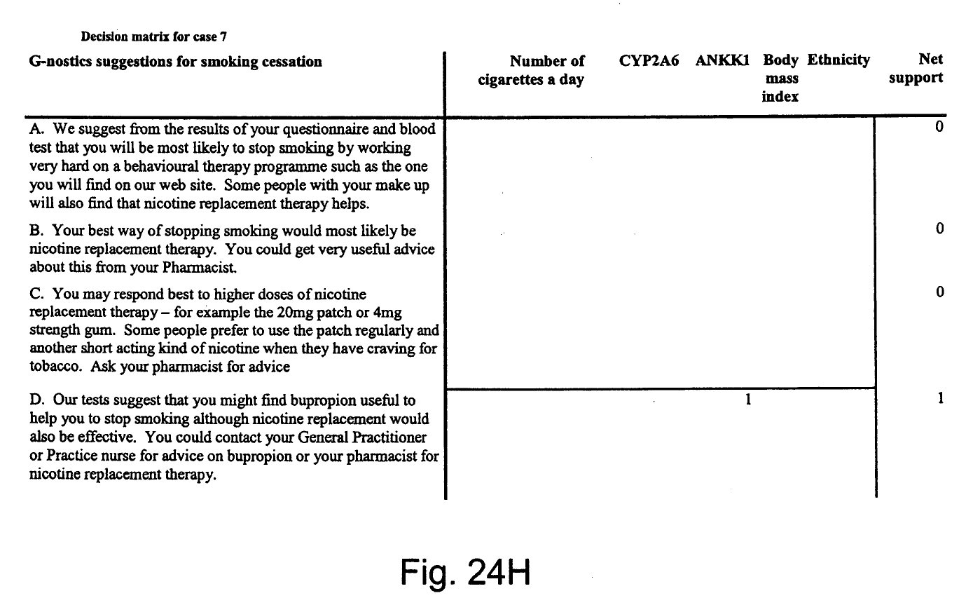

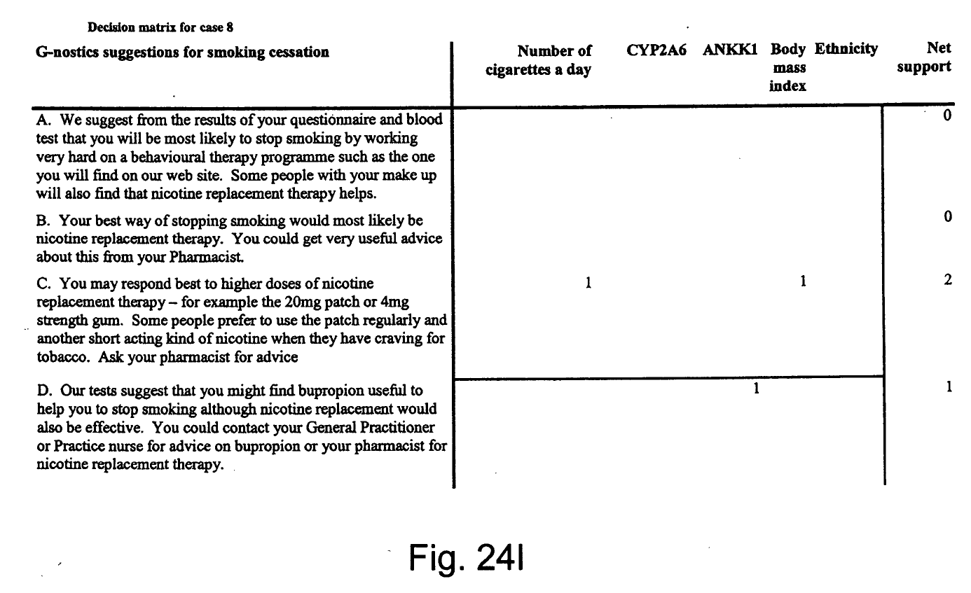

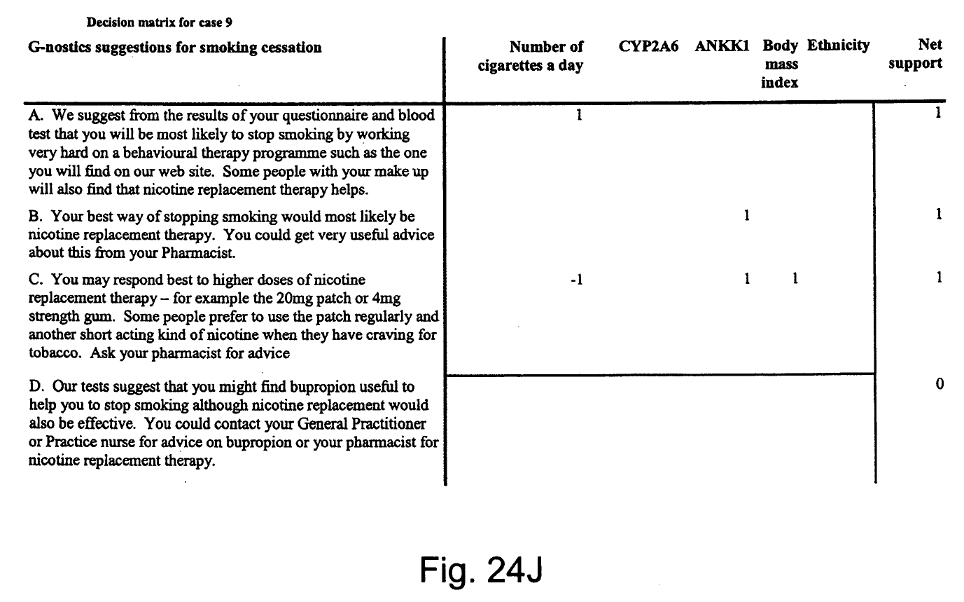

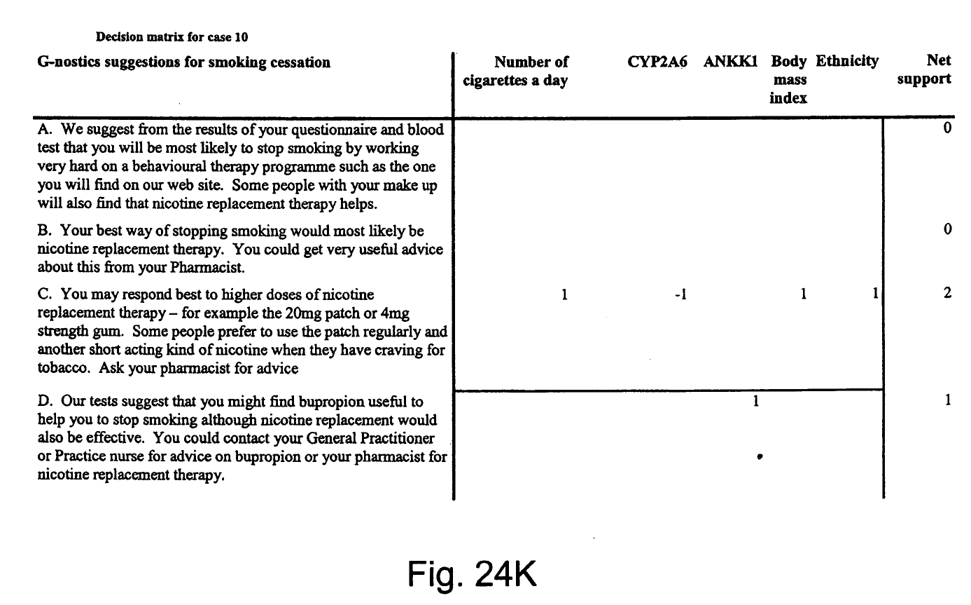

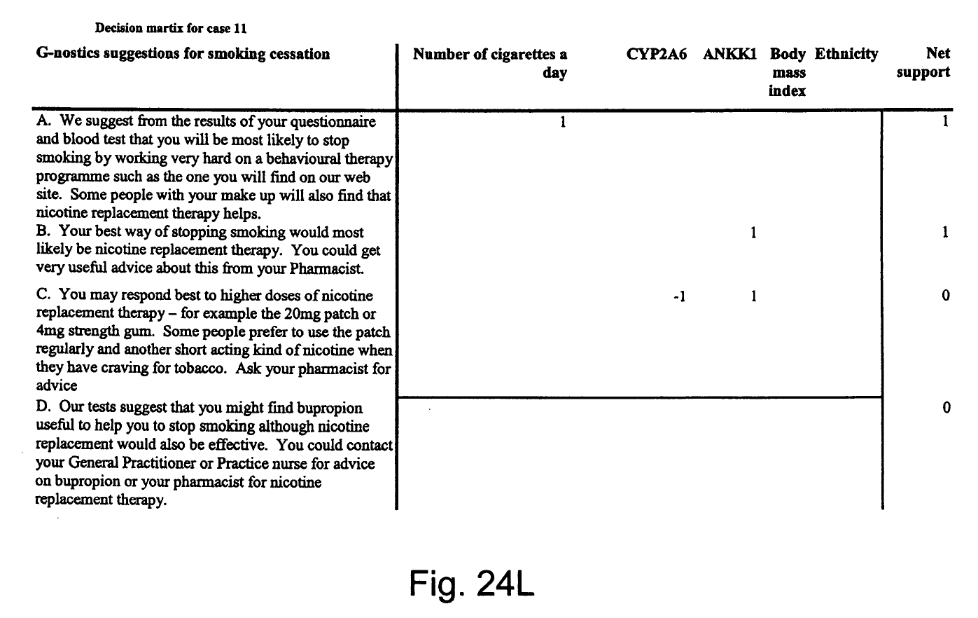

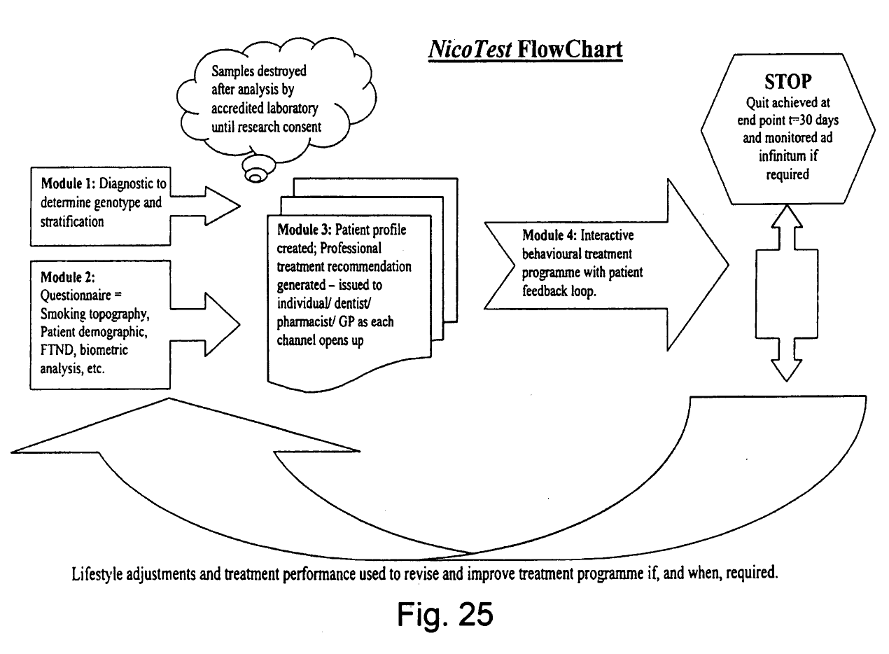

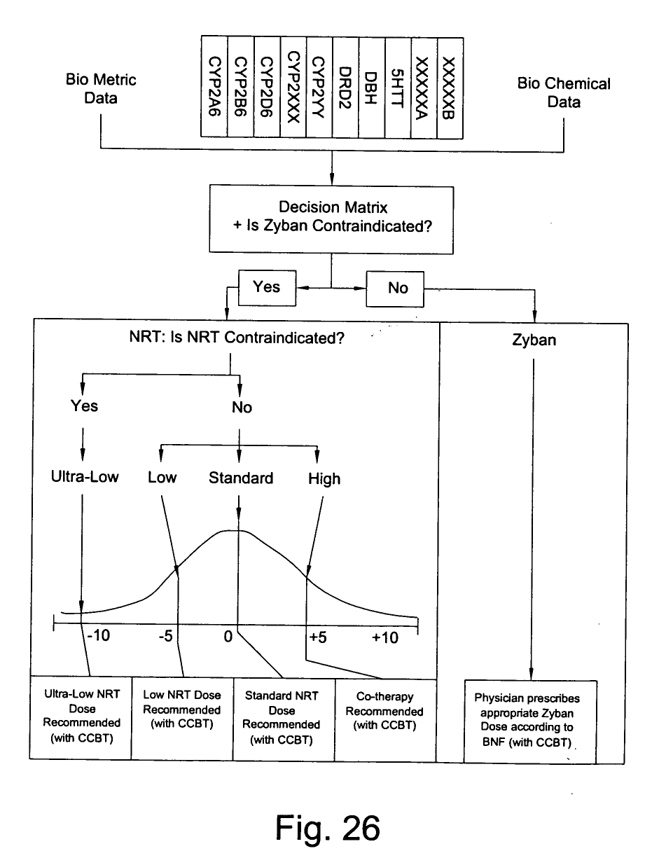

These principles are built into the decision matrix shown in FIG. 24A. The net support for each output (recommendation) arises from simple addition of weights in each cell.

The rules for completing the outline matrix can be summarised as follows:

### Rules for Completing the Outline Matrix

Number of Cigarettes a Day

If >20 let option C=1
If <10 C=−1, A=1
   CYP2A6
If A6=null C=−1
   ANKK1
If ANKK1=‘T’ let options B and C=1
If ANKK1=‘C’ let option D=1
   BMI
If BMI >30 let option C=1
   Ethnicity
If ethnicity is nonCauc let option C=1
In case of a tie give A preference over B, over C, over D

Program will also say ‘These suggestions assume that there are no medical reasons that prevent you from taking particular drugs—please ask your pharmacist for advice.’

And

‘If you are under the age of 18 or pregnant you will need to see a General Practitioner for any medicines.’

In order to provide a number of examples, FIGS. 24B 24L provide filled out decision matrix information for 11 example patients. The details of example patients are as follows:

### Test Cases with Recommendations

1. 43 year old Caucasian woman with normal BMI smoking 8 a day who is a slow metaboliser of nicotine and ANKK1 ‘C’. A
2. 16 year old male, normal BMI, smoking 20 a day, normal metaboliser of nicotine and ANKK1 ‘T’. C
3. 40 year old Afro Caribbean, high BMI, smoking 15 a day, normal nicotine metaboliser, ANKK1 ‘T’. C
4. 50 year old man with normal BMI smoking 15 a day who is a slow metaboliser of nicotine and ANKK1 ‘C’. D
5. 35 year old woman with normal BMI smoking 15 a day, normal nicotine metaboliser, ANKK ‘T’ B
6. 20 year old man with normal BMI smoking 12 a day, slow metaboliser of nicotine with ANKK1 ‘T’. B
7. 49 year old man with normal BMI smoking 15 a day, normal nicotine metabolism, ANKK1 ‘C’. D
8. 30 year old woman with BMI >30, smoking 25 a day, normal nicotine metaboliser and ANKK1 ‘C’ C
9. 25 year old man with BMI >30, smoking 5 a day, normal nicotine metabolism, and ANKK1 ‘T’. A
10. 30 year old woman with BMI >30, smoking 24 cigarettes a day, slow nicotine metaboliser, ANKK1 ‘C’. C
11. 45 year old man with normal BMI smoking 6 cigarettes a day, slow nicotine metaboliser and ANKK1 ‘T’. A

The above test cases are provided with the recommendation (either A, B, C or D). The examples of completed decision matrix is for test cases 1 to 11 are provided in FIGS. 24B to 24L.

Once the personalised therapeutic regime has been generated for the patient, it can either be emailed to a doctor (for example a general practitioner GP or a pharmacist). In step S13, the doctor, for example a GP would send the patient to a pharmacist to collect a prescription for the required medication. The results could be sent to the doctor's surgery by means of email to the doctors PC 40. Alternatively, other ** mechanisms could be used.

In other embodiments, the illustrated in steps S14 and S15, personalised treatment regime could be emailed directly to the patients PC 10. The patient would then be responsible for going to the pharmacy to but their personal solution.

In some embodiments, feedback from the patient is used to update the personalised therapeutic regime after it has been administered to the patient and during the course of the personalised therapeutic regime itself. This could be done by one or more additional questionnaires filled out by the patient. These additional questionnaires could relate to how the patient rates the effectiveness of the personalised therapeutic regime. This additional information could be provided to the diagnostic processor 30 and could be used to determine an updated personalised therapeutic regime for the patient. For example, the diagnostic processor 30 could use the additional information to apply new weights to the genetic criteria and personal criteria stored in the decision matrix database 60.

The feedback information from the patient currently undergoing the personalised therapeutic regime enables embodiments of the invention to ensure that the personalised therapeutic regime is as accurate as possible for the patient. Such embodiments enable the success of the personalised therapeutic regime to be assessed and updated dynamically.

Furthermore, after the personalised therapeutic regime has been completed, some embodiments enable the results of the personalised therapeutic regime to be fed back into the system in order to improve therapeutic regime for new patients. For example, after the personalised therapeutic regime for patient A has been completed, a results questionnaire could be provided to patient A. By means of the questions in the questionnaire, the patient could rate the effectiveness of their personalised therapeutic regime. This results information could be fed back to the decision matrix database 60, which could use it to update, for example, the weights applied to different genetic and/or personal criteria. This feeding back of results information enables the system according to such embodiments to grow in accuracy over time.

Alternatively, the patient's doctor could be the one assessing the results of the patient's personalised therapeutic regime, and the doctor could provide the results information to the decision matrix 60.

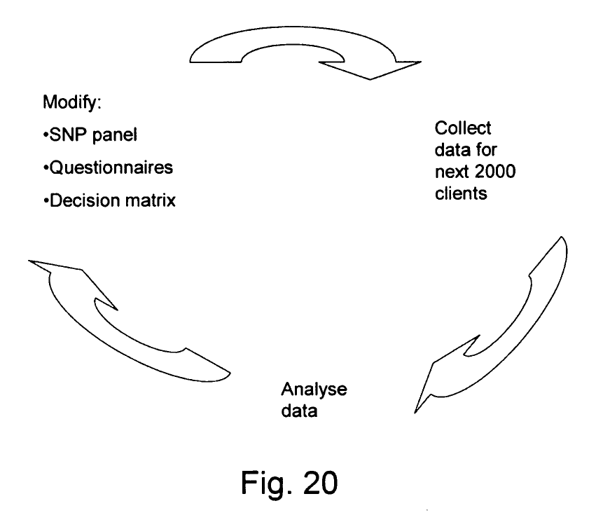

Embodiments of the invention that enable feeding back of information relating to the effectiveness of the personalised therapeutic regime, whether during or at the end of the personalised therapeutic regime, have the particular benefit that it enables personalised therapeutic regime for patients to be constantly tested and evaluated. This enables the information relating to the relevance of genetic criteria and/or personal criteria stored in the decision matrix 60 to grow both in size and accuracy over time. This is illustrated in FIG. 20.

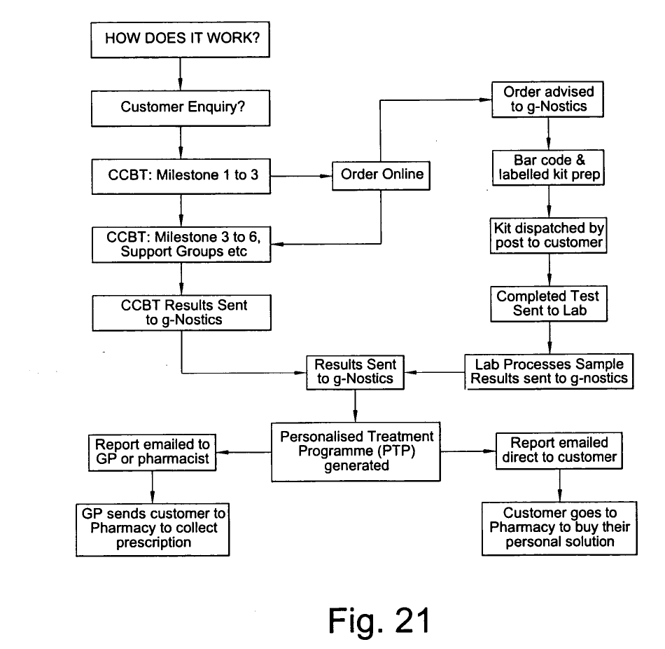

A specific flowchart relating to the NICOTEST™ is shown in FIG. 21. This combines the personal therapeutic regime discussed above with a number of interactive, moderated on line support program features. There are two main components to the NICOTEST method.

### There are Two Main Components to the NicoTest™ Solution

1. Diagnosis

An online lifestyle and smoking history questionnaire (Milestone 1, 2 and 3) determines the smoker's level of nicotine dependence and identifies appropriate lifestyle changes that may be needed. This phase includes the well known Fagerström Test of Nicotine Dependence (FTND)
A diagnostic tool that identifies a smoker's likely response to both nicotine and non-nicotine medications
A Personalised Treatment Report that recommends the most appropriate treatment for each patient
   2. Online Support Programme (The Flight to Freedom)
An 11-week course of motivational emails commence after registration
A professionally moderated, interactive, online support programme that encourages behavioural change. Our online support programme is available 24/7 and includes:
   A QuitMeter that monitors the smokers nicotine dependence, financial savings and life years gained as they progress
   Coping plans to minimise weight gain, improve fitness and reduce stress
   A chat room to meet ‘Buddies’, Internet Messenger to communicate with peers and interactive exercises to help the smoker prepare and adapt to their new smoke-free lifestyle
   Interactive activities to reinforce and maintain the quit process
   Follow-up survey to self-report progress and confirm effectiveness of treatment recommendations

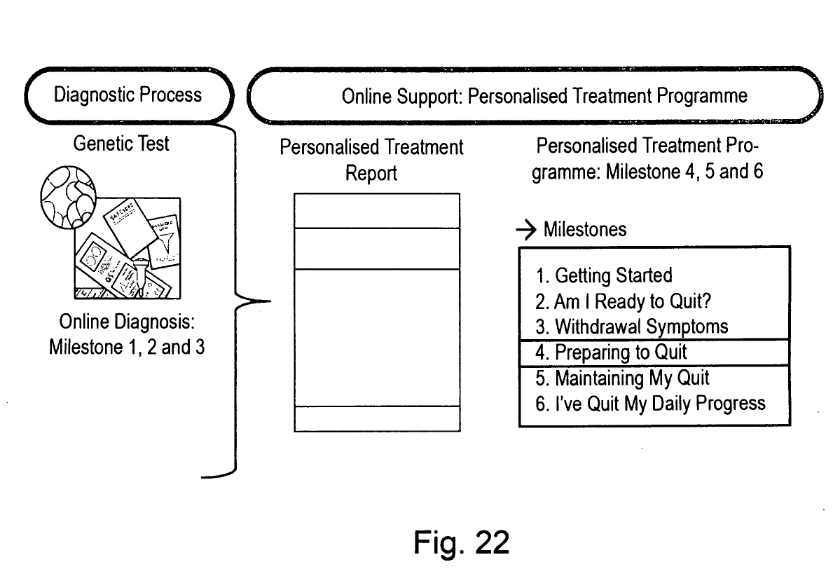

The above is shown schematically in FIG. 22.

The NicoTest™ programme therefore starts with a series of questions to determine the smoker's smoking topography and demographics. These questions establish the smoking history and help determine why the smoker smokes. Coupled with the results from the DNA analysis this information is used to find out the best way for them to stop smoking. The Nicotest™ provides a ‘personalised’ programme that is specific to the individual to help smokers quit. For example, smokers can log in to a 24-hour support service on the Internet that will help them prepare and adapt to their new smoke-free lifestyle. In addition, after the personalised therapeutic regime has been delivered, the method involves providing a Cognitive Behavioural Therapy program to the patient. This uses a metaphor which called the Flight to Freedom and breaks the quit process into six Milestones. Registrants need to complete the DNA test and complete Milestone 1, 2 and 3 before a Personalised Treatment Report is issued.

Pharmacogenetics

It is thought that some differences in smoking habits come from inherited changes in the way the body reacts to nicotine. Differences in our genes mean that some people break down nicotine more quickly than others. This means that the nicotine does not last long in the bloodstream. The smoker may, therefore, benefit from higher levels of nicotine replacement. It is thought that heavier smokers often benefit from higher doses of NRT (Nicotine Replacement Therapy) when they are trying to quit as they need to replace higher levels of background nicotine.

Smokers are told whether they have the common form of a gene that affects nicotine breakdown (known as CYP2A6), which may also be used to determine level of replacement.

The method also lets the healthcare professional and their smoker patients know about another gene that may affect the way their brain reacts to nicotine (known as DRD2). It is thought that one form of the gene (for instance a particular allele) is more common in people who smoke regularly. Some studies suggest that people with different forms of the gene do better with different treatments (bupropion vs nicotine replacement). This research is at an early stage and these results are being checked in further studies. There will be much more known about this in a few years time. In the meantime, it is believed that smokers will find it interesting to know what form of the gene they have. This might help them understand what makes them smoke and work out how best to stop with different types of medication. This physiological aspect is one of the benefits of the present invention.

It will be understood, of course, that this genetic information can also be used to identify appropriate treatments, medications, therapies and regimes, allowing the user, for instance the operator or healthcare professional, to tailor the therapy to the patient, for instance the smoker.

In relation to the smoking embodiment of the present invention, it will be appreciated that there are a wide range of smoking-related genes that are known in the art, some directly related to nicotine dependence through the ability to break down nicotine, others more indirectly through pleasure or reward pathways. As the processing of the human genome continues, more genes ad pathways associated with smoking addiction, susceptibility and cessation will no doubt become apparent.

Indicator variables for five genetic markers are particularly preferred. Specifically, the CYP2A6 gene, the CYP2B6 gene, preferably any of combinations CC, CT, TT; DRD2 allele, preferably any of combinations A1A1, A1A2, A2A2; TPH allele, preferably any of combinations AA, AT, and TATA; and DAT, preferably any of the three allele combinations of the dopamine transporter gene (DAT). DAT is a gene similar to DRD2 in that it affects the process of dopamine uptake into cells.

In understanding the role of genetics in the development of smoking addiction, two strands of evidence must be considered. First, there is the question of whether there is a genetic component to these complex behaviours. Second, specific candidate genes must be identified that, depending on their composition, have a significant impact on health behaviour. In terms of smoking behaviour, the traditional method of considering genetic linkages, twin studies have provided overwhelming evidence of a genetic effect on smoking. Fisher, followed by numerous researchers over the years including Carmelli (1990), Sullivan (2001), and others, have estimated between 46-84% inherited component when comparing monozygotic to dizygotic twins. While recent papers have shown the robustness of such results could be due in part to different parenting strategies for mono vs dizygotic twins, it is highly unlikely that such effects could explain away all of the effect, seen on all continents.

For the second question, five candidate genes have been shown to be particularly promising, the TPH locus, DBH locus, the DAT locus, the CYP gene loci and the Dopamine Receptor (DRD2) ANKK1 locus. The TPH locus, which relates to the formation of the important neurotransmitter serotonin, has been implicated in an earlier age of onset of smoking (Lerman, 2001) as well as a higher rate of experimentation among youth (Sullivan, 2002). The second candidate loci gene, CYP, codes for the Cytochrome P450 group of enzymes, that are involved in metabolizing substances such as nicotine in the liver. Pianezza (1998) found that specific alleles (each person inherits two allele types at each genetic marker) associated with decreased nicotine metabolism were found in smokers versus non-smokers. Other studies found that smokers with other CYP alleles smoked fewer cigarettes each day and were more likely to succeed in quitting.

The fourth promising candidate genes are the DRD2 and DAT, especially the DAT1, loci, which codes for dopamine receptors and transporters, respectively (the dopaminergic pathways in the brain are believed to have an important role in the development of nicotine addiction). For the former, Nobel et. al. (1999) found that there was a higher prevalence of a specific DRD2 allele among smokers while Lerman et. al. (1999) found 49% of Caucasian smokers carrying that same allele as compared to 26% of non-smokers.

Also preferred are genetic markers or indicia found on the human dopamine beta-hydroxylase gene, DBH, preferably the 444 G and/or A alleles, the 910 ploymorphism, 1368 A and/or G alleles, the 5-HTT serotonin transporter, CYP2D6 gene and the human monoamine oxidase A (MAO A) gene, preferably the 1460 C allele. Also preferred are alleles or polymorphisms in linkage disequilibrium with the alleles or polymorphisms of the present invention.

Suitable genes will be known to the skilled persona and are as described in the accompanying references and WO 01/38567 (Isis Innovation Limited), all of which are hereby incorporated by reference.

As mentioned previously, the genetic component to smoking behaviour is likely to be multifactorial with different molecular mechanisms contributing to the habit in different people. The dopamine metabolic genes analysed in this study are likely to form only part of the genetic component of nicotine addiction. Polymorphisms in dopamine receptors D1 and D2 are likely to contribute, although not all studies confirm these associations. The gene for the D4 receptor has a variable number tandem repeat polymorphism in which the 7 repeat allele reduces the affinity of the receptor for dopamine. This polymorphism seems to predispose to smoking only in African Americans in whom it is more frequent (40%) than in Caucasians.

Another important protein is the dopamine transporter (DAT 1) which is responsible for removing dopamine from the synaptic cleft and thereby terminating its action. A variable number tandem repeat occurs in the 3′untranslated region of this gene. The 9 repeat allele, of uncertain functional significance, is associated with a reduced likelihood of being a smoker. Smokers who have this allele start smoking later and have longer periods of abstinence than those with fewer repeat sequences. The effects of the dopamine receptor and transporter polymorphisms on smoking may be mediated by an association with a novelty seeking personality.

In view of the foregoing, it is within the scope of the invention to performs screens for the presence or absence in the genome of the human subject of at least one allele selected from the group consisting of: dopamine P-hydroxylase 1368 A, dopaminehydroxylase 1368 G, monoamine oxidase A 1460 C and monoamine oxidase 1460 T in conjunction with screens (in the same human subject) for other polymorphisms associated with smoking behaviour, for example as part of a panel of screens. In a preferred embodiment, the panel of screens would include up to 10 different polymorphisms.

The individual screens to be included in the panel may be selected from the group consisting of: screens for DBH 1368 A and/or one or more alleles in close physical proximity to or in linkage disequilibrium with DBH 1368 A, screens for MAO A 1460 C and/or one or more alleles in close physical proximity to or in linkage disequlilibrium with MAO A 1460 C, screens for one or more alleles of the dopamine D1 receptor Dde1 RFLP or one or more alleles in linkage disequilibrium therewith, screens for one or more alleles of the dopamine D2 receptorTaq1 RFLP or one or more alleles in linkage disequilibrium therewith, screens for one or more alleles of the dopamine D4 receptor VNTR polymorphism or one or more alleles in linkage disequilibrium therewith, screens for one or more alleles of the VNTR in the 3′ UTR of the DAT 1 gene or one or more alleles in linkage disequilibrium therewith, screens for one or more alleles of the CYP1A1 MsI RFLP or one or more alleles in linkage disequilibrium therewith and screens for one or more variant alleles of CYP2A6, CYP2D6, the tyrosine hydroxylase gene (TH) or the 5-hydroxytryptamine transporter gene (5-HTT).

The step of screening for the presence or absence of specific alleles, also referred to herein as ‘genotyping’, can be carried out using any of the methodologies known in the art.

Genotyping of single nucleotide polymorphism (SNPs) may be carried out by performing PCR using allele specific primers, a technique known in the art of PCR-SSP. Further techniques are known in the art for the scoring of SNPs (see review by Schafer, A. J. and Hawkins, J. R. in Nature Biotechnology, Vol 16, pp33-39 (1998), including mass spectrometry, particularly matrix-assisted laser adsorption/ionisation time-of flight mass spectrometry (MALDI-TOF-MS, see Roskey, M. T. et al., 1996, PNAS USA, 93: 4724-4729), single nucleotide primer extension (Shumaker, J. M et al., 1996, Hum. Mutat., 7:346-354; Pastinen, T. et al., 1997, Genome Res., 7:606-614) and DNA microchips/microarrays (Underhill, P. A et al., 1996, PNAS USA, 93:196-200). The known techniques for scoring polymorphisms are of general applicability and it would therefore be readily apparent to persons skilled in the art that the known techniques could be adapted for the scoring of single nucleotide polymorphisms in the monoamine oxidase A gene and the dopamine β-hydroxylase gene.

Variable number tandem repeat polymorphisms, such as the MAO A uVNTR, can be scored by performing non-allele-specific PCR using primers corresponding to sequences on either side of the variable number repeat region. Different alleles will give rise to PCR products of slightly different sizes which may be resolved by gel electrophoresis or other techniques known in the art.

Restriction fragment length polymorphisms are typically scored by digesting genomic DNA with the appropriate enzyme then performing a Southern blot using a labelled probe corresponding to the polymorphic region (see Molecular Cloning: A Laboratory Manual, Sambrook, Fritsch and Maniatis, Cold Spring Harbor Laboratory, NY).

Suitable kits are described in WO 0138567, as are suitable oligonucleotide molecules for inclusion into the kit, which are preferably single stranded and may correspond to the sense strand or the antisense strand of the relevant gene and to either allelic variant. In particular, oligonucleotides suitable for performing PCR-SSP genotyping of the MAO 1460 C/T and the DBH 1368 A/G polymorphisms, are disclosed therein.

WO 0138567, incorporate herein by reference, discloses an association between genetic variation in the dopamine β-hydroxylase gene and tobacco consumption, and identifies the dopamine β-hydroxylase enzyme as a target for pharmaceutical intervention in the development of treatments/therapies to assist in smoking cessation. It was shown that inhibitors of dopamine β-hydroxylase are likely to ameliorate the withdrawal effects of nicotine.

Polymorphisms in the dopamine D-2 receptor ((32806)DRD2 C/T and (22316)DRD2 A/G) and in dopamine beta hydroxylase ((1368)DBHA/G) have been implicated in modulation of smoking and other reward-seeking behaviours. It had been hypothesized (Johnstone E C; Yudkin P L; Hey K; Roberts S J; Welch S J; Murphy M F et al. Genetic variation in dopaminergic pathways and short-term effectiveness of the nicotine patch. Phannacogenetics 14(2): 83-90, 2004) that these alleles would predict the outcome of nicotine patch therapy for smoking cessation. In 1991-93, the above authors performed a randomized controlled trial of the nicotine patch on 1686 heavy smokers (greater than or equal to 15 cigarettes/day). In 1999-2000, they contacted 1532 of the 1612 subjects still available; 767 (50%) completed a questionnaire and gave a blood sample. In the 755 cases in which DNA was successfully genotyped, the authors examined associations between the polymorphisms in DRD2 and DBH, and smoking cessation. At 1 week, the patch was more effective for smokers with (32806) DRD2 CT/TT genotype [patch/placebo odds ratio (OR) 2.8, 95% confidence interval (CO 1.7-4.61 than with CC (OR 1.4, 0.9-2.1; P for difference in ORs 0.04). Smokers with both (32806) DRD2 CT/TT and (DBH)-D-1368 GA/AA genotypes had an OR of 3.6 (2.0-6.5) compared to 1.4 (1.0-2.1) for others (P=0.01). At 12 weeks, the ORs for these genotypic groups were 3.6 (1.7-7.8) and 1.4 (0.9-2.3), respectively (P=0.04). There was no association between patch effectiveness and (22316) DRD2 exon 8. Short-term effectiveness of the nicotine patch may be related to dopamine beta-hydroxylase and dopamine D2 receptor genotype. The DRD2 (32806) CT/TT and DBH (1368) GA/AA genotypes are, therefore, preferred targets for genetic analysis according to the smoking embodiment of the present invention, whether alone, or in combination.

Indeed, it will be appreciated that any of the preferred genes, loci, haplotypes or target for genetic analysis according to the present invention may, preferably, be taken alone. It is, however, preferred that they are analysed in combination to build a genetic profile of the patient that can be used according to the present invention, for instance by being correlated to data and thereby influencing decision-making process to a greater degree.

It will be appreciated that such traits are likely to have a polygenic nature. There may also be a degree of population stratification, commonly referred to as genetic admixture, in which different alleles pool in different ethnic groups, thus raising endogeneity, or confounding, concerns. See Singleton 1998, Wacholder, 2002, Lerman, 2001 and the references within for an extensive discussion. The basic idea is that genetic alleles are pooled among certain ethnic groups, thus causing difficulty in a specification strategy Wacholder (2002) provides a comprehensive list of criteria that must be fulfilled in order to claim bias from genetic admixture. However, the skilled person will be aware of such issues and these will, preferably, be factored into the analysis and decision-making process.

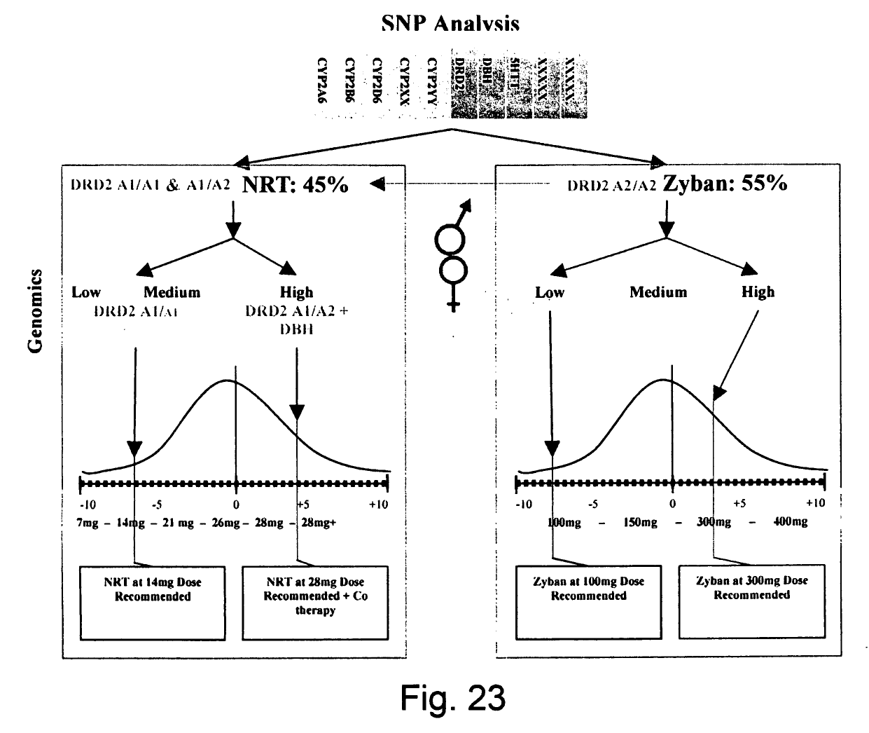

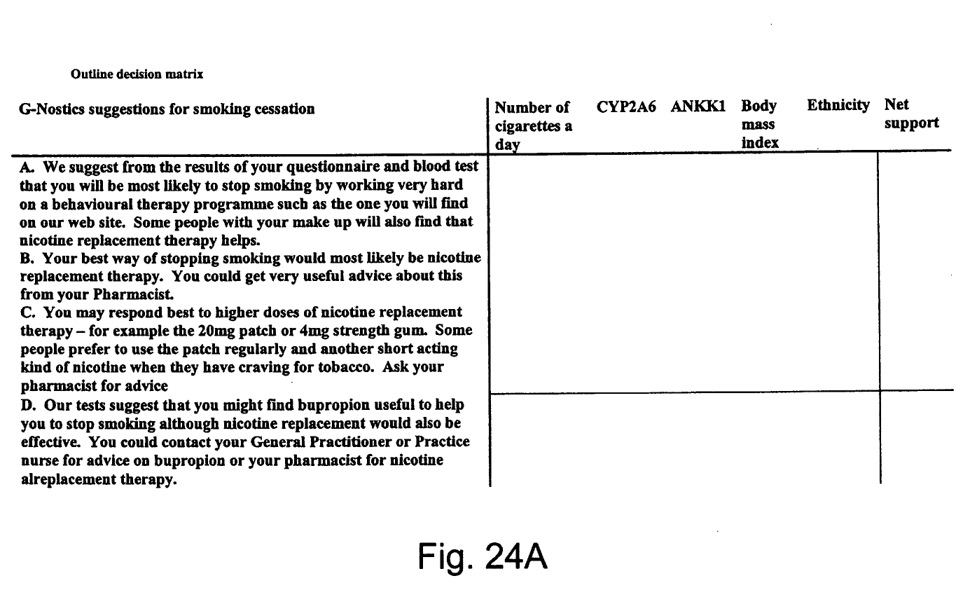

FIG. 23 provides a schematic to as a “Decision Tree,” showing how genomics and proteomics enable personalised treatment.

It is noteworthy that 41% of women carry DRD A1 gene variant so they are more likely to suffer side effects from Zyban. Men may benefit more from Zyban.

This schematic shows how, having conducted an analysis of a smoker's genetic information, for instance, a Single Nucleotide Polymorphism (SNP) analysis of the DRD2 gene, focusing on the A1/A2 allele. Smokers that have at least one copy of the A1 allele in their genome (i.e. those were that were A1 homozygous (A1-A1) or heterozygous (A1-A2)) were found to comprise 45% of the tested sample of smokers and were prescribed a course of NRT. Smokers that were A2 homozygous (A2-A2) were found to comprise 55% of the tested sample of smokers and were prescribed a course of Zyban.

Table 13 entitled “Smoking Cessation Evidence” shows the results of the present invention in effect.

TABLE 13
Smoking Cessation Evidence

| | Increase in | |
|---|---|---|
| | percentage of | |
| | smokers | Best |
| | abstaining for 6 | Quit |
| Intervention element | months or longer | Ratio |
| 3 minutes very brief advice by clinician | 2% | 1:50 |
| Up to 10 minutes, very brief advice by clinician | 3% | 1:33 |
| Intensive support only (Smoking Cessation | 7% | 1:14 |
| Service) | | |
| NRT added to brief advice plus/minus placebo | 5-8% | 1:12 |
| Intensive behavioural support (Smoking | 13-19% | 1:5 |
| Cessation Service) plus NRT or Zyban | | |
| Personalised Treatment Programme (PTP) | 20%-40% | 1:2.5 |
| according to the present invention | | |

In particular, it can be seen that the there is a substantial increase in the Quit ratio of 1:2.5, which is approximately double that of the prior art. As can be seen, the majority of the prior art only provides single figure percentage quit rates. Our method provides 20-40%, an enormous increase given the known difficulty associated with quitting smoking, and given the numerous prior art therapies which exist, none of which are nearly as successful as the present method.

It is also a substantial improvement on intensive behavioural support in combination with NRT or Zyban, which is particularly surprising, given the amount of time and personal effort that goes into this type of treatment. A benefit of the present method is that it is quick and easy and does not necessarily involve a lot of time and effort from the patient, such as the need to attend numerous counselling sessions, as is the case with the prior art.

A particularly preferred method of assessing the genetic information is described in GB 2338 301A (Zeneca Limited) hereby incorporated by reference.

Online Cognitive Behavioural Therapy (CBT)

Web Assisted Tobacco Intervention has been shown to be highly effective in helping smokers get mentally and physically prepared for their quit attempt. The proven interactive online process helps reinforce the changes that smokers have made to their lifestyle. The program has been validated by North American quitters since 1999. Both the American Cancer Society and the Canadian Cancer Society endorse and utilise this approach.

In other embodiments, CBT could be replaced or supplemented by managed care or professional advice.

The Nicotest™ online programme delivers a convenient and wide-reaching ‘managed care’ service for smokers. The Nicotest™ programme builds on the established NHS (National Health Service, UK) Smoking Cessation Clinics, by offering an effective, convenient low-cost, high-reach service for smoking cessation.

The Nicotest™ programme is currently undergoing a Phase IV style study to monitor progress in real-time. On the basis of a review of our data by eminent academics in this field, the present invention provides the following benefits:

1. Increase Quit Rates by Using a Structured Smoking Cessation Programme

Structured smoking cessation programmes have been shown to improve success rates.
   There is extensive evidence that behavioural treatment improves global long-term quit rates. The most widely studied behavioural intervention is adjunctive advice and instruction from a health care professional. Other programmes follow the principles of behaviour modification more closely, such as operant conditioning. Cinciripini et al., (1994) report that scheduled smoking (which removes the association between smoking urges and reinforcement) is a useful addition to multi-component treatment programmes. Cognitive-behavioural programmes to manage mood and withdrawal symptoms have also been shown to be effective
   References: Cornuz et al., 1997; Lifrak et al., 1997; Hall et al., 1994; Cinciripini et al., 1996; Cinciripini et al., 1994; Marlow and Stoller, 2003; Fortmann and Killen, 1995; Wright et al., 1996; Glasgow et al., 2000
      2. Increase Quit Rates by Taking the Right Medication at the Right Dose
Stratification by genotype identifies individuals who will respond better to nicotine replacement therapy.
   To date, five publications and two unpublished studies exist of gene-treatment interactions for smoking cessation pharmacotherapy.
   References: Learman et al., 2003; Swan et al., in press; Cinciripini et al., 2004; David et al., 2004; Munafo et al., 2004;
Stratification by genotype enables rate of metabolism of nicotine to be estimated.
   Genes that influence metabolic enzymes responsible for the metabolism of nicotine to cotinine have been extensively researched in relation to smoking behaviour, and there is evidence that they predict, for example, levels of cigarette consumption. There is good evidence for a functional effect of CYP2A6 genotype on nicotine metabolism (e.g. Zhang et al., 2002).
   Pianezza et al. (1998) first reported a lower incidence of CYP2A6 null alleles among tobacco dependent individuals compared to non-dependent individuals.
   Despite subsequent conflicting results (reviewed in Oscarson, 2001), a CYP2A6 genotype remains a strong candidate gene for smoking behaviour. Several studies have reported an association with smoking behaviour (typically level of dependence or cigarette consumption) (e.g. Tyndale et al., 1999; Rao et al., 2000; Iwahashi et al., 2004), although there have also been negative reports (e.g. Ando et al., 2003). The evidence from a recent meta-analysis (Munafò et al., 2004) indicated that CYP2A6 genotype may be associated with likelihood of being a former smoker. Inhibition of CYP2A6 activity in vivo has also been shown to alter smoking behaviour (e.g. Tyndale & Sellers, 2001; Sellers et al., 2003).
   Taken together, this evidence suggests that CYP2A6 genotype is likely to be associated with nicotine metabolism and, consequently, smoking behaviour (most probably consumption), but that this association may vary across ethnic groups in whom the prevalence of null alleles varies considerably.
   References: Zhang et al., 2002; Pianezza et al., 1998; Tyndale et al., 1999; Rao et al., 2000; Iwashi et al., 2004; Ando et al., 2003; Munafo et al., 2004; Tyndale and Sellers, 2001; Sellers et al., 2003.
The FTND allows a person's degree of dependence on nicotine to be estimated.
   The six-item Fagerström Test for Nicotine Dependence (FTND) is a reliable and valid self-reported measure of nicotine dependence (Fagerström et al., 1990). This is the most widely used measure of nicotine dependence (Benowitz, 1999), and has been reported to predict likelihood of smoking cessation success (Balfour et al., 2000), although only two items strongly predict likelihood of cessation (numbers of cigarettes per day, and time to first cigarette smoked).
   The FTND has also been used to identify sub-types of dependent smokers, and a cut-off score of 5 generally is recognized to indicate dependence (Lesch et al., 2004). Other measures of nicotine dependence, such as DSM-IV criteria, may underestimate the prevalence of nicotine addiction in the population (Benowitz, 1999).
   References: Fagerström et al., 1990; Benowitz, 1999; Balfour et al., 2000; Etter et al., 1999; Lesch et al., 2004
High-dose therapy of NRT improves quit rates for heavier smokers.
   High dose nicotine replacement therapy may improve success rates in heavy or highly dependent smokers. In addition to this tailoring nicotine replacement therapy dose based on cigarette consumption or FTND score enables lighter smokers to minimize the cost associated with nicotine replacement therapy.
   References: Gorsline et al., 1991; Bannon et al., 1989; Hughes et al., 1990; Dale et al., 1995; Tonnesen et al., 1999; Jorenby et al., 1995; Fredrickson et al., 1995; Benowitz et al., 1998; Hurt et al., 1993
      3. Increase Quit Rates by Using Professional Support During the Quit
Professional support during a smoking cessation attempt has been shown to improve success rates.
   Increasing the intensity of professional intervention has been widely reported to improve smoking cessation rates. The usual means by which the level of intervention or support is increased is by telephone contact following the initial intervention.
   References: Miller et al., 1997; Alterman et al., 2001; Reid et al., 1999.
      4. Increase Quit Rates by Combining Medication and Online Support
Nicotine replacement therapy has been demonstrated to be a safe and effective aid to smoking cessation.
Online support and medication increased the success rate from 4.5% for ‘cold turkey’ up to 18% for a randomised sample using a combined approach.
   References: West, R., McNeil, A. (2000)
      5. Increase Quit Rates with Personalised Treatment
Personalised treatment based on a person's readiness to stop smoking has been shown to improve success rates.
   References: Mogielnicki et al., (1986); Owens et al., (1989); Rimer and Orleans, 1994; Orleans, 2000; Secker-Walker et al., 1994; Bane et al., 1999; Strecher et al., 2000; Disjkstra and De Vriew, 1999; Borland et al., 2004; Mermelstein et al., 2003.
   The programme is refined and improved by on-going collection of data based on age, sex, lifestyle and ethnicity of the individuals. Thus, there is a feedback system, as discussed elsewhere leading to continuous improvement in the programme. This is a particular benefit of the present invention.
      Background Information on the Dopamine D2 Receptor and Smoking Cessation
      1) Role of Dopamine in Tobacco Addiction

Much of the pleasure derived from smoking tobacco is thought to come from activation of the dopamine system in the brain (Noble et al., 1994). The exact mechanism by which dopamine exerts its effects is not clear—it may be that dopamine release in the nucleus accumbens results directly in the perception of reward or that dopamine is involved in developing conditioned responses to environmental cues linked to the rewarding stimuli. Possible biological mechanisms are reviewed in Munafo et al., (2001).

2) Genetic Variation in DRD2

35% of people have a variant version of the gene that codes for the dopamine D2 receptor, which is the receptor thought to be important in the reward pathway.
Several studies have shown that the variant (DRD2 Taq1A) is linked to reduced dopamine binding in the brain (Jonsson et al., 1999; Thompson et al., 1997).
It is thought that dopamine lack causes a ‘reward deficit’ where people compensate for the deficiency using other substances such as alcohol and nicotine (Blum et al., 1996).
There is recently published evidence that the gene variant is related to personality traits and linked to level of alcohol consumption in moderate drinkers (Munafo et al., 2004).
Recent work shows that the DRD2 variant lies in a previously unknown gene encoding a protein kinase. The variant changes the amino acid composition of the protein encoded by the gene (Neville et al., 2004). The mechanism by which this change leads to effects on dopamine receptor density and substance use is, however, uncertain.
Several large and well-designed studies have shown that the variant form of the gene is more common in people who regularly smoke tobacco, for example (Comings et al., 1996).
There are, as one would expect, some studies that do not show this association—the scientific evidence is reviewed in (Munafo et al., 2001).
There is also evidence that current smokers who have the gene variant tend to consume more cigarettes, although the effect, if any, is likely to be small (McKinney et al., 2000; Johnstone et al., 2004).
   3) Genetic Effects on Smoking Cessation
One study showed that nicotine replacement is slightly more effective in the short term than placebo in people with the gene variant compared to people who have the common version of the gene (Johnstone et al., 2004).
Other studies suggest that people with the normal form of the gene respond well to bupropion, which is the alternative therapy commonly used for smoking cessation (Lerman et al., 2003; Swan et al., 2004).

Background to Online Support (for Instance Flight to Freedom Milestone 1 to 6)

Cognitive Behavioural Therapy (CBT) and Online Managed Care provides a high-reach low-cost solution for providing managed care at a primary level. “We find that managed care has made significant progress—and that opportunity remains”, (Cutler, 2003).

Farvolden (2003) found that web-based collaborative disease managements systems and behaviour modification have the potential to improve patient outcomes by providing increased support at the population level.

The benefits of the Internet, however, enable high-reach, low-cost personalised solution.

Three of the key features of the Internet and web-based management can be characterized as follows:
1) Data and Tracking: the ability to save and retrieve patient data at all stages of the disease management cycle, i.e. diagnosis, treatment, progress monitoring
2) Content Customization: the ability to represent the treatment process in a comprehensive and customized graphic format, i.e. the use of web browsers, graphic representation of stepped models of care
3) Interaction: the ability for patients to communicate directly with their primary care physician and other patients who are progressing through the same methods of treatment, i.e. via email, moderated support groups, instant messaging technology, counselling sessions, etc.

### Personalised Treatment can Improve Success Rates

“Web-based Support Groups for smoking cessation offer high-reach and personalised support for smokers at low cost per patient” (Fournier, 2004). Computerised tailoring of smoking cessation materials and advice has been investigated (e.g. Strecher et al., 1994; Disjkstra & De Vries, 1999; Strecher, 1999), generally indicating positive effects among moderate to light smokers. Such tailoring is dependent on the conceptual framework adopted, and is typically predicated on the Transtheoretical Model of behaviour change, so that readiness to change is the central variable used to tailor smoking cessation messages (Dijkstra et al., 1999).

The Addressing Tobacco in Managed Care (ATMC) initiative is based on the understanding that managed healthcare plans—which have information systems, defined populations, and other infrastructure to improve patient care—are well suited to implement, evaluate, and institutionalize tobacco control interventions. The results of the 2000 ATMC survey suggest that health plans have demonstrated great progress in their efforts to address tobacco use as one of the leading causes of preventable death and illness.

Jean-Francios Etter found that new computer-tailored cessation programs were effective in increasing cessation rates, “because it can reach large numbers of smokers, this program can substantially contribute to disease prevention at a population level” (Etter, J., Perneger, T., 2001.

The Necessity for a Personalised Treatment Programme to Augment Smoking Cessation: a Brief Evidence-Based Appraisal

It has been estimated that approximately 50% of the initiation of tobacco dependence is genetically influenced whereas the maintenance of dependent smoking behaviour and the amount smoked have approximately 70% genetic contribution (Carmelli et al., 1992; Heath et al., 1999; Koopmans et al., 1999; Madden et al., 1999; True et al., 1997). Genetic variation that affects response to medication and/or susceptibility to addiction/smoking behaviour may be somewhat distinct. This summary focuses on pharmacogenetics; the response to pharmacotherapy due to genetic profile (gene-treatment interaction) although raises the possibility for screening approaches to determine susceptibility to smoking behaviour/addiction.

Optimising Response to smoking Cessation Medication:

Research findings demonstrate the need for a treatment recommendation that is tailored to the individual in order to enable optimal treatment outcome: Therefore, a high-dose therapy of NRT improves quit rates for heavy or highly dependent smokers

It has been estimated that approximately 51% of the population are currently under-doses, and 11% are currently over-doses with nicotine replacement therapy (Johnstone et al., 2004). The currently recommended doses of nicotine replacement therapy (NRT) are inadequate for many smokers because NRT provides lower nicotine delivery in comparison to that delivered by smoking freely (reviewed in (Benowitz et al., 1998)). There is evidence that elevating the dose of NRT enhances the likelihood of successful cessation in more dependent smokers (reviewing in (Benowitz et al., 1998)) and there is also evidence that matching levels of NRT with those obtained from smoking freely enhances smoking cessation outcome (Saches, 1995). Hence, it seems likely that adjustment of NRT dosage according to individual parameters (‘personalised therapy’) could augment smoking cessation. This could be achieved using a pre-treatment biochemical estimate of nicotine and cotinine levels. An alternative method, which is likely to be more cost-effective, is to test smokers' genetic profiles to determine their individual control of nicotine metabolism. Such an approach would not only identify those with the need for high-dose therapy but would also enable lighter smokers to minimize the cost associated with NRT.

Therefore, an individual's rate of metabolism of nicotine impacts upon the optimal dose of NRT: stratification by genotype enables the rate of metabolism of nicotine to be estimated

1. CYP2A6

The extent of nicotine metabolism to its inactive metabolite, cotinine, must also be taken into account in order to make a recommendation of optimal therapeutic dose and minimise withdrawal symptoms (craving nicotine, increased irritability, depression or anxiety, increased appetite, difficulty concentrating, diminished attention span). In the majority of people, nicotine is extensively (70% to 80%) metabolized to cotinine by C-oxidation (Benowitz et al., 1994). Deficient C-oxidation of nicotine is associated with a longer half-life of nicotine and deficient generation of cotinine (Benowitz et al., 1995). Indeed, such deficient C-oxidation has been found to be associated with carriage of particular genetic variation within the gene encoding the CYP2A6 enzyme and such genetic variation has been found to influence the risks and addictiveness of tobacco use in affected individuals (as detailed below). This is perhaps not surprising considering that the human cytochrome (P450 2A6 (CYP2A6) enzyme is responsible for the majority of the metabolic inactivation of nicotine to its inactive metabolite cotinine (Benowitz and Jacob, 1994; Messina et al., 1997; Nakajima et al., 1996; Zhang et al., 2002). CYP2A6 also contributes to the N-demethylation of nicotine to nornicotine (Yamanaka et al., 2005).

In recent years, 14 genetic variants CYP2A6*1-*2-*12 and the gene duplication, *1×2) of CYP2A6 have been identified and a number of these have been shown to result in altered CYP2A6 enzyme activity. For example, there are alleles which result in variants that are in inactive (e.g. due to a gene deletion), have decreased activity (e.g. altered enzyme structure or transcriptional activity) or have increased activity (e.g. due to gene duplications). The resulting interindividual variation in metabolic activity can affect the metabolism of nicotine (reviewed by (Xu et al., 2002)) (further details and information on haplotype studies of this gene: on request). Both in vitro and in vivo studies have demonstrated considerable inter-individual variation in CYP2A6 activity (Iscan et al., 1994; Rautio et al., 1992; Yamano et al., 1990).

Regarding the underlying genetics, individuals carrying one or two null (inactive) CYP2A6 alleles (‘slow metabolisers’) have impaired nicotine metabolism (Messina et al., 1997) resulting in an prolonged nicotine exposure in the bloodstream. These individuals have been found to be significantly protected against becoming tobacco-dependent smokers. In addition, smokers whose nicotine metabolism is impaired smoke significantly fewer cigarettes than those with normal nicotine metabolism (Iwahashi et al., 2004; Messina et al., 1997; Minematsu, 2006; Pianezza et al., 1998; Rao et al., 2000; Sellers et al., 2003; Tyndale et al., 1999; Tyndale and Sellers 2001): this could be useful as a screening tool as well as a prognostic test of likely dose of NRT required. Reciprocally, a duplication variant in the gene encoding CYP2A6 has been identified which increases nicotine inactivation and increased smoking (Rao et al., 2000). CYP2A6 can also activate tobacco smoke procarcinogens and carriage of CYP2A6-null alleles genetic variation in the gene encoding CYP2A6 has been linked with susceptibility to lung and oesophageal cancers (Ariyoshi et al., 2002; Fujieda et al., 2004; Miyamoto et al., 1999; Tan et al., 2001). CYP2A6 has been shown to have large interethnic variability in levels of expression and activity. This is thought to be largely due to differences in allele frequencies (for example, a higher frequency of CYP2A6 Gene deletion in Asians compared to European populations (references on request).

In summary, despite some conflicting results (reviewed by (Oscarson, 2001)), the CYP2A6 genotype remains of particular importance for an individual's need for nicotine and may significantly affect nicotine levels from sources other than cigarettes, e.g. nicotine-replacement therapies, of for long-term maintenance against tobacco dependence.

Dopamine beta-hydroxylase (DBH) catalyzes the oxidative hydroxylation of dopamine to noradrenaline (Kaufman and Friedman, 1965). DBH enzyme levels have been suggested to modulate both dysphoric and rewarding effects of pyschostimulants in humans (Cubells et al., 2000). Plasma DBH activity varies widely between individuals. Allelic variations in the DBH and monoamine oxidase genes have 3 been found to be predictive as to whether a person is a heavy smoker and how many cigarettes they consume; heavier smokers carried the DBH 1368A allele when compared to light smokers; conversely, heavier smokers were less likely to harbour the MASOA 1460C allele (McKinney et al., 2000).

Hence, as was the case of CYP2A6, these results support the view that these enzymes help to determine a smoker's requirement for nicotine and help to explain why some people are predisposed to tobacco addiction and why some find it particularly difficult to stop smoking. This offers potential for developing patient-specific therapy for smoking cessation as well as having important implications for smoking prevention.

Therefore, An individual's genetic profile can predict response to a particular treatment; stratification by genotype and gender identifies individuals who will respond better to nicotine replacement therapy or to bupropion

Pharmacological intervention for smoking cessation includes several forms of NRT and the use of sustained-release bupropion (amfebutamone; Zyban™). Although both treatments have demonstrable efficacy relative to placebo, substantial inter-individual variability exists in terms of therapeutic response and relapse after such intervention occurs in 70-80% of patients within 12 months (Dale et al., 2001; Diefenbacher et al., 2003; Ferry and Johnston, 2003; Fiore et al., 1994; Hurt et al., 1997; Rodriguez-Artalejo et al., 2003)>

The most commonly noted side-effects associated with bupropion (Ferry and Johnston, 2003; Fiore, 2000), often presenting throughout the course of treatment, are insomnia (estimated to affect 35-45% of bupropion users) and dry mouth (estimated to affect 35-45% of bupropion due to side-effects alone (Fiore, 2000).

2. DRD2 TAQ1A

Pleasure derived from smoking tobacco is thought to arise as a result of activation of the dopamine system, and in particular the dopamine D2 receptor (DRD2) in the brain (Noble et al., 1994). Although the exact mechanism by which dopamine exerts its effects is unclear, the body of literature suggests that a lack of dopamine causes a ‘reward deficit’ whereby people compensate for the deficiency through the use of other substances such as alcohol and nicotine (reviewed by (Blum et al., 1996)). DRD2 is centrally involved in reward-mediating mesocorticolimbic pathways and, as such, ahs been the focus of many studies investigating genetic variation associated with addictive behaviour.

1. A Higher Prevlence of Carriage of the DRD2 TAQ1A Variant in Smokers

One such polymorphic locus linked to DRD2 gene, designated Taq1A, has been implicated in smoking behaviour and alcoholism (reviewed by (Comings and Blu, 2000)) and functional studies have indicated that the DRD2 Taq1A variant is linked to reduced DRD2 receptor density in the brain (Jonsson et al., 1999; Pohjalainen et al., 1998); Thompson et al., 1997), although this is not universally accepted (Laruelle et al., 1998). Lower D2 receptor affinity have also been reported in women compared to men, suggesting an increased endogenous striatal dopamine concentration in women (Pohjalainen et al., 1998). This may have implications for the differential vulnerability of men and women to substance dependence. Several large and well-designed studies, including meta-analyses, have reported a significantly higher prevalence of the Taq1A allele in regular smokers than in non-smokers (Comings et al., 1996; Li et al., 2004; Munafo et al., 2004) and there is evidence that smokers who harbour this gene variant tend to consume more cigarettes (McKinney et al., 2000). The DRD2 genotype does not, however, appear to predict susceptibility to taking up smoking (Berlin et al., 2005) although the authors acknowledge that it may be valuable in the prediction of response to medication. Recent work shows that the DRD2 Taq1A variant lies in a previously unknown gene (ANKK1) encoding a protein kinase. The variant changes the amino acid composition of the protein that the gene encodes and may affect substrate-binding specificity (Neville et al., 2004).

2. Women are More Likely to Respond Well to NRT if They Carry the DRD2 TAQ1A Allele Wherease Carriers of the Common Version of the Allele Respond Well to Bupropion

A recent Cancer Research UK-funded large randomised controlled trial has reported that the effectiveness of nicotine patches was higher in women with the Taq1A variant compared to those with the more common genotype. No difference was observed in men, suggesting that the determination of the DRD2 polymorphisms in interaction with NRT and gender may predict a smoking cessation outcome (Johnstone et al., 2004; Yudkin et al., 2004). It appears that people with the common version of the gene (Potentially due to gene-gene interaction) respond well to bupropion, which is the alternative therapy commonly used for smoking cessation (Lerman et al., 2003; Swan et al., 2005).

3. Women are Likely to Suffer Side-Effects to Pubropion if They Carry DRD2 TAQ1A allele

Bupripion's efficacy in the treatment of nicotine dependence, possibly due to its inhibition of dopamine and noradrenaline uptake and/or its nicotinic receptor antagonism, may be particularly effective in smokers who are more prone to relapse, such as African Americans (Ahluwalia et al., 2002)) and smokers with higher daily smoking rates (Dale et al., 2001). However, as well as the finding that women carrying the Taq1A variant respond well to NRT (Yudkin et al., 2004), it has also recently been reported that carriage of this allele in women (prevalence of carriage being an estimated 41% of the Caucasian population studied) pre-disposes to side-effects from bupropion; compared to women who carry both common alleles, women with at least one Taq1A allele were more likely to report having stopped taking bupropion due to medication side-effects and at 12 months were more likely to be still smoking (no significant trends were observed in men) (Swan et al., 2005). The allele frequency of the Taq1A polymorphism has been estimated to be 41% in Caucasian men and women, increasing in other ethnic groups such as those of Asian or African Indian descent.

A recent study on a small population of smokers has tentatively concluded that bupropion may be more beneficial for smokers homozygous for the DRD2-141 Ins C allele, while NRT may be more beneficial for smokers carrying the Del C allele although this would require confirmation in additional larger study samples before applying to clinical practice (Lerman et al., 2006). Interestingly, the Taq1′A1′ allele is in linkage disequilibrium with this polymorphism and other mutations of functional significance (Duan et al., 2003).

A further study has suggested that carriers of a particular genetic variant of CYP2B6 may be roe vulnerable to abstinence symptoms and relapse. Bupropion may attenuate these effects, especially among females (Lerman et al., 2002) (again, this suggestive finding would require confirmation in a larger study before being clinically applicable.

### REFERENCES

All of the references provided are hereby incorporate by reference, to the extent that there is no inconsistency with the present disclosure.

### REFERENCES FOR GENETIC TEST

Blum K, Sheridan P J, Wood R C, Braverman E R, Chen T J, Cull J G, Comings D E. The D2 dopamine receptor gene as a determinant of reward deficiency syndrome. J R Soc Med 1996; 89(7):396-400.

Comings D E, Ferry L, Bradshaw-Robinson S, Burchette R, Chiu C, Muhleman D. The dopamine D2 receptor (DRD2) gene: a genetic risk factor in smoking. Pharmacogenetics 1996; 6(1):73-9.

Jonsson E G, Nothen M M, Grunhage F, Farde L, Nakashima Y, Propping P, Sedvall G C. Polymorphisms in the dopamine D2 receptor gene and their relationships to striatal dopamine receptor density of healthy volunteers. Mol Psychiatry 1999; 4(3):290-6.

Johnstone E C, Yudkin P L, Hey K, Roberts S J, Welch S J, Murphy M F, Griffiths S E, Walton R T. Genetic variation in dopaminergic pathways and short-term effectiveness of the nicotine patch. Pharmacogenetics 2004; 14(2):83-90.

Lerman et al. (2002). Pharmacogenetic investigation of smoking cessation treatment. Pharmacogenetics, 12, 627-634.

Lerman C, Shields P G, Wileyto E P, Audrain J, Hawk L H, Jr., Pinto A, Kucharski S, Krishnan S, Niaura R, Epstein L H. Effects of dopamine transporter and receptor polymorphisms on smoking cessation in a bupropion clinical trial. Health Psychol 2003; 22(5):541-8.

Munafo M, Johnstone E, Murphy M, Walton R. New directions in the genetic mechanisms underlying nicotine addiction. Addict Biol 2001; 6(2):109-117.

Munafo M R, Clark T G, Moore L R, Payne E, Walton R, Flint J. Genetic polymorphisms and personality in healthy adults: a systematic review and meta-analysis. Mol Psychiatry 2003; 8(5):471-84.

Munafo' M, Johnstone E, Welsh K, Walton R. Association between the DRD2 gene TAq1A polymorphism and alcohol consumption in social drinkers (in press). The Pharmacogenomics Journal 2004.

Munafò et al. (2004). The genetic basis for smoking behavior: A systematic review and meta-analysis. Nicotine and Tobacco Research, 6, 583-597.

Neville M J, Johnstone E C, Walton R T. Identification and characterization of ANKK1: a novel kinase gene closely linked to DRD2 on chromosome band 11q23.1. Hum Mutat 2004; 23(6):540-5.

Noble E P, St. Jeor S T, Ritchie T, Syndulko K, St. Jeor S C, Fitch R J, Brunner R L, Sparkes R S. D2 dopamine receptor gene and cigarette smoking: a reward gene? Med Hypotheses 1994; 42(4):257-60.

McKinney E F, Walton R T, Yudkin P, Fuller A, Haldar N A, Mant D, Murphy M, Welsh K I, Marshall S E. Association between polymorphisms in dopamine metabolic enzymes and tobacco consumption in smokers. Pharmacogenetics 2000; 10(6):483-91.

Swan G E, Valdes A M, Ring H Z, Khroyan T V, Jack L M, Ton C C, Curry S J, McAfee T. Dopamine receptor DRD2 genotype and smoking cessation outcome following treatment with bupropion SR. Pharmacogenomics J 2004.

Thompson J, Thomas N, Singleton A, Piggott M, Lloyd S, Perry E K, Morris C M, Perry R H, Ferrier I N, Court J A. D2 dopamine receptor gene (DRD2) Taq1 A polymorphism: reduced dopamine D2 receptor binding in the human striatum associated with the A1 allele. Pharmacogenetics 1997; 7(6):479-84.

Yudkin P, Munafo M, Hey K, Roberts S, Welch S, Johnstone E, Murphy M, Griffiths S, Walton R. Effectiveness of nicotine patches in relation to genotype in women versus men: randomised controlled trial. BMJ 2004; 328(7446):989-90.

### REFERENCES FOR CBY (COGNITIVE BEHAVIOURAL THERAPY)

Alterman A I et al. (2001). Short- and long-term smoking cessation for three levels of intensity of behavioral treatment. Psychology of Addictive Behaviours, 15, 261-264.

Bane C M et al. (1999). Development of a pregnancy-tailored decisional balance measure for smoking cessation. Addictive Behaviors, 24, 795-799.

Bannon Y B et al. (1989). Transdermal delivery of nicotine in normal human volunteers: a single dose and multiple dose study. European Journal of Clinical Pharmacology, 37, 285-290.

Benowitz N L et al. (1998). Suppression of nicotine intake during ad libitum smoking by high-dose transdermal nicotine. Journal of Pharmacology and Experimental Therapeutics, 287, 958-962.

Cinciripini P M et al. (1994). An evaluation of a multicomponent treatment program involving scheduled smoking and relapse prevention procedures: initial findings. Addictive Behaviors, 19, 13-22.

Cinciripini P M et al. (1996). Behavior therapy and the transdermal nicotine patch: effects on cessation outcome, affect, and coping. Journal of Consulting and Clinical Psychology, 64, 314-323.

Cornuz J et al. (1997). Smoking cessation counseling by residents in an outpatient clinic. Preventive Medicine, 26, 292-296.

Cutler, C., Preventative medicine in managed care, Jun. 25, 2002, VOL. 3, NO. 3

Dale L C et al. (1995). High-dose nicotine patch therapy: percentage of replacement and smoking cessation. Journal of the American Medical Association, 274, 1353-1358.

Dijkstra A & De Vries H (1999). The development of computer-generated tailored interventions. Patient Education and Counseling, 36, 193-203.

Dijkstra A et al. (1999). Targeting smokers with low readiness to change with tailored and nontailored self-help materials. Preventive Medicine, 28, 203-211.

Etter J F & Pemeger T V (2001). Effectiveness of a computer-tailored smoking cessation program: a randomized trial. Archives of Internal Medicine, 161, 2596-2601.

Farvolden, P and Mierlo, T, (2003). Web-based collaborative disease management and health behaviour change [Available at http://www.v-cc.com/research/]

Fortmann S P & Killen J D (1995). Nicotine gum and self-help behavioral treatment for smoking relapse prevention: results from a trial using population-based recruitment. Journal of Consulting and Clinical Psychology, 63, 460-468.

Fournier, R., (2004). NRT and Buproprian usage amoung users of a web-based smoking cessation support group [Available from: http://www.v-cc.com/research/]

Fredrickson P A et al. (1995). High dose transdermal nicotine therapy for heavy smokers: safety, tolerability and measurement of nicotine and cotinine levels. Psychopharmacology, 122, 215-222.

Gorsline J et al. (1991). Nicotine dose relationship for Nicoderm at steady state. Pharmaceutical Research, 10, S299.

Hall S M et al. (1994). Cognitive-behavioral intervention increases abstinence rates for depressive-history smokers. Journal of Consulting and Clinical Psychology, 62, 141-146.

Hughes J R et al. (1990). Effect of dose on nicotines reinforcing, withdrawal-suppression and self-reported effects. Journal of Pharmacology and Experimental Therapeutics, 252, 1175-1183.

Hurt R D et al. (1993). Serum nicotine and cotinine levels during patch therapy. Clinical Pharmacology and Therapeutics, 54, 98-106.

Jorenby D E et al. (1995). Varying nicotine patch dose and type of smoking cessation counseling. Journal of the American Medical Association, 274, 1347-1352.

Lifrak P et al. (1997). Results of two levels of adjunctive treatment used with the nicotine patch. American Journal of Addiction, 6, 93-98.

Mermelstein R et al. (2003). Extended telephone counseling for smoking cessation: does content matter? Journal of Consulting and Clinical Psychology, 71, 565-574.

Mogielnicki R P et al. (1986). Tailored media can enhance the success of smoking cessation clinics. Journal of Behavioral Medicine, 9, 141-161.

Marlow S P & Stoller J K (2003). Smoking cessation. Respiratory Care, 48, 1238-1254.

Miller N H et al. (1997). Smoking cessation in hospitalized patients. Results of a randomized trial. Archives of Internal Medicine, 157, 409-415.

Orleans C T & Fishman J (2000). Tailored communications for smoking cessation. Introduction. Tobacco Control, 9, 149.

Orleans C T et al. (2000). Computer tailored intervention for older smokers using transdermal nicotine. Tobacco Control. 9, 153.

Owens N et al. (1989). Smoking cessation by mail: a comparison of standard and personalized correspondence course formats. Addictive Behaviors, 14, 355-363.

Rimer B K & Orleans C T (1994). Tailoring smoking cessation for older adults. Cancer, 74, 2051-2054.

Reid R D et al. (1999). Is telephone counselling a useful addition to physician advice and nicotine replacement therapy in helping patients to stop smoking? A randomized controlled trial. Canadian Medical Association Journal, 160, 1577-1581.

Secker-Walker R H et al. (1994). Individualized smoking cessation counseling during prenatal and early postnatal care. American Journal of Obstetrics and Gynecology, 171, 1347-1355.

Strecher V J (1999). Computer-tailored smoking cessation materials: a review and discussion. Patient Education and Counseling, 36, 107-117.

Tonnesen P et al. (1999). Higher dosage nicotine patches increase one-year smoking cessation rates: results from the European CEASE study. European Respiratory Journal, 13, 238-246.

Wright L N (1996). Statewide assessment of a behavioral intervention to reduce cigarette smoking by pregnant women. American Journal of Obstetrics and Gynecology, 175, 283-287.

### REFERENCES FOR DISCUSSION OF SMOKING-RELATED GENES

Audrain J, Rodriguez D. et al (2004) Identifying and Characterizing Adolescent Smoking Trajectories. Working paper

Austin M A, K. M., Bawol R D, et al. (1987). “Risk factors for coronary heart disease in adult female twins: genetic heritability and shared environmental influences.” Am J Epidemiol 125: 308-318.

Bank, W. (1993). Devlopment Report: 1993. New York, Oxford University Press.

Becker, G. S. (1993). Human Capital: A Theoretical and Empirical Analysis with Special Reference to Education, The University of Chicago Press.

Behrman, J. R., A. D. Foster, et al. (1999). “Women's Schooling, Home Teaching, and Economic Growth.” Journal of Political Economy 107(4): 682-714.

Behrman, J. R., M. R. Rosenzweig, et al. (1994). “Endowments and the Allocation of Schooling in the Family and in the Marriage Market: The Twins Experiment.” Journal of Political Economy 102(6): 1131-1174.

Behrman, J. R., M. R. Rosenzweig, et al. (1996). “College Choice and Wages: Estimates Using Data on Female Twins.” The Review of Economics and Statistics 78(4): 672-685.

Boustead C, T. H., Idle J R, et al. (1997). “CYP2D6 genotype and smoking behaviour in cigarette smokers.” Pharmacogenetics 7(411-414).

Carmelli D, S. G., Robinette D, et al. (1990). “Heritability of substance use in the NAS-NRC Twin Registry.” Acta Genet Med Gemellol 39: 91-98.

Currie, J. (1998). Child Health in Developed Countries.

Currie, J. and E. Moretti (2000). Mother's Education and the Intergenerational Transmission of Human Capital: Evidence from College Openings.

Douglas S, H. G. (1994). “The hazard of starting smoking: estimates from a split population duration model.” Journal of Health Economics 13(2): 213-230.

Farrelly M C, B. J. (1998). “Response to increases in cigaretteprices by race/ethnicity, income, and age groups.” Morbidity and Mortality Weekly Report 47(29): 605-609.

Fisher (1958). “Lung cancer and cigarettes [letter].” Nature 182: 108.

FUCHS, V. (1982). Time Preference and Health: An Exploratory Study, University of Chicago Press: 93-120.

Gruber Jonathan (2001). “Youth Smoking in the 1990s: Why Did it Rise and What are the Long Run Implications?” American Economic Review 91(2): 85-90.

Ham, J., J. Kagel and S. Lehrer (2002). “Randomization, Endogeneity and Laboratory Experiments in Economics.” Journal of Econometrics(Forthcoming).

Heckman, J. J. (1978). “A Partial Survey of Recent Research on the Labor Supply of Women.” American Economic Review 68(2): 200-207.

Jorm A F, H. A., Jacomb P A, et al. (2000). “Association of smoking and personality with a polymorphism of the dopamine transporter gene: results from a community survey.” Am J Med Genet 96: 331-334.

Keeler T E, H. T.-W., Barnett P G, Manning (1993). “Taxation, regulation and addiction: a demand function for cigarettes based on time-series evidence.” Journal of Health Economics 12(1): 1-18.

Kimihide Yoshida, N. H., Ken-ichi Kozaki, Hiroko Saito, Ken Maeno, Takahiko Sugiura, Katashi Ookuma and Takashi Takahashi (2001). “Association between the Dopamine D2 Receptor A2/A2 Genotype and Smoking Behavior in the Japanese.” Cancer Epidemiology Biomarkers & Prevention 10: 403-405.

Leone, B. V. P. A. B. W. W. S. (2003). “The genetic determinants of smoking. [comment]. [Review].” Chest 123(5): 1730-1739.

Lerman, C., et al (2001). “Tryptophan hydroxylase gene variant and smoking behavior.” American Journal of Medical Genetics 105(6): 518-520.

Lerman C, C. N., Audrain J, et al. (1999). “Evidence suggesting the role of specific genetic factors in cigarette smoking.” Health Psychology 18: 14-20.

Mayhew K P, F. B., Mott J A (2000). “Stages in the development of adolescent smoking.” Drug and Alcohol Dependence 59(S1): S61-S81.

Meltzer D. (1992). “Mortality Decline, Human Capital Investment and Economic Growth.” Dissertation.

Noble E P, S. J. S., Ritchie T, et al. (1999). “ND2 dopamine receptor gene and cigarette smoking: a reward gene?” Med Hypotheses 42(257-260).

WHO (1997). Tobacco or Health: A Global Status Report. Geneva, World Health Organization.

Pianezza M L, S. E. T. R. (1998). “Nicotine metabolism defect reduces smoking.” Nature 393: 750.

Rabin M, O. D. E. (2001). ““Choice and Procrastination”.” Quarterly Journal of Economics: 121-160.

Rosenzweig, M. R. (1995). “Why Are There Returns to Schooling?” The American Economic Review 85(2, Papers and Proceedings of the Hundredth and Seventh Annual Meeting of the American Economic Association Washington, D.C., Jan. 6-8, 1995): 153-158.

Rosenzweig, M. R. and K. I. Wolpin (1994). “Are There Increasing Returns to the Intergenerational Production of Human Capital? Maternal Schooling and Child Intellectual Achievement.” Journal of Human Resources 29(2, Special Issue: Women's Work, Wages, and Well-Being): 670-693.

Rosenzweig, M. R. and K. I. Wolpin (2000). “Natural “Natural Experiments” in Economics.” Journal of Economic Literature 38(4): 827-874.

Sander, W. (1995). “Schooling and Quitting Smoking.” The Review of Economics and Statistics 77(1): 191-199.

Schoemaker, P. (1982). “The Expected Utility Model: Its Variants, Purposes, Evidence and Limitations.” JEL 20(2): 529-563.

Singleton A. B., T. J. H., Morris C. M., Court J. A., Lloyd S., Cholerton S. (1998). “Lack of association between the dopamine D2 receptor gene allele DRD2*A1 and cigarette smoking in a United Kingdom population.” Pharmacogenetics 8(125-128).

Sullivan, P. F., et al. (2001). “Association of the tryptophan hydroxylase gene with smoking initiation but not progression to nicotine dependence.” American Journal of Medical Genetics 105(5): 479-484.

Sullivan P F, K. K. (1999). “The genetic epidemiology of smoking.” Nicotine Tob Res 1: S51-S57.

Thompson J, T. N., Singleton A, Piggott M, Lloyd S, Perry E K, Morris C M, Perry R H, Ferrier I N, Court J A. (1997). “D2 dopamine receptor gene (DRD2) Taq1A polymorphism: reduced dopamine D2 receptor binding in the human striatum associated with the A1 allele.” Pharmacogenetics 7(6): 479-484.

Wacholder S, R. N., Caporaso N. (2000). “Population stratification in epidemiologic studies of common genetic variants and cancer: quantification of bias.” Journal of the Natl Cancer Inst 92(115).

Wacholder Sholom, N. R. and N. Caporaso (2002). “Counterpoint: Bias from Population Stratification Is Not a Major Threat to the Validity of Conclusions from Epidemiological Studies of Common Polymorphisms and Cancer.” Cancer Epidemiology, Biomarkers & Prevention 11: 513-520.

Walque, d. (2002). “How Does Education Affect Health Decisions? The Cases of Smoking and HIV/AIDS.” Working Paper.

Warner, C. F. (1998). “The Economics of Smoking.” The Handbook of Health Economics.

### EXAMPLE OF GENETIC INFORMATION ASSESSMENT

SAFEspot™

SAFEspot, available from DSX Genotyping, 48 Grafton Street, Manchester, UK, is a revolutionary blood sample collection and storage system based on a proprietary adsorbent matrix.

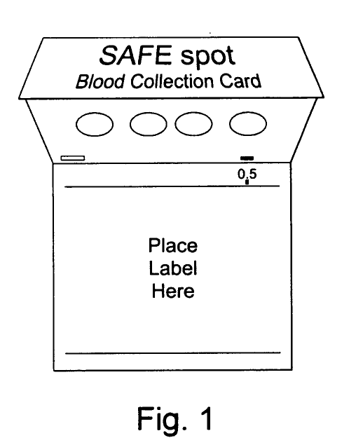

FIG. 1 shows a DxS SAFEspot™ device (a blood collection card). Four sample spots are shown where the blood is adsorbed onto the proprietary matrix

In the past, many genotyping studies have used liquid blood as the source of genetic material, today the same tests may be conducted from a single blood spot. The SAFEspot blood collection card is available to simplify the sample collection process.
Simplicity in blood collection and transport
A dried blood sample is safe, sterile and stable.
Ambient shipment, no dry ice requirement.
From each spot, sufficient DNA is captured for over 200 separate SNP, microsatellite or other genetic analyses.
Cards can be tailored for individual studies, with pre-printed labels.
A single finger stick blood sample, means that collection is painless and trauma free.
Tamper proof packaging ensures sample integrity.
Cards are compatible with in-house clinical data coding procedures.
Ideal for DNA archiving supported by 10 years stability data.

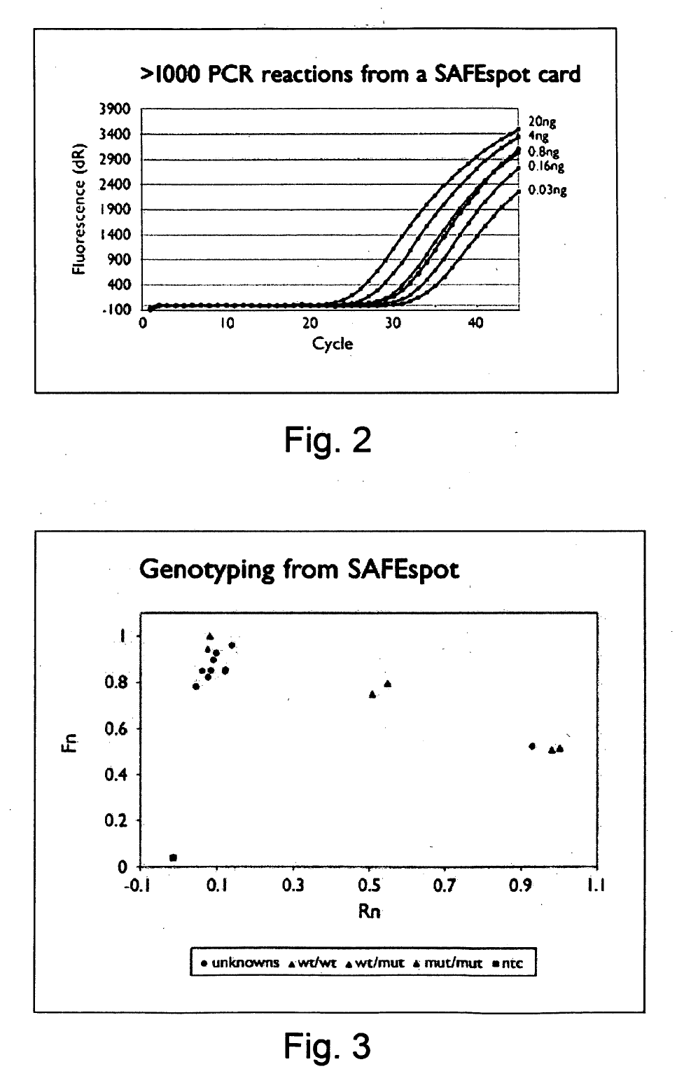

FIG. 2 is a graph of standard DNA and SAFEspot DNA. Standard DNA is shown by the first three traces from the left, and the first two traces from the right, when viewed at 900 dR. The SAFEspot DNA is the third trace from the right, when viewed at 900 dR. 0.5 μl of SAFEspot DNA is equivalent to 0.8 ng of standard DNA. The total volume of SAFEspot DNA per card is a least 800 μl, which is enough DNA for >1000 PCR reactions.

FIG. 3 shows that DNA from SAFEspot produces clean genotyping data for a typical Drug Metabolising Enzyme gene. Genotyping study carried out using Scorpions technology.

Use of Scorpions™ and ARMS™ Technology

Scorpions™ is a homogeneous or closed-tube platform for PCR analysis. It is ideal for all diagnostic applications which require rapid and reliable PCR detection such as viral load testing, RNA profiling, pharmacogenomics and environmental analysis. Scorpions is a class leading technology with significant technical benefits over competing technologies. Product development programmes utilising DxS' experience helps companies expedite the launch of next generation molecular diagnostics. Significantly, Scorpions is available for license from DxS Ltd.

The technology is described in more detail in GB2338301, U.S. Pat. No. 6,326,145 and U.S. Ser. No. 03/087,240, all of which are incorporated herein by reference.

Benefits of Using the Scorpions™ System:

High Sensitivity—the limit of detection is a few molecules even in the presence of very high levels of background DNA.
High Specificity—both the Scorpion primer region and probe region can be made sequence specific. This gives unparalleled discrimination allowing the detection of single nucleotide changes even in admixtures where the alternative sequence is in vast excess.
High Speed—Scorpions signal generation is widely recognised as the fastest in its class. This means that the technology can support very rapid PCR allowing detection in less than ten minutes.
Can function successfully even in areas of difficult primary and secondary structure: so-called “Difficult Sequences.” The intra-molecular signal generation mechanism means that Scorpions are particularly good at detecting targets in regions of high G+C content or secondary structure.
   The Scorpions Reaction

Scorpions are bi-functional molecules containing a PCR primer covalently linked to a probe. The fluorophore in the probe interacts with a quencher which reduces fluorescence. During a PCR reaction the fluorophore and quencher are separated which leads to an increase in light output from the reaction tube. There are two formats for Scorpions. The diagram below shows the bi-molecular Scorpion format. The alternative is known as the unimolecular format in which an integral stem loop sequence is used to bring the quencher close to the fluorophore.

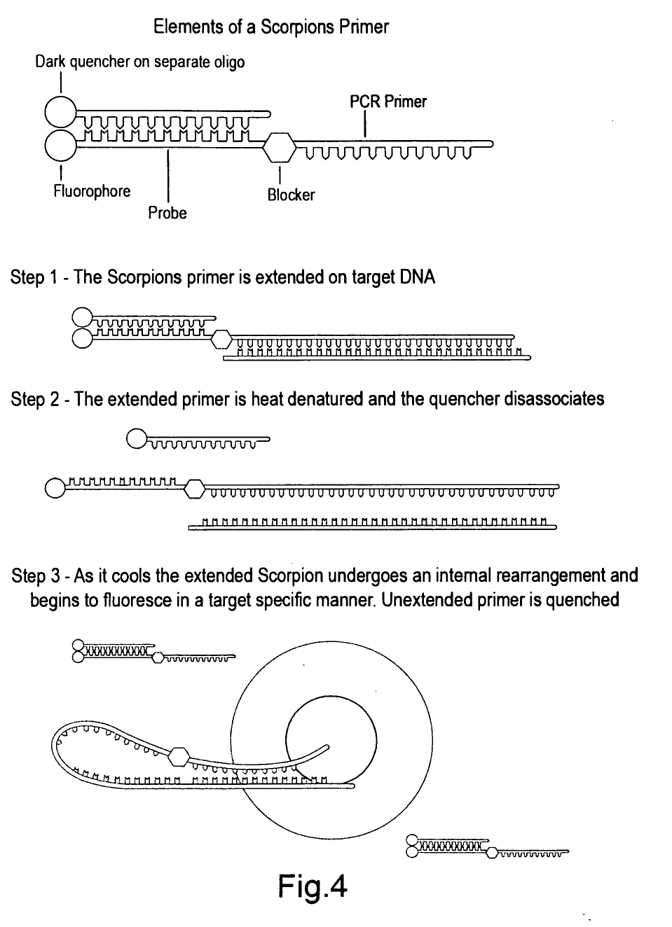

FIG. 4 is a schematic of the Scorpions system. Scorpions are also described more fully in Whitcombe, D., Theaker J., Guy, S. P., Brown, T., Little, S. (1999)—Detection of PCR products using self-probing amplicons and fluorescence. Nature Biotech 17, 804-807, which can be downloaded from the DxS website (www.dxsgenotvping.com/bibiography.htm)

Scorpions Performance Data

1) Real-Time PCR

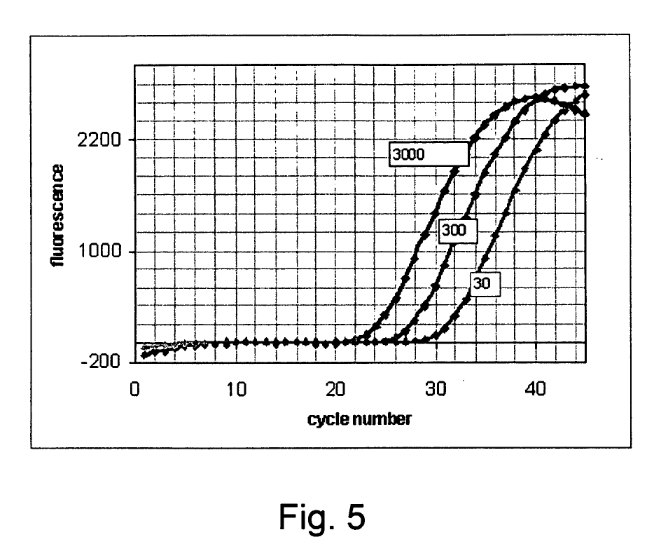

A Scorpion specific for a region of the human β-actin gene was used to amplify human genomic DNA. The Scorpions sequence and reaction conditions are available on request. FIG. 5 shows the data generated using either 3000, 300 or 30 copies of genomic:

FIG. 5 shows the typical smooth reaction profile associated with a Scorpions reaction. The data is also very reproducible; the standard deviations of the Ct (threshold cycle) values of the three dilutions were 0.27, 0.34 and 0.94 cycles.

2) Quantitative PCR

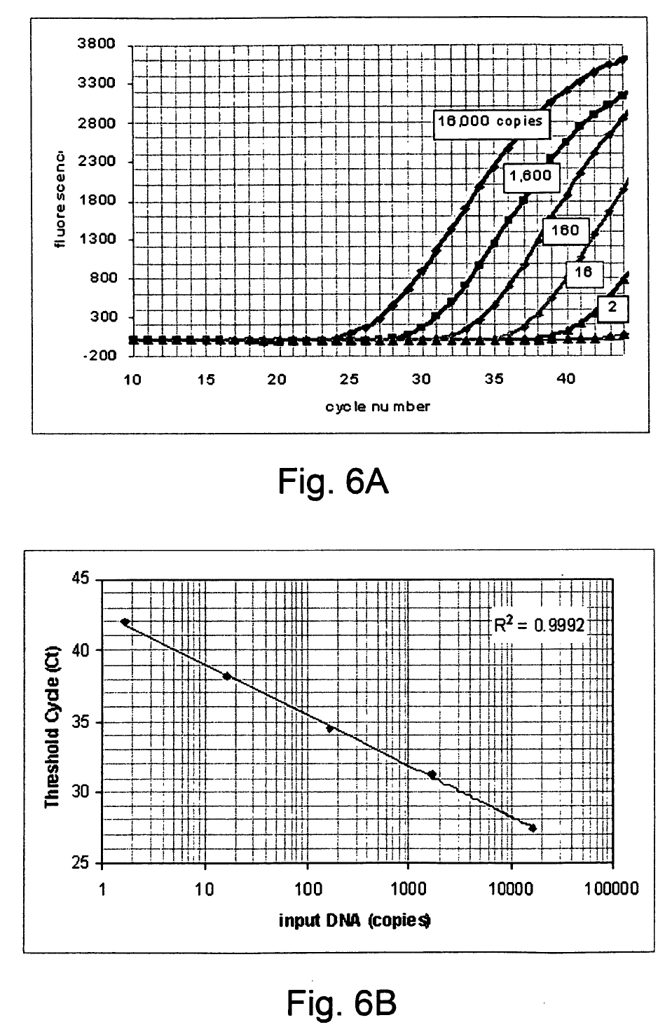

The same β-actin Scorpions reaction was also used to demonstrate quantitative nucleic acid detection down to the lowest possible level. FIG. 6A shows the data generated using dilutions of genomic DNA ranging from 16,000 to 1.6 copies per Scorpions reaction.

Re-plotting the data, as shown in FIG. 6B, shows the excellent logarithmic relationship between input DNA and threshold cycle.

3) One-Step RNA Detection

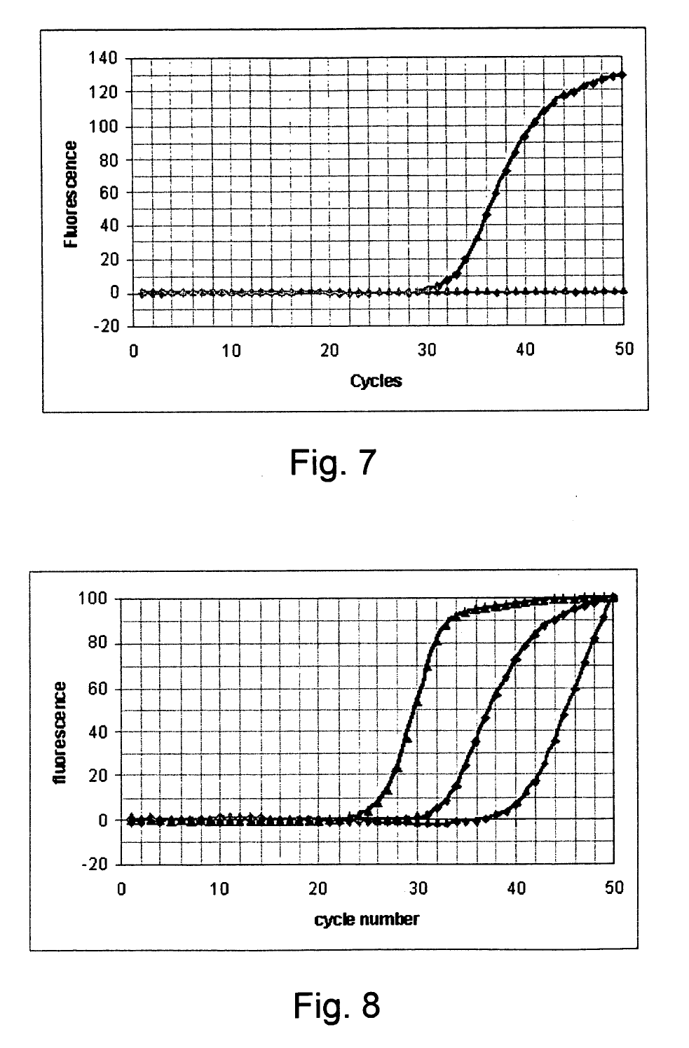

The Scorpions reaction is compatible with one-step reverse transcription PCR. The data shown in FIG. 7 shows the detection of an RNA transcript. The second lower trace was obtained using genomic DNA and shows that the Scorpions primer is only detecting PCR products derived from RNA.

4) Multiplex RNA Detection

Scorpions can also be multiplexed together and the graph shown in FIG. 8 shows multiplex detection of 3 separate RNA species in a single reaction:

FIG. 8 shows three traces from three separate Scorpions specific to three different RNA transcripts. Three separate dyes were used to allow differential detection of the three species

5) Rapid Detection

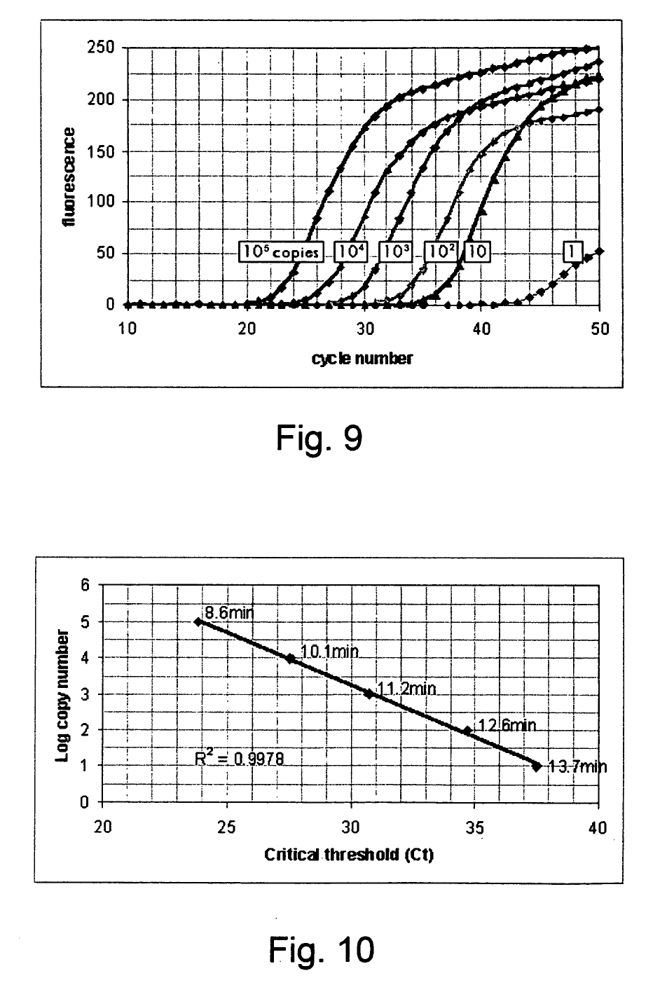

For some applications, very rapid detection of the target nucleic acid is important. For example, the detection of bio-threat agents or for point-of-care diagnostics. The unique unimolecular nature of the Scorpions reaction means that it is ideally suited for fast PCR analysis. The data shown in FIG. 9 was generated using a Cepheid SmartCycler instrument. Scorpions primers were used to rapidly detect low levels of Bacillus spp.

The higher levels of input DNA were detected in less than 10 minutes and even very low levels could be detected in under 14 minutes, as shown in FIG. 10.

6) Genotyping

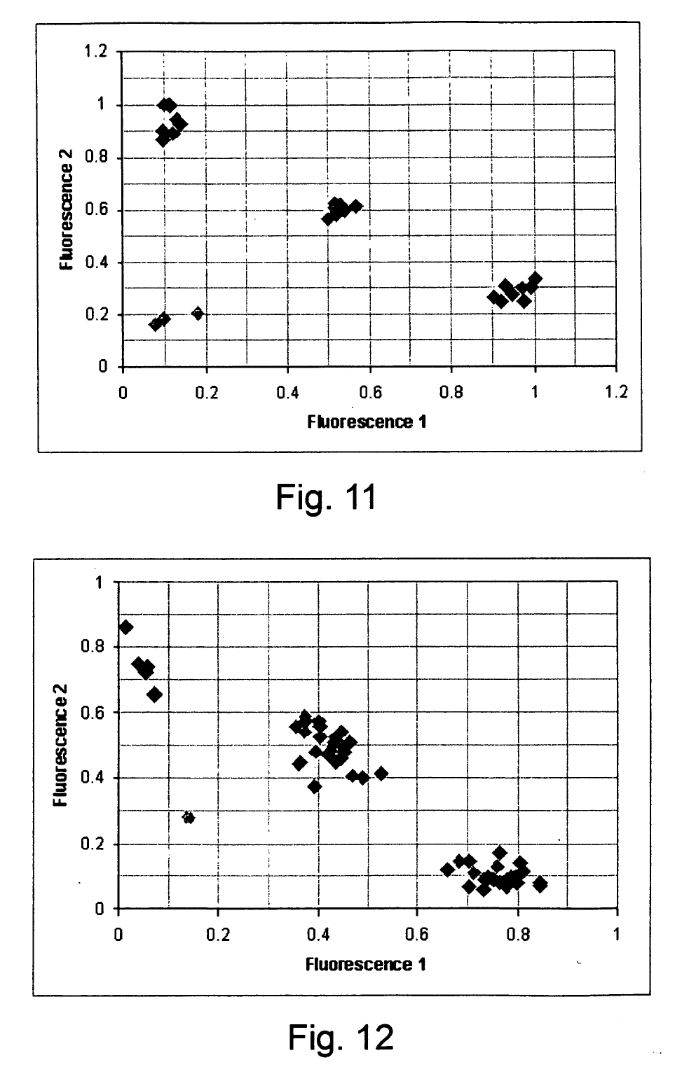

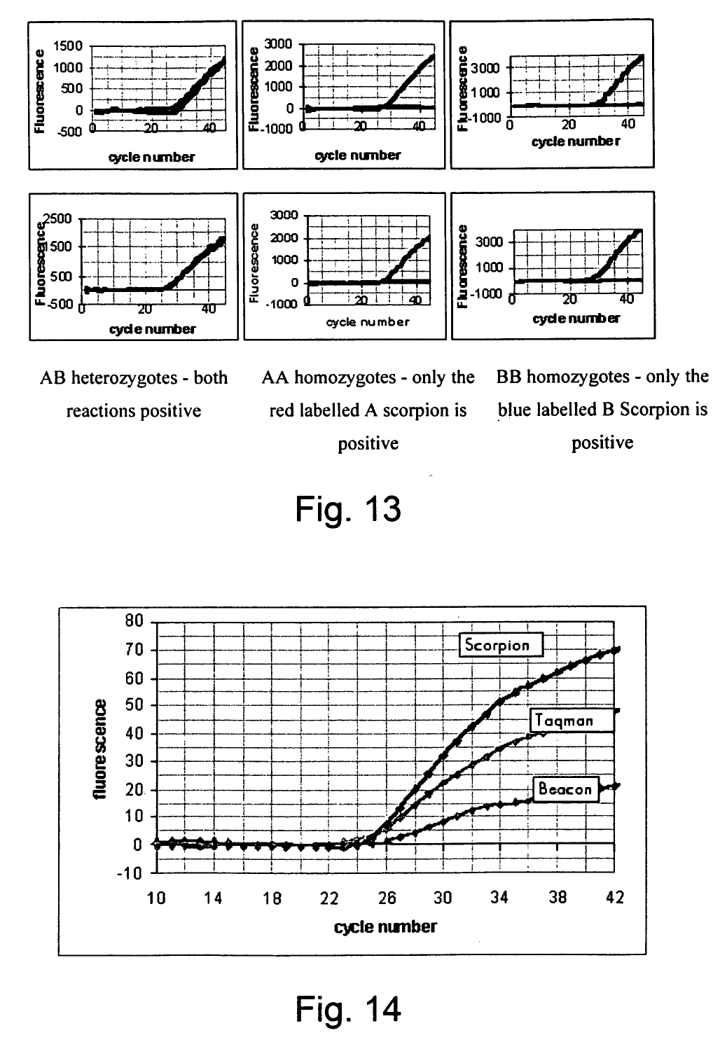

There are two ways to make a Scorpions reaction allele specific. The primer element of the Scorpion can be made to only amplify a specific allele or the probe element can be designed to only detect a specific allele. Genotyping can be carried out in real-time or more typically as a post-PCR end point analysis. Three variations are shown here:
1) End point genotyping of the MTHFR gene using allele specific primer Scorpions. The ratio of the two fluorescent dyes indicates the genotype (FIG. 11)
2) End point genotyping of the NAT2 gene using allele specific probe Scorpions (FIG. 12).
3) Real time single tube genotyping of the a BRCA1 polymorphism using allele specific primerS (FIG. 13).
   7) Comparison to other Technologies

The data shown in FIGS. 14-16 show three separate comparisons of Scorpions to both Molecular Beacons and Taqman detection systems. In all three cases, the probe element was identical—the Molecular Beacon contained an additional stem and the Scorpion coupled the probe to the primer.

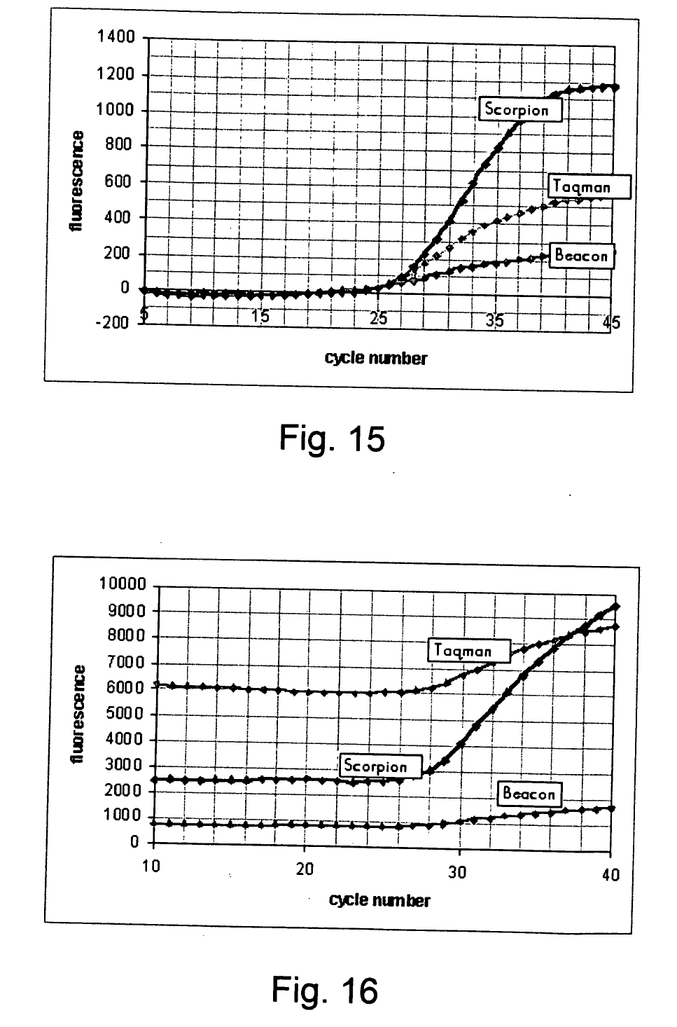

FIG. 14 shows Example I relating to the MTHFR gene. FIG. 15 shows Example II relating to the CYP gene. FIG. 16 shows Example III relating to the BRCA1 gene.

In Example III, the data has not been background corrected and shows the higher background associated with Taqman technology.

In all cases the Scorpions gave the strongest signal. There are many other examples where the scale of the difference between the Scorpions and the alternative detection systems is even bigger than shown here. Indeed there are several amplicons which cannot be detected by either Taqman or Beacons but work well with a Scorpions reaction. This is because the uni-molecular nature of the Scorpions reaction means that the desired interaction of the probe and the target occurs very rapidly and in advance of any competing side reactions such as the formation of internal secondary structures or reannealing. This also shows the broad applicability of the technology to different therapeutic areas, from smoking (CYP) to breast cancer (BRCA1).

Product Development

A diagnostic product development programme is available to provide both consultancy and practical support to companies. Projects of mutually agreed specification are undertaken from proof of concept to a CE marked diagnostic test.

The service draws on our experience of the technology to accelerate partner companies product launch to ensure early access to new markets.

Steps Involved in Product Development:
Design and plan
Prototype testing
Assay optimisation
Assay validation
   Technical
   Clinical
CE marking
   Product folder generation and review
   Launch
      Scorpions Licensing
Scorpions technology is available for license from DxS. Existing licensees for diagnostic applications include Ortho Clinical Diagnostics, Minerva Biolabs GmbH and Sangtec Molecular Diagnostics.

The present invention, in particular the Scorpion Technology and ARMS technology, can be used in conjunction with different platforms, such as cDNA chips, microarrays, and nanotechnological applications, as will be apparent to the skilled person.

### ARMS Technology

DxS have recently added Amplification Refractory Mutation System (ARMS™) to their portfolio of technologies that are available for licence. ARMS is a well-established primary method for the detection of SNP's and other genetic variation. ARMS is a simple, reliable and widely used method for the detection of gene mutations and single nucleotide polymorphisms (SNPs).

| Feature | Benefit | Applications | |
|---|---|---|---|
| Genotyping | Very reliable mutation | 1. | Genetic Testing |
| | detection method | 2. | Pharmacogenetics |
| | allowing detection of all | 3. | Personalised Medicine |
| | SNPs and indels | 4. | Predisposition Testing |
| | Converts non-specific | 5. | Research Genotyping |
| | PCR detection methods | | |
| | such as intercalation, | | |
| | Amplifluor and Lux into | | |
| | genotyping methods | | |
| Q-genotyping | Allows quantitative | 6. | Sample Pooling |
| | genotyping when | 7. | Viral Genotyping |
| | combined with real-time | 8. | Tumour Analysis |
| | PCR | 9. | Cancer Screening and |
| | Excellent SNP | | Early Detection |
| | discrimination ability | 10. | Cancer Monitoring |
| | allows detection of | | |
| | genetic variations when | | |
| | only a small proportion of | | |
| | the sample carries the | | |
| | mutation | | |
| Haplotyping | Combination of two | 11. | Haplotyping |
| | ARMS primers allows | 12. | HLA testing |
| | direct analysis of the | 13. | Promoter Analysis |
| | phase of associated SNPs | | |
| | Identifies whether SNPs | | |
| | are on the same | | |
| | chromosome | | |
| Multiplexing | Several ARMS primers | 14. | Multiplex Genetic |
| | can be combined into one | | Testing |
| | reaction | 15. | High throughput |
| | Compatible with micro- | | Genotyping |
| | array detection | | |

Therefore, the Nicotest method is for determining a personalised therapeutic regime for aiding a user quit smoking. The method comprises receiving genetic information relating to a patient by means of genetic analysis in a lab 70. This is then used by the diagnostic processor to determine genetic criteria relevant to the personalised therapeutic regime. Personal information is provided by the patient in the form of the questionnaire results. This is then used by the diagnostic processor to determine personal criteria relevant to the personalised therapeutic regime. Both the genetic criteria and the personal criteria and then combined using a decision matrix to determine the personalised therapeutic regime for the patient.

In a practical embodiment, a delivery platform for the method according to the invention combines a number of elements which include:
1. A quantitative module or genotyping test . . . this could also be a proteomic, metabolomic or similar genome related analysis that enables patient stratificationby genetic background/history.
2. A qualitative module or biomedical questionnaire specific to each individual . . . this could be supplied in a lifestyle context i.e. completed by a layman (but would be open to subjective input) as expected within the lead product or a professional context i.e. completed by a GP or Consultant clinician. It could also be an automated link with the Electronic Patient Record—especially if the output were to be focused on treatment recommendations for a prescribable drug.
3. A decision matrix—this combines the qualitative and quantitative to generate a Personalised Treatment Programme (PTP)/recommendation.
4. An interactive patient/professional mechanism that monitors progress and feedback and enables the original PTP to be adjusted if necessary to optimize treatment over the course of the full regime. This interactive element is maintained throughout the duration of the treatment regime.

Items 1 to 4 could all be all online. Online data processing enables mass data analysis and rapid data exchange between professional & patient. It offers the quickest form of results feedback and due to the bioinformatic nature it facilitates the creation and generation of significant volumes of data which can be analysed quicker for genetic and biomedical trends/associations.

The integration of IT within a standard logistics process reduces leadtime, turnaround time and optimises productivity levels in respect of eliminating data input/human error and hence will offer a higher service quality level as standard.

This type of infrastructure allows a range of sub-contractors to be included within the overall structure and yet the customer only perceive one contact point. This is important where credibility and trust is uppermost in their minds in respect of data confidentiality. They do not need to know that a number of different ‘suppliers’ together enable this product/service. It also allows us, as a company, to grow into international markets quicker than the usual Bricks and Mortar supply chain would be able to.

A recent study by researchers at the Transdisciplinary Tobacco Use Research Center (TTURC) of the University of Pennsylvania School of Medicine indicates that a smoker's genetic make-up may affect whether they quit or not while using either bupropion (Zyban) or nicotine replacement therapies (NRTs) such as the nicotine patch or nasal spray. The results appear in the August 2005 issue of Neuropsychopharmacology.

Although in the specific embodiment described above the Internet is used, this is not essential to the present invention. The present invention can be applied to an application shared between machines that communicate with each other, for example, over a network. Therefore, although the specific embodiment network uses the Internet, the present invention is applicable to any network whether it be a conventional landline network or a wireless network. More specifically, the present invention is applicable to the Internet, an intranet, an extranet, a local area network, a wide area network or a network employing wireless application protocol.

Many further variations and modifications will suggest themselves to those versed in the art upon making reference to the foregoing illustrative embodiments, which are given by way of example only, and which are not intended to limit the scope of the invention, that being determined by the appended claims.

1. A method of determining a personalised therapeutic regime, comprising:
receiving at least one of the following: genomic, proteomic, biochemical or metabolomic information relating to a patient;
determining at least one of genomic, proteomic, biochemical or metabolomic criteria relevant to a personalised therapeutic regime for the patient using the or each genomic, proteomic, biochemical or metabolomic information;
receiving personal information relating to the patient;
determining personal criteria relevant to the personalised therapeutic regime using the personal information;
combining the or each genomic, proteomic, biochemical or metabolomic criteria and the personal criteria to determine the personalised therapeutic regime for the patient.

2. The method of claim 1, wherein the determining of the personalised therapeutic regime for the patient comprises applying weightings to the or each genomic, proteomic, biochemical or metabolomic criteria and the personal criteria.

3. The method of claim 1, wherein the personal information comprises information relating to the patient or information about the patient's lifestyle.

4. The method of claim 1, wherein the personal information comprises any one or combination of the following:
the ethnicity of the patient;
the sex of the patient;
the weight of the patient;
the Body Mass Index of the patient:
age of the patient;
the incidence of a condition of potential interest for the personalised therapeutic regime in the patient's family; and
the environmental conditions of the patient.

5. The method of claim 1, wherein the personal information is obtained in the form of a questionnaire.

6. The method of claim 5, wherein the questionnaire is provided to the user over a communications network.

7. The method of claim 1, wherein the or each genomic, proteomic, biochemical or metabolomic information is obtained from analysis of a sample from the patient.

8. The method of claim 7, wherein the analysis comprises any of or a combination of the following:
genotyping;
haplotyping;
analysis of the patient's RNA;
analysis of the patient's proteome; and
analysis of the patient's metabolome.

9. The method of claim 1, wherein once the therapeutic regime for the patient has been determined, the personalised therapeutic regime is administered to the patient.

10. The method of claim 9, wherein once the personalised therapeutic regime has been administered to the patient, feedback information is received from the patient related to the effects of the personalised therapeutic regime.

11. The method of claim 10, further comprising using the feedback information to determine an updated personalised therapeutic regime according to the effects of the personalised therapeutic regime on the patient.

12. The method of claim 11, further comprising administering the updated personalised therapeutic regime to the patient.

13. The method of claim 9, wherein once the therapeutic regime has been administered, augmentation information is provided to the patient in order augment the personalised therapeutic regime.

14. The method of claim 13, wherein the augmentation information relates to any one of a combination of:
cognitive behavioural therapy;
managed care; and
professional medical advice.

15. The method of claim 13, wherein cognitive behavioural therapy is provided over a communications network.

16. The method of claim 1, further comprising receiving results information relating to the results of the personalised therapeutic regime, and using said results information when determining the personalised therapeutic regime for a second patient.

17. The method of claim 1, wherein the therapeutic regime comprises a regime to aid the patient cease smoking.

18. The method of claim 17, wherein said the or each genomic, proteomic, biochemical or metabolomic criteria relate to any one or more of the following:
the CYP2A6 gene;
the CYP2D6 gene;
the CYP2C19 gene:
any combination of alleles CC, CT and TT of the CYP2B6 gene;
any combination of alleles A1A1, A1A2 and A2A2 of the DRD2 gene;
any combination of alleles AA, AT and TATA of the TPH gene;
any of the three allele combinations of the dopamine transporter gene (DAT);
any combination of 444 G and/or A alleles, the 910 polymorphism, 1368 A and/or G alleles of the human dopamine beta-hydroxylase gene (DBH);
the 5-HTT serotonin transporter gene; and
the 1460 C allele of the human monoamine oxidase A (MAO A) gene.

19. The method of claim 17, wherein said the or each genomic, proteomic, biochemical or metabolomic criteria relate to any one or more of the following:
any of alleles CYP2A6*1 to CYP2A6*12 and the gene duplication CYP2A6* 1×2;
any of alleles CYP2B6*4, *6 and *16;
any of alleles TMPT*2, TMPT*3A and TMPT*3C;
any of alleles UGT1A1*28 (7/7), UGT1A1*28 (6/7) and UGT1A1*28 (6/6);
allele TAQ1A of the DRD2 gene;
allele ANKK1 “C” or ANKK1 “T” of the DRD2 gene;
allele DRD2-141 Ins C of the DRD2 gene; and
allele DRD2-141 Del C of the DRD2 gene.

20. The method of claim 17, wherein the personal information relates to one or more of the following:
the ethnicity of the patient;
smoking topography;
the body mass index of the patient;
the length of time the patient has been a smoker;
the average number of cigarettes smoked a day;
the type and/or strength of tobacco smoked; and
the type of filter used, if any, when smoking the tobacco.

21. The method of claim 1, wherein the personal therapeutic regime relates to a suitable regime for treating or ameliorating any one or combination of the following: obesity, autoimmune diseases such as hayfever and arthritis, allergies, heart disease, AIDS, conditions associated with anticoagulant deficiencies such as haemophilia, dyspepsia, conditions associated with elevated cholesterol levels; and cancer, including breast cancer, prostate cancer, bowel cancer, testicular cancer, cancers of the blood, and lung cancer.

22. The method of claim 1, wherein the personal therapeutic regime relates to a suitable regime for treating or ameliorating depression

23. The method of claim 22, wherein the depression is selected from the group consisting of: Major Depression, Dysthymic Disorder, Unspecified Depression, Adjustment Disorder (with Depression), Bipolar Depression, depressive disorders, anxiety disorders of panic disorder, with and without agoraphobia, social phobia/social anxiety disorder, obsessive compulsive disorder, and post traumatic stress disorder.

24. The method of claim 1, wherein the personal therapeutic regime relates to a suitable regime for treating or ameliorating smoking in combination with depression.

25. The method of claim 1, wherein the metabolomic information is the level of the metabolite Cotinine.

26. The method of claim 1, comprising the use of Cognitive Behavioural Therapy.

27. The method of claim 26, comprising the use of Computerised Cognitive Behavioural Therapy (CCBT).

28. Apparatus for determining a personalised therapeutic regime, comprising:
a memory adapted to store at least one of genomic, proteomic, biochemical or metabolomic information relating to a patient;
a memory adapted to store personal information relating to the patient;
a determination processor adapted to determine at least one of genomic, proteomic, biochemical or metabolomic criteria relevant to a personalised therapeutic regime for the patient using the or each genomic, proteomic, biochemical or metabolomic information, to determine personal criteria relevant to the personalised therapeutic regime using the personal information, and to combine the or each genomic, proteomic, biochemical or metabolomic criteria and the personal criteria to determine the personalised therapeutic regime for the patient.

29. A system for determining a personalised therapeutic regime, comprising:
a terminal adapted to receive a user indication of personal information relating to patient;
a data store adapted to store said personal information relating to the patient;
a testing sub-system adapted to perform at least one of genomic, proteomic, biochemical or metabolomic testing on a sample from the patient to obtain or each genomic, proteomic, biochemical or metabolomic information relating to the patient;
a data store adapted to store the or each genomic, proteomic, biochemical or metabolomic information;
a determination sub-system adapted to determine at least one of genomic, proteomic, biochemical or metabolomic criteria relevant to a personalised therapeutic regime for the patient using the or each genomic, proteomic, biochemical or metabolomic information, to determine personal criteria relevant to the personalised therapeutic regime using the personal information, and to combine the or each genomic, proteomic, biochemical or metabolomic criteria and the personal criteria to determine the personalised therapeutic regime for the patient.

30. A server for use in a system for determining a personalised therapeutic regime, comprising:
a data store adapted to store personal information relating to a patient;
a data store adapted to store at lest one of genomic, proteomic, biochemical or metabolomic information relating to the patient; and
a determination processor adapted to determine at least one of genomic, proteomic, biochemical or metabolomic criteria relevant to a personalised therapeutic regime for the patient using the or each genomic, proteomic, biochemical or metabolomic information, to determine personal criteria relevant to the personalised therapeutic regime using the personal information, and to combine the or each genomic, proteomic, biochemical or metabolomic criteria and the personal criteria to determine the personalised therapeutic regime for the patient.

31. A computer readable medium carrying computer readable code for controlling a system or server to carry out the method of claim 1.

32. The method of claim 1, wherein the determination of the personalized therapeutic treatment regimen for the patient comprises any one of a combination of the following:
a rule-based system; and
a probability based system.

33. The method of claim 32, wherein the rule-based system and/or probability-based system is updated based on new medical opinion.

34. The method of claim 1, wherein the determination of the personalized therapeutic treatment regimen for the patient comprises the use of a decision matrix.

35. Apparatus for determining a personalised therapeutic regime, comprising:
memory means for storing at least one of genomic, proteomic, biochemical or metabolomic information relating to a patient;
memory means for storing personal information relating to the patient;
determination means for determining at least one of genomic, proteomic, biochemical or metabolomic criteria relevant to a personalised therapeutic regime for the patient using the or each genomic, proteomic, biochemical or metabolomic information, to determine personal criteria relevant to the personalised therapeutic regime using the personal information, and to combine the or each genomic, proteomic, biochemical or metabolomic criteria and the personal criteria to determine the personalised therapeutic regime for the patient.

36. A system for determining a personalised therapeutic regime, comprising:
terminal means for receiving a user indication of personal information relating to patient;
memory means for storing said personal information relating to the patient;
testing sub-system means for performing at least one of genomic, proteomic, biochemical or metabolomic testing on a sample from the patient to obtain or each genomic, proteomic, biochemical or metabolomic information relating to the patient;
memory means for storing the or each genomic, proteomic, biochemical or metabolomic information;
determination sub-system means for determining at least one of genomic, proteomic, biochemical or metabolomic criteria relevant to a personalised therapeutic regime for the patient using the or each genomic, proteomic, biochemical or metabolomic information, for determining personal criteria relevant to the personalised therapeutic regime using the personal information, and for combining the or each genomic, proteomic, biochemical or metabolomic criteria and the personal criteria to determine the personalised therapeutic regime for the patient.

37. A server for use in a system for determining a personalised therapeutic regime, comprising:
memory means for storing personal information relating to a patient;
memory means for storing at least one of genomic, proteomic, biochemical or metabolomic information relating to the patient; and
determination means for determining at least one of genomic, proteomic, biochemical or metabolomic criteria relevant to a personalised therapeutic regime for the patient using the or each genomic, proteomic, biochemical or metabolomic information, for determining personal criteria relevant to the personalised therapeutic regime using the personal information, and for combining the or each genomic, proteomic, biochemical or metabolomic criteria and the personal criteria to determine the personalised therapeutic regime for the patient.

38. The method of claim 1, wherein the comparing comprises comparing feature data obtained from the patient with corresponding reference feature data for the specified drug.

39. A method of identifying a dosage of a specified drug, comprising:
receiving at least one of the following: genomic, proteomic, biochemical or metabolomic information relating to a patient;
determining at least one of genomic, proteomic, biochemical or metabolomic criteria; and
comparing the or each at least one of genomic, proteomic, biochemical or metabolomic criteria with reference criteria relating to an optimal dosage of said specified drug based on patients with said genomic, proteomic, biochemical or metabolomic criteria, to thereby determine the dosage of a specified drug.

40. A method of determining a personalised therapeutic regime, comprising:
determining at least one of genomic, proteomic, biochemical or metabolomic criteria, relating to a patient;
determining personal criteria for the patient relating to personal information of the patient;
applying scoring criteria to the or each genomic, proteomic, biochemical or metabolomic criteria and the personal criteria to determine a scoring result for the patient;
determining the personalised therapeutic regime for the patient by comparing the scoring result against predetermined reference scoring information.

41. The method of claim 40, further comprising:
receiving at least one of the following: genomic, proteomic, biochemical or metabolomic information relating to a patient; and
receiving personal information relating to the patient; wherein:
the or each genomic, proteomic, biochemical or metabolomic criteria is determined using the or each genomic, proteomic, biochemical or metabolomic information; and
the personal criteria is determined using the personal information.

42. The method of claim 41, wherein the personal information comprises any one or combination of the following:
the ethnicity of the patient;
the sex of the patient;
the incidence of a condition of potential interest for the personalised therapeutic regime in the patient's family;
information relating to the patient or information about the patient's lifestyle; and
the environmental conditions of the patient.

43. The method of claim 41, wherein the or each genomic, proteomic, biochemical or metabolomic information is obtained from analysis of a sample from the patient, and wherein the analysis comprises any of or a combination of the following:
genotyping;
haplotyping;
analysis of the patient's RNA;
analysis of the patient's proteome; and
analysis of the patient's metabolome.

44. The method of claim 40, wherein determining the scoring result for the patient comprises applying weightings to the or each genomic, proteomic, biochemical or metabolomic criteria and the personal criteria.

45. The method of claim 40, wherein:
determining the scoring result comprises comparing the or each at least one of genomic, proteomic, biochemical or metabolomic criteria with reaction reference criteria linking a specified drug to adverse reactions to said specified drug based on patients with said genomic, proteomic, biochemical or metabolomic criteria, to thereby determine whether a patient should be prescribed said specified drug or a pharmaceutical product comprising said specified drug.

46. The method of claim 40, wherein:
determining the scoring result comprises comparing the or each at least one of genomic, proteomic, biochemical or metabolomic criteria with dosage reference criteria relating to an optimal dosage of a drug in the personalised therapeutic regime for patients with said genomic, proteomic, biochemical or metabolomic criteria.
